(19) United States
(12) Patent Application Publication
(10) Pub. No.: US 2025/0339412 A1
(43) Pub. Date: Nov. 6, 2025

(54) METHODS OF TREATING SOLID TUMOR USING (19R)-5-CHLORO-3-ETHYL-16-FLUORO-10,19-DIMETHYL-20-OXA-3,4,10,11,23-PENTAAZAPENTACYCLO[19.3.1.02,6.08,12.013,18]PENTACOSA-1(24),2(6),4,8,11,13,15,17,21(25),22-DECAEN-22-AMINE

(71) Applicant: Nuvalent, Inc., Cambridge, MA (US)

(72) Inventors: Jennifer Anne GREEN, Miami Beach, FL (US); Jason T. KROPP, Westford, MA (US); Darlene NOCI, Burlington, MA (US); Henry Efrem PELISH, Newton, MA (US); James R. PORTER, Rowley, MA (US); John R. SOGLIA, Sherborn, MA (US); Anupong TANGPEERACHAIKUL, Cambridge, MA (US); Christopher Durant TURNER, Walpole, MA (US); Viola Weijia ZHU, Newton, MA (US); David James PEARSON, Derby (GB)

(21) Appl. No.: 18/854,274

(22) PCT Filed: Apr. 6, 2023
(86) PCT No.: PCT/US2023/065449

§ 371 Date: Oct. 4, 2024

Related U.S. Application Data
(60) Provisional application No. 63/328,620, filed on Apr. 7, 2022.
(60) Provisional application No. 63/328,609, filed on Apr. 7, 2022.
(60) Provisional application No. 63/356,702, filed on Jun. 29, 2022.
(60) Provisional application No. 63/376,962, filed on Sep. 23, 2022.

(51) Int. Cl. A61K 31/439; A61K 9/20; A61K 31/4545; A61K 31/506; A61K 31/5377; A61K 31/675; A61K 45/06; A61P 35/00; A61P 35/04

(52) U.S. Cl. CPC A61K 31/439; A61K 9/2009; A61K 9/2013; A61K 9/2054; A61K 31/4545; A61K 31/506; A61K 31/5377; A61K 31/675; A61K 45/06; A61P 35/00; A61P 35/04

(57) ABSTRACT

Provided herein are methods of using a heteroaromatic macrocyclic ether compound (e.g., Compound 1), or a stereoisomer, or a mixture of stereoisomers thereof, or a pharmaceutically acceptable salt thereof, for treating, preventing or managing solid tumor.

This application claims the benefit of priority to U.S. Ser. No. 63/328,609, filed Apr. 7, 2022, U.S. Ser. No. 63/328,620, filed Apr. 7, 2022, U.S. Ser. No. 63/356,702, filed Jun. 29, 2022, and U.S. Ser. No. 63/376,962, filed Sep. 23, 2022, each of which is incorporated herein by reference in its entirety.

### BACKGROUND

Receptor tyrosine kinases (RTKs) are cell surface enzymes that receive outside signals, such as whether to grow and divide, and transmit those signals in the cell through kinase activity. Many RTKs are proto-oncogenes; aberrant RTK activity can drive cell survival, growth and proliferation leading to cancer and related disorders. This aberrant kinase activity can be caused by mutations such as activating mutations in the kinase domain, gene rearrangements that result in fusion proteins containing the intact kinase domain, amplification and other means. RTK proto-oncogenes include ROS1, anaplastic lymphoma kinase (ALK), NTRK1 (encodes TRKA), NTRK2 (encodes TRKB), and NTRK3 (encodes TRKC).

ALK is an RTK proto-oncogene, with ALK rearrangements detected in many cancers, including NSCLC, anaplastic large cell lymphoma (ALCL), IMT, diffuse large B-cell lymphoma (DLBCL), esophageal squamous cell carcinoma (ESCC), renal medullary carcinoma, renal cell carcinoma, breast cancer, colon cancer, serous ovarian carcinoma, papillary thyroid cancer, cholangiocarcinoma and spitzoid tumors, and ALK activating mutations have been detected in neuroblastoma and anaplastic thyroid cancer. Oncogenic ALK gene fusions contain the kinase domain of ALK (3′ region) fused to the 5′ region of more than 20 different partner genes, the most common being EML4 in NSCLC and NPM in ALCL. Other partner genes include TMP1, WDCP, GTF2IRD1, TPM3, TPM4, CLTC, LMNA, PRKAR1A, RANBP2, TFG, FN1, KLC1, VCL, STRN, HIP1, DCTN1, SQSTM1, TPR, CRIM1, PTPN3, FBXO36, ATIC and KIF5B.kinases.

NTRK1, NTRK2 and NTRK3 are RTK proto-oncogenes that encode TRK-family kinases, with NTRK1, NTRK2 and NTRK3 chromosomal rearrangements detected at low frequency in many cancers. For treatment of ROS1-positive or ALK-positive patients, however, TRK inhibition, particularly in the central nervous system (CNS), has been associated with adverse reactions, including dizziness/ataxia/gait disturbance, paraesthesia, weight gain and cognitive changes.

Existing agents used to treat oncogenic ROS1 and ALK have substantial deficiencies. These deficiencies may represent one or more of the following: associated TRK inhibition, limited CNS activity, and inadequate activity against resistance mutations. Treatment of ROS1-positive or ALK-positive patients accompanied by TRK inhibition is associated with adverse reactions, particularly in the CNS, including dizziness/ataxia/gait disturbance, paraesthesia, weight gain and cognitive changes. Additionally, there is a need for CNS-penetrant and TRK-sparing inhibitors of the wild type ROS1 kinase domain and ROS1 with acquired resistance mutations occurring either individually or in combination, including G2032R, D2033N, S1986F, S1986Y, L2026M, L1951R, E1935G, L1947R, G1971E, E1974K, L1982F, F2004C, F2004V, E2020K, C2060G, F2075V, V2089M, V2098I, G2101A, D2113N, D2113G, L2155S, L2032K, and L2086F. Likewise, there is a need for CNS-penetrant and TRK-sparing inhibitors of ALK with acquired resistance mutations. A variety of ALK drug resistance mutations, occurring either individually or in combination, have been reported, including G1202R, F1174C, F1174L, I1171N, I1171S, I1171T, L1196M, V1180L, C1156Y, G1202del, G1202K, G1269A, F1174L, F1174S, S1206Y, E1210K, T1151M, T1151_L1152insT, D1203N, S1206Y, S1206C, L1152R, L1196Q, L1198P, L1198F, R1275Q, L1152P, C1156T, and F1245V, or a combination thereof.

### SUMMARY

Provided herein are methods of using a heteroaromatic macrocyclic ether compound (e.g., Compound 1), or a stereoisomer, or a mixture of stereoisomers thereof, or a pharmaceutically acceptable salt thereof, for treating, preventing or managing solid tumor.

In one embodiment, provided herein is a method of treating solid tumor, comprising administering to a subject in need thereof a therapeutically effective amount of Compound 1:

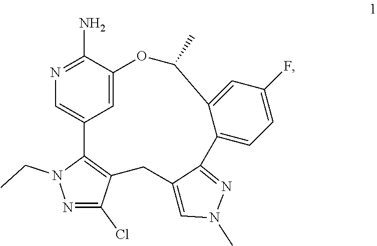 [SMILES: CCn1nc(Cl)c2c1-c1cnc(N)c(c1)O[C@H](C)c1cc(F)ccc1-c1nn(C)cc1C2]

or a stereoisomer, or a mixture of stereoisomers thereof, or a pharmaceutically acceptable salt thereof. In certain embodiments, the compound is Compound 1.

In one embodiment, the solid tumor is advanced ALK-positive solid tumor. In one embodiment, the solid tumor is locally advanced or metastatic solid tumor. In one embodiment, the solid tumor is advanced ALK-positive non-small cell lung cancer (NSCLC).

In one embodiment, the solid tumor is metastatic ALK-positive solid tumor. In one embodiment, the solid tumor is central nervous system (CNS) (e.g., brain) metastatic ALK-positive solid tumor. In one embodiment, the solid tumor is metastatic ALK-positive NSCLC. In one embodiment, the solid tumor is CNS (e.g., brain) metastatic ALK-positive NSCLC.

In one embodiment, the subject is naïve to tyrosine kinase inhibitor (TKI) therapy. In one embodiment, the subject has been treated with one or more prior TKI therapies. In one embodiment, the subject has been treated with one prior ALK TKI therapy (e.g., crizotinib, ceritinib, alectinib, brigatinib, or lorlatinib). In one embodiment, the subject has been treated with at least one prior ALK TKI therapies. In one embodiment, the subject has been treated with at least two prior ALK TKI therapies. In one embodiment, the subject has been treated with at least one prior ALK TKI therapy selected from the group consisting of ceritinib, alectinib, brigatinib, and lorlatinib. In one embodiment, the subject having the ALK fusion-positive NSCLC has been treated with at least one prior ALK TKI therapy, one of which is a second or third generation TKI selected from the group consisting of ceritinib, alectinib, brigatinib, and lorlatinib. In one embodiment, the subject having an ALK-positive solid tumor (e.g., NSCLC) has been treated with at least one prior systemic anticancer therapy. In one embodiment, the subject having an ALK-positive solid tumor (e.g., NSCLC) where no satisfactory standard therapy exists. In one embodiment, the subject having advanced ALK-positive NSCLC has been treated with one prior first generation ALK TKI therapy (e.g., crizotinib). In one embodiment, the subject having advanced ALK-positive NSCLC has been treated with one prior second generation ALK TKI therapy selected from ceritinib, alectinib, and brigatinib. In one embodiment, the subject having advanced ALK-positive NSCLC has been treated with two or three prior first or second generation ALK TKI therapies selected from crizotinib, ceritinib, alectinib, and brigatinib. In one embodiment, the subject having advanced ALK-positive NSCLC has been treated with two or three prior ALK TKI therapies with lorlatinib in the second or third line of therapy. In one embodiment, the subject having a solid tumor harboring an ALK rearrangement or activating ALK mutation has been treated with at least one prior systemic anticancer therapy. In one embodiment, the subject having a solid tumor harboring an ALK rearrangement or activating ALK mutation has no existing satisfactory standard therapy. In certain embodiments, the subject has an ALK resistance mutation (e.g., single mutation and/or compound mutation). In certain embodiments, the subject has an ALK G1202R. In one embodiment, the solid tumor is locally advanced or metastatic solid tumor. In certain embodiments, the advanced or metastatic ALK-positive NSCLC is locally advanced or metastatic ALK-positive NSCLC. In certain embodiments, the advanced or metastatic ALK-positive NSCLC is locally advanced or metastatic ALK-positive NSCLC harboring an ALK rearrangement.

In one embodiment, the compound is administered at an amount of from about 5 mg to about 400 mg once (QD) or twice (BID) daily. In one embodiment, the compound is administered at an amount of from about 15 mg to about 200 mg (by weight of Compound 1) once (QD) or twice (BID) daily. In one embodiment, the compound is administered at an amount of from about 15 mg to about 200 mg (by weight of Compound 1) once (QD) daily. In one embodiment, the compound is administered at an amount of from about 15 mg, 25 mg, 50 mg, 100 mg, 150 mg, or 200 mg (by weight of Compound 1) once (QD) daily.

In one embodiment, the compound is administered at an amount of from about 10 mg to about 150 mg (by weight of Compound 1) once (QD) or twice (BID) daily. In one embodiment, the compound is administered at an amount of from about 10 mg to about 150 mg (by weight of Compound 1) once (QD) daily. In one embodiment, the compound is administered at an amount of from about 10 mg, 15 mg, 25 mg, 50 mg, 100 mg, or 150 mg (by weight of Compound 1) once (QD) daily.

In one embodiment, the compound is administered at an amount of from about 10 mg to about 100 mg (by weight of Compound 1) once (QD) or twice (BID) daily. In one embodiment, the compound is administered at an amount of from about 10 mg to about 100 mg (by weight of Compound 1) once (QD) daily. In one embodiment, the compound is administered at an amount of from about 10 mg, 15 mg, 25 mg, 50 mg, or 100 mg (by weight of Compound 1) once (QD) daily.

In one embodiment, the compound is administered at an amount of from about 25 mg to about 250 mg (by weight of Compound 1) once (QD) or twice (BID) daily. In one embodiment, the compound is administered at an amount of from about 25 mg to about 250 mg (by weight of Compound 1) once (QD) daily. In one embodiment, the compound is administered at an amount of from about 25 mg, 50 mg, 100 mg, 150 mg, 200 mg, or 250 mg (by weight of Compound 1) once (QD) daily.

In one embodiment, the compound is administered to a subject with an empty stomach (e.g., at least 1 hour before and no sooner than 2 hours after ingestion of food and/or beverages other than water). In one embodiment, the compound is administered to a subject following ingestion of food and/or beverages. In one embodiment, the compound is administered in the absence of a strong inhibitor of CYP3A4 or a sensitive substrate of CYP3A4. In one embodiment, the compound is administered in the presence of a strong inhibitor of CYP3A4 or a sensitive substrate of CYP3A4. In one embodiment, the compound is administered in the absence of a sensitive substrate of CYP2C8. In one embodiment, the compound is administered in the presence of a sensitive substrate of CYP2C8. In one embodiment, the compound is administered in the absence of a substrate of P-gp/multidrug resistance protein 1 (MDR1), a substrate of BCRP/breast cancer resistance protein (ABCG2), OATP1B1, OATP1B3 or MATE1. In one embodiment, the compound is administered in the presence of a substrate of P-gp/multidrug resistance protein 1 (MDR1), a substrate of BCRP/breast cancer resistance protein (ABCG2), OATP1B1, OATP1B3 or MATE1. In one embodiment, the compound is administered in the absence of a gastric acid reducing agent (e.g. proton pump inhibitors). In one embodiment, the compound is administered in the presence of a gastric acid reducing agent. In one embodiment, the compound is administered in the absence of a strong inducer of CYP3A4. In one embodiment, the compound is administered in the presence of a strong inducer of CYP3A4.

### BRIEF DESCRIPTION OF DRAWINGS

FIG. 1 shows phase 1 and phase 2 study design.

FIG. 2 shows that Compound 1 induced regression in an MR619 cholangiocarcinoma (STRN-ALK G1202R) patient-derived xenograft model subcutaneously implanted in NSG mice. Vehicle was 20% HP-β-CD and was used to formulate Compound 1. Lorlatinib was tested at 5 mg/kg—a dose selected to approximate the free-drug exposure of the 100 mg QD human dose (Shaw et al. Lancet Oncol 2017; 18 (12): 1590; Yamazaki et al. J Pharm Exp Ther 2014; 351 (1): 67) and was formulated in 2 eq. HCl+20% HP-β-CD in water. All treatments are well-tolerated. Dosing was not performed on weekends; in other words, the dosing was performed 5 days on/2 days off. Average±SEM plotted. BID=twice per day; PO=administered orally.

FIG. 3 shows the activity of various inhibitors against ALK activating mutations in neuroblastoma. Biochemical assays were performed using purified ALK kinases and a fluorogenic substrate (PhosphoSens Assay, Shults and Imperiali. J Am Chem Soc 2003; 125(47):14248). Cell viability assays (3-day) were performed using Kelly (ALK F1174L), SH-SY5Y (ALK F1174L), and NB-1 (ALK Ex2-3del) human neuroblastoma cell lines. NB-1 is known to have ALK-overexpression but was identified to also harbor ALK partial deletion around exons 2-3 (Okubo et al. Oncogene 2012; 31(44):4667). All data from n≥2 repeat testing. Average plotted.

FIG. 4 shows the activity of various ALK inhibitors against the human anaplastic large-cell lymphoma cell line Karpas299 bearing NPM1-ALK fusion in a 3-day cell viability assay or ALK phosphorylation ELISA assay. Numerical IC₅₀ values are indicated above the bars. All data from n≥2 repeat testing. Average plotted.

FIG. 5 shows the activity of various ALK inhibitors against Aska-SS bearing ALK deletion around exons 2-17 in a 3-day cell viability assay and ALK phosphorylation ELISA assay. Numerical IC₅₀ values are indicated above the bars. All data from n≥2 repeat testing. Average plotted.

FIG. 6 shows the inhibition of cell viability by various inhibitors in the MR448re cell line bearing EML4-ALK v3 G1202R/T1151M mutation. IC₅₀ values shown are in nM.

FIG. 7 shows the inhibition of cell viability by various inhibitors in diverse cells bearing EML4-ALK fusion (with or without resistance mutations), ETV6-TRKB fusion, or TPM3-TRKA fusion. IC₅₀ (nM) values are plotted as heatmap.

FIG. 8A to FIG. 8D show the inhibition of pALK in PDX tumors harboring HIP1-ALK. Abbreviations: β2m=β2-microglobulin; BID=twice daily; HP-β-CD=2-hydroxypropyl-β-cyclodextrin; PO=orally; QD=once daily; SEM=standard error of the mean. Mice bearing PDX tumors harboring HIP1-ALK were treated with a single dose (QD×1) or BID×5 days (BID×5) PO of vehicle (20% HP-β-CD), Compound 1, or lorlatinib followed by tumor collection at the indicated time points (n=2 or 3) and analysis by western blot. FIG. 8A shows quantification of pALK following QD×1 administration. FIG. 8B shows quantification of pALK following BID×5 administration. FIG. 8C shows quantification of ALK following QD×1 administration. FIG. 8D shows quantification of ALK following BID×5 administration. Anti-β2m is a loading control that does not recognize mouse β2m.

FIG. 9A to FIG. 9G show the inhibition of signaling through the MAPK and PI3K/AKT pathways and induction of apoptosis in PDX tumors harboring HIP1-ALK. Abbreviations: β2m=B2-microglobulin; BID=twice daily; HP-β-CD=2-hydroxypropyl-β-cyclodextrin; PDX=patient-derived xenograft; PO=orally; QD=once daily; SEM=standard error of the mean. Mice bearing PDX tumors harboring HIP1-ALK were treated with a single dose (QD×1) or BID×5 days (BID×5) PO of vehicle (20% HP-β-CD), Compound 1, or lorlatinib, followed by tumor collection at the indicated time points (n=2 or 3) and western blot. Anti-β2m is a loading control that does not recognize mouse β2m. pERK is a marker of MAPK signaling, pAKT and pS6 are markers of PI3K/AKT signaling, and cleaved PARP is a marker of apoptosis. FIG. 9A shows quantification of pERK/ERK following QD×1 administration. FIG. 9B shows quantification of pERK/ERK following BID×5 administration. FIG. 9C shows quantification of pAKT/AKT following QD×1 administration. FIG. 9D shows quantification of pAKT/AKT following BID administration. FIG. 9E shows quantification of pS6/S6 following QD×1 administration. FIG. 9F shows quantification of pS6/S6 following BID×5 administration. FIG. 9G shows quantification of cleaved PARP/β2m following QD×1 administration.

FIG. 10 shows Compound 1 demonstrated potent antitumor activity in patient derived cell line (PDC) xenograft mouse model (MR448re cells (EML4-ALK_G1202R/T1151M expressing PDC) subcutaneously implanted nude mice).

FIG. 11 shows Compound 1 can overcome ALK G1202R compound mutations (BaF3 model).

FIG. 12 shows an overview of the food effect study design.

FIG. 13 shows an overview of the drug-drug interaction study design.

FIG. 14 is a representative XRPD pattern of Form 2 of free base of Compound 1.

FIG. 15 is a representative DSC thermogram of Form 2 of free base of Compound 1.

FIG. 16 is a representative DVS isotherm of Form 2 of free base of Compound 1.

FIG. 17 is a representative depiction of the unit cell an axis of single-crystal X-ray diffraction studies of Form 2 of free base of Compound 1.

FIG. 18 illustrates the plate layout for the viability assay portion of combination study with osimertinib.

FIG. 19 illustrates layout of conditions for the in vitro signaling pathway study portion of combination study with osimertinib.

FIG. 20A illustrates patient treatment history of YU-1077 cell line. FIG. 20B illustrates Sanger sequencing confirmation. E6:A20 denotes EML4 exon 6 fusion with ALK exon 20. NCBI sequence IDs for EML4 and ALK are provided.

FIG. 21 provides dose-response curves showing the activity of various compounds against YU-1077 cell line in a 3-day viability assay. Mean±SEM plotted, n=5. IC₅₀ values were calculated using fixed Emax=50%.

FIG. 22 shows western blot demonstrating the effects of ALK TKIs on ALK kinase activity (pALK) and downstream signaling (pERK, pAKT, pS6) in the YU-1077 cell line. Cells were treated for 6 hours. p=phosphorylated form of each protein marker.

FIG. 23A shows representative MRI images demonstrating YU-1077 tumor growth in the brain over 2 weeks, which was fully suppressed by Compound 1. Visible tumor mass is indicated with arrows. FIG. 23B shows 3D modeling of brain MRI scans to determine tumor volume. Visible tumor mass is highlighted with arrows and patterned fills. Images from individual mice are superimposed. All treatments were administered orally and were well-tolerated. Vehicle was 20% HP-β-CD and was used to formulate Compound 1. Alectinib was formulated in 20% PEG-400 and 3% Tween-80 in water.

FIG. 24A shows that Compound 1 suppressed YU-1077 tumor growth in the brain. Tumor volume was determined via 3D modeling. Mean±SEM plotted. The number of mice for each cohort is as indicated, except for the final datapoint for the alectinib cohort (n=6 instead of n=7). FIG. 24B shows survival analysis.

### DETAILED DESCRIPTION

### Definitions

Unless defined otherwise, all technical and scientific terms used herein have the meaning commonly understood by a person skilled in the art of the present disclosure. The following references provide one of skill with a general definition of many of the terms used in this disclosure: Singleton et al., Dictionary of Microbiology and Molecular Biology (2^{nd} ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

In some embodiments, chemical structures are disclosed with a corresponding chemical name. In case of conflict, the chemical structure controls the meaning, rather than the name.

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

Unless specifically stated or obvious from context, as used herein, the term “or” is understood to be inclusive. Unless specifically stated or obvious from context otherwise, as used herein, the terms “a”, “an”, and “the” are understood to be singular or plural. For example, when a compound provided herein is administered to “a patient”, it includes administering the compound to an individual patient or a patient population.

As used herein and unless otherwise specified, “stereoisomers” refer to the various stereoisomeric forms of a compound that comprises one or more asymmetric centers or stereohindrance in the structure. In some embodiments, a stereoisomer is an enantiomer, a mixture of enantiomers, an atropisomer, or a tautomer thereof. For example, the compounds described herein can be in the form of an individual enantiomer, diastereomer or geometric isomer (e.g. an atropisomer), or can be in the form of a mixture of stereoisomers, including racemic mixtures and mixtures enriched in one or more stereoisomer. In some embodiments, compounds provided herein may be atropisomers. In certain embodiments, atropisomers are stereoisomers arising because of hindered rotation about a single bond, where energy differences due to steric strain or other contributors create a barrier to rotation that is high enough to allow for isolation of individual conformers. Stereoisomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H. Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

In certain embodiments, compounds provided herein may be racemic. In certain embodiments, compounds provided herein may be enriched in one enantiomer. For example, a compound provided herein may have greater than about 30% ee, about 40% ee, about 50% ee, about 60% ee, about 70% ee, about 80% ee, about 90% ee, or even about 95% or greater ee. In certain embodiments, compounds provided herein may have more than one stereocenter. In certain such embodiments, compounds provided herein may be enriched in one or more diastereomer. For example, a compound provided herein may have greater than about 30% de, about 40% de, about 50% de, about 60% de, about 70% de, about 80% de, about 90% de, or even about 95% or greater de.

In certain embodiments, the therapeutic preparation may be enriched to provide predominantly one enantiomer of a compound. An enantiomerically enriched mixture may comprise, for example, at least about 60 mol percent of one enantiomer, or more particularly at least about 75, about 90, about 95, or even about 99 mol percent. In certain embodiments, the compound enriched in one enantiomer is substantially free of the other enantiomer, wherein substantially free means that the substance in question makes up less than about 10%, or less than about 5%, or less than about 4%, or less than about 3%, or less than about 2%, or less than about 1% as compared to the amount of the other enantiomer, e.g., in the composition or compound mixture. For example, if a composition or compound mixture contains about 98 grams of a first enantiomer and about 2 grams of a second enantiomer, it would be said to contain about 98 mol percent of the first enantiomer and only about 2% of the second enantiomer.

In certain embodiments, the therapeutic preparation may be enriched to provide predominantly one diastereomer of a compound. A diastereomerically enriched mixture may comprise, for example, at least about 60 mol percent of one diastereomer, or more particularly at least about 75, about 90, about 95, or even about 99 mol percent.

In some embodiments, a moiety in a compound exists as a mixture of tautomers. A “tautomer” is a structural isomer of a moiety or a compound that readily interconverts with another structural isomer. For example, a pyrazole ring has two tautomers:

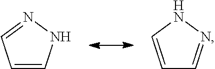 [SMILES: c1cn[nH]c1 ; c1cn[nH]c1]

which differ in the positions of the pi-bonds and a hydrogen atom. Unless explicitly stated otherwise, a drawing of one tautomer of a moiety or a compound encompasses all of the possible tautomers.

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. In certain embodiments, the subject is a human. In certain embodiments, the subject is a human adult at least of 40 years old. In certain embodiments, the subject is a human adult at least of 50 years old. In certain embodiments, the subject is a human adult at least of 60 years old. In certain embodiments, the subject is a human adult at least of 70 years old. In certain embodiments, the subject is a human adult at least of 18 years old or at least of 12 years old. As used herein and unless otherwise specified, a human subject to which administration of a therapeutic (e.g., a compound as described herein) is contemplated in order to treat, prevent or manage a disease, disorder, or condition, or symptoms thereof, is also called a “patient”.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample. These effects are also called “prophylactic” effects. Thus, as used herein and unless otherwise specified, the terms “prevention” and “preventing” refer to an approach for obtaining beneficial or desired results including, but not limited, to prophylactic benefit. For prophylactic benefit, a therapeutic can be administered to a patient at risk of developing a particular disease, or to a patient reporting one or more of the physiological symptoms of a disease, even though a diagnosis of this disease may not have been made. In one embodiment, a therapeutic is administered prior to clinical manifestation of the unwanted condition (e.g., disease or other unwanted state of the subject) for prophylactic benefit (e.g., it protects the subject against developing the unwanted condition).

As used herein and unless otherwise specified, the terms “treatment” and “treating” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment. In one embodiment, “treatment” comprises administration of a therapeutic after manifestation of the unwanted condition (i.e., it is intended to diminish, ameliorate, or stabilize the existing unwanted condition or side effects thereof).

As used herein and unless otherwise specified, “cancer” refers to any malignant and/or invasive growth or tumor caused by abnormal cell growth, including solid tumors named for the type of cells that form them, cancer of blood, bone marrow, or the lymphatic system. Examples of solid tumors include but not limited to sarcomas and carcinomas. Examples of cancers of the blood include but not limited to leukemias, lymphomas and myeloma. Cancer includes, but not limited to a primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of different type from latter one.

As used herein and unless otherwise specified, “abnormal cell growth” refers to cell growth that is independent of normal regulatory mechanisms (e.g., loss of contact inhibition). Abnormal cell growth may be benign (not cancerous), or malignant (cancerous). In some embodiments of the methods provided herein, the abnormal cell growth is cancer.

In some embodiments, the abnormal cell growth is cancer mediated by an anaplastic lymphoma kinase (ALK). In some such embodiments, the ALK is a genetically altered ALK. In other embodiments, the abnormal cell growth is cancer mediated by ROS1 kinase. In some such embodiments, the ROS1 kinase is a genetically altered ROS1 kinase. In some embodiments, the abnormal cell growth is cancer, in particular NSCLC. In some such embodiments, the NSCLC is mediated by ALK or ROS1. In specific embodiments, the cancer is NSCLC is mediated by genetically altered ALK or genetically altered ROS1.

As used herein and unless otherwise indicated, the term “managing” encompasses preventing the recurrence of the particular disease or disorder in a patient who had suffered from it, lengthening the time a patient who had suffered from the disease or disorder remains in remission, reducing mortality rates of the patients, and/or maintaining a reduction in severity or avoidance of a symptom associated with the disease or condition being managed.

An “effective amount”, as used herein, refers to an amount that is sufficient to achieve a desired biological effect. A “therapeutically effective amount”, as used herein, refers to an amount that is sufficient to achieve a desired therapeutic effect. For example, a therapeutically effective amount can refer to an amount that is sufficient to improve at least one sign or symptom of cancer.

A “response” to a method of treatment can include a decrease in or amelioration of negative symptoms, a decrease in the progression of a disease or symptoms thereof, an increase in beneficial symptoms or clinical outcomes, a lessening of side effects, stabilization of disease, partial or complete remedy of disease, among others.

As used herein and unless otherwise indicated, the term “relapsed” refers to a disorder, disease, or condition that responded to prior treatment (e.g., achieved a complete response) then had progression. The prior treatment can include one or more lines of therapy.

As used herein and unless otherwise indicated, the term “refractory” refers to a disorder, disease, or condition that has not responded to prior treatment that can include one or more lines of therapy.

As used herein, and unless otherwise specified, the terms “about” and “approximately,” when used in connection with doses, amounts, or weight percents of ingredients of a composition or a dosage form, mean a dose, amount, or weight percent that is recognized by one of ordinary skill in the art to provide a pharmacological effect equivalent to that obtained from the specified dose, amount, or weight percent. In certain embodiments, the terms “about” and “approximately,” when used in this context, contemplate a dose, amount, or weight percent within 30%, within 20%, within 15%, within 10%, or within 5%, of the specified dose, amount, or weight percent.

As used herein and unless otherwise specified, the terms “about” and “approximately,” when used in connection with a numeric value or a range of values which is provided to characterize a particular solid form, e.g., a specific temperature or temperature range, such as, for example, that describing a melting, dehydration, desolvation or glass transition temperature; a mass change, such as, for example, a mass change as a function of temperature or humidity; a solvent or water content, in terms of, for example, mass or a percentage; or a peak position, such as, for example, in analysis by IR or Raman spectroscopy or XRPD; indicate that the value or range of values may deviate to an extent deemed reasonable to one of ordinary skill in the art while still describing the particular solid form. For example, in particular embodiments, the terms “about” and “approximately,” when used in this context, indicate that the numeric value or range of values may vary within 25%, 20%, 15%, 10%, 9%, 8%, 7%, 6%, 5%, 4%, 3%, 2%, 1.5%, 1%, 0.5%, or 0.25% of the recited value or range of values. For example, in some embodiments, the value of XRPD peak position may vary by up to ±0.2 degrees 2θ while still describing the particular XRPD peak. In one embodiment, the value of XRPD peak position may vary by up to ±0.1 degrees 2θ. In one embodiment, the value of XRPD peak position may vary by up to ±0.05 degrees 2θ.

The term “between” includes the endpoint numbers on both limits of the range. For example, the range described by “between 3 and 5” is inclusive of the numbers “3” and “5”.

As used herein and unless otherwise specified, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in. J. Pharmaceutical Sciences (1977) 66:1-19. In certain embodiments, pharmaceutically acceptable salts include, but are not limited to, alkyl, dialkyl, trialkyl or tetra-alkyl ammonium salts. In certain embodiments, pharmaceutically acceptable salts include, but are not limited to, L-arginine, benenthamine, benzathine, betaine, calcium hydroxide, choline, decanol, diethanolamine, diethylamine, 2-(diethylamino) ethanol, ethanolamine, ethylenediamine, N-methylglucamine, hydrabamine, 1H-imidazole, lithium, L-lysine, magnesium, 4-(2-hydroxyethyl) morpholine, piperazine, potassium, 1-(2-hydroxyethyl) pyrrolidine, sodium, triethanolamine, tromethamine, and zinc salts. In certain embodiments, pharmaceutically acceptable salts include, but are not limited to, Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Pharmaceutically acceptable anionic salts include, but are not limited to, acetate, aspartate, benzenesulfonate, benzoate, besylate, bicarbonate, bitartrate, bromide, camsylate, carbonate, chloride, citrate, decanoate, edetate, esylate, fumarate, gluceptate, gluconate, glutamate, glycolate, hexanoate, hydroxynaphthoate, iodide, isethionate, lactate, lactobionate, malate, maleate, mandelate, mesylate, methylsulfate, mucate, napsylate, nitrate, octanoate, oleate, pamoate, pantothenate, phosphate, polygalacturonate, propionate, salicylate, stearate, acetate, succinate, sulfate, tartrate, teoclate, and tosylate.

As used herein and unless otherwise specified, the term “solid form” and related terms refer to a physical form which is not predominantly in a liquid or a gaseous state. Solid forms may be crystalline, amorphous or mixtures thereof.

### Compounds

In one embodiment, the compound used in the methods provided herein is a compound of the following formula, also referred to as “Compound 1” or a compound of Formula (I):

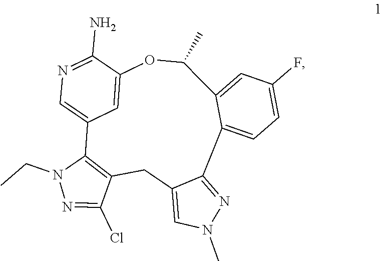 [SMILES: CCn1nc(Cl)c2c1-c1cnc(N)c(c1)O[C@H](C)c1cc(F)ccc1-c1nn(C)cc1C2]

or a stereoisomer, or a mixture of stereoisomers thereof, or a pharmaceutically acceptable salt thereof. Compound 1 has the chemical name of (19R)-5-chloro-3-ethyl-16-fluoro-10,19-dimethyl-20-oxa-3,4,10,11,23-pentaazapentacyclo[19.3.1.0^{2,6}.0^{8,12}.0^{13,18}]pentacosa-1(24),2(6),4,8,11,13,15,17,21(25),22-decaen-22-amine, and is described in International Application Publication No. WO 2021/226269, the entirety of which is incorporated herein by reference. As used herein, “Compound 1,” “Compound 1 free base,” “Compound 1 (free base),” and “free base Compound 1” are used interchangeably.

It is to be understood that, unless otherwise specified, when the compound as described herein is provided as a pharmaceutically acceptable salt thereof, that the weight amount refers to the portion exclusive of the salt portion (i.e. as the Compound 1 free base).

In one embodiment, Compound 1 (free base) is used in the methods provided herein. In one embodiment, a tautomer of Compound 1 is used in the methods provided herein. In one embodiment, Compound 1 substantially free of the(S)-enantiomer is used in the methods provided herein. As used herein, “substantially free” means the(S)-enantiomer is present in less than about 10 wt % of Compound 1 (e.g. less than about 5 wt %, less than about 1 wt %, less than about 0.05 wt %, less than about 0.02 wt %, or less than about 0.01 wt % of Compound 1). In one embodiment, Compound 1 is substantially pure with the enantiomeric purity of at least about 98% (e.g. about 99%, 99.5%, 99.8%, or 99.9%).

In one embodiment, the(S)-enantiomer of Compound 1 is used in the methods provided herein. In one embodiment, a tautomer of the(S)-enantiomer of Compound 1 is used in the methods provided herein. In one embodiment, a pharmaceutically acceptable salt of the(S)-enantiomer of Compound 1 is used in the methods provided herein.

In one embodiment, the racemic mixture of Compound 1 is used in the methods provided herein. In one embodiment, a tautomer of the racemic mixture of Compound 1 is used in the methods provided herein. In one embodiment, a pharmaceutically acceptable salt of the racemic mixture of Compound 1 is used in the methods provided herein.

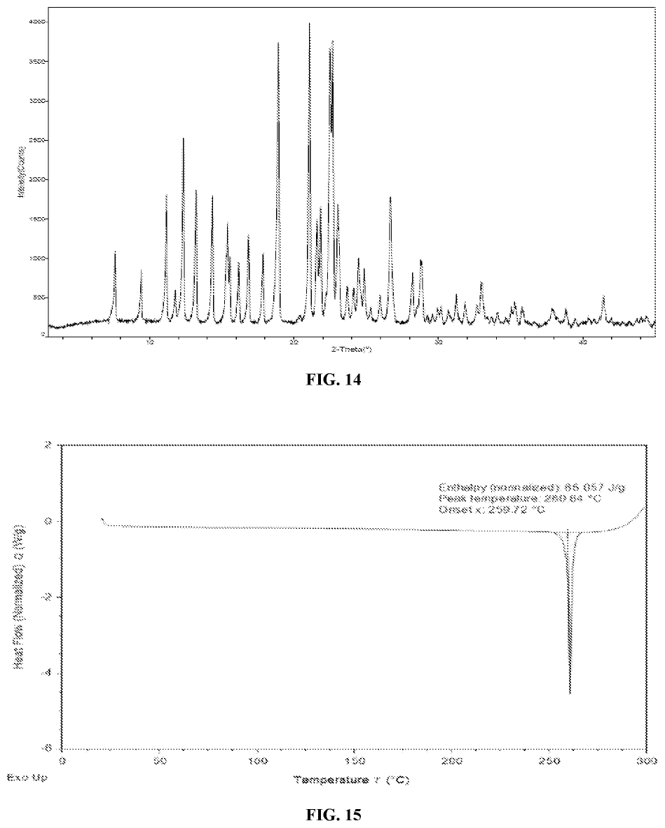

In one embodiment, a solid form of Compound 1 is used in the methods provided herein. In one embodiment, a solid form of a free base of Compound 1 is used in the methods provided herein. In one embodiment, a Form 2 of Compound 1 is used in the methods provided herein. A representative XRPD pattern of Form 2 of Compound 1 is provided in FIG. 14.

In one embodiment, the solid form comprising a free base of Compound 1 used in the methods provided herein is characterized by 1, 2, 3, 4, 5, 6, 7, 8, 9, 10, 11, 12, 13, 14, 15, 16, 17, 18, 19, 20, 21 or all of the XRPD peaks located at approximately the following positions (e.g., degrees 2θ±0.2) when measured using Cu Kα radiation: 7.6, 9.4, 11.2, 12.4, 13.2, 14.3, 15.4, 15.6, 16.2, 16.9, 17.9, 18.9, 21.1, 21.6, 21.8, 22.5, 22.7, 23.0, 24.5, 24.9, 27.0, and 28.8° 2θ. In one embodiment, the solid form is characterized by at least 3 of the peaks. In one embodiment, the solid form is characterized by at least 5 of the peaks. In one embodiment, the solid form is characterized by at least 7 of the peaks. In one embodiment, the solid form is characterized by at least 9 of the peaks. In one embodiment, the solid form is characterized by at least 11 of the peaks. In one embodiment, the solid form is characterized by all of the peaks.

In one embodiment, the solid form comprising a free base of Compound 1 used in the methods provided herein is characterized by an XRPD pattern, when measured using Cu Kα radiation, comprising at least three peaks selected from the group consisting of approximately (e.g., ±0.2°) 11.2, 12.4, 13.2, 14.3, 18.9, 21.1, 21.6, 21.8, 22.5, 22.7, 23.0, and 27.0° 2θ. In one embodiment, the solid form is characterized by an XRPD pattern comprising at least four peaks selected from the group consisting of approximately (e.g., ±0.2°) 11.2, 12.4, 13.2, 14.3, 18.9, 21.1, 21.6, 21.8, 22.5, 22.7, 23.0, and 27.0° 2θ. In one embodiment, the solid form is characterized by an XRPD pattern comprising at least five peaks selected from the group consisting of approximately (e.g., ±0.2°) 11.2, 12.4, 13.2, 14.3, 18.9, 21.1, 21.6, 21.8, 22.5, 22.7, 23.0, and 27.0° 2θ.

In one embodiment, the solid form comprising a free base of Compound 1 used in the methods provided herein is characterized by an XRPD pattern comprising peaks at approximately (e.g., ±0.2°) 12.4, 18.9, and 21.1° 2θ. In one embodiment, the XRPD pattern further comprises a peak at approximately (e.g., ±0.2°) 13.2 and 22.5° 2θ. In one embodiment, the XRPD pattern further comprises peaks at approximately (e.g., ±0.2°) 11.2 and 22.7° 2θ. In one embodiment, the XRPD pattern comprises peaks at approximately (e.g., ±0.2°) 11.2, 12.4, 13.2, 14.3, 18.9, 21.1, 21.8, 22.5, 22.7, 23.0, and 27.0° 2θ.

In one embodiment, the solid form is characterized by an XRPD pattern that matches the XRPD pattern depicted in FIG. 14.

In one embodiment, an XRPD pattern described herein is obtained using Cu Kα radiation. In one embodiment, the XRPD pattern is measured by XRPD using Cu Kα radiation comprising Kai radiation having a wavelength of 1.5406 Å and Kaz radiation having a wavelength of 1.5444 Å, wherein the Kα₁:Kα₂ ratio is 0.5.

A representative DSC thermogram of Form 2 is provided in FIG. 15. In one embodiment, the solid form comprising a free base of Compound 1 used in the methods provided herein exhibits, as characterized by DSC, a thermal event (endothermic) with an onset temperature of about 260° C. (e.g. ±2°). In one embodiment, the thermal event has a peak temperature of about 261° C. (e.g. ±2°). In one embodiment, without being bound by a particular theory, the thermal event corresponds to melting. In one embodiment, the solid form is characterized by a DSC thermogram that matches the DSC thermogram depicted in FIG. 15. In one embodiment, the DSC thermogram is as measured by DSC using a scanning rate of about 10° C./minute.

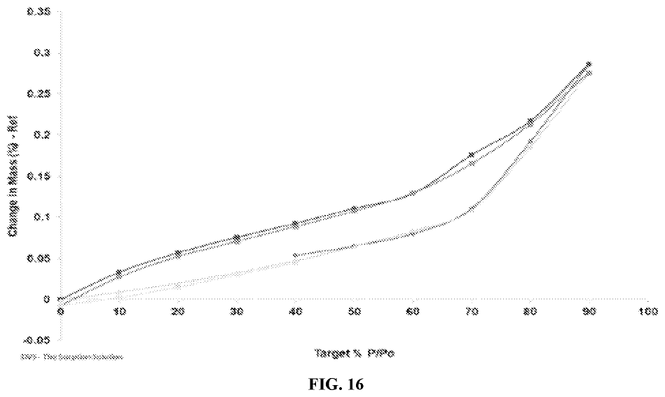

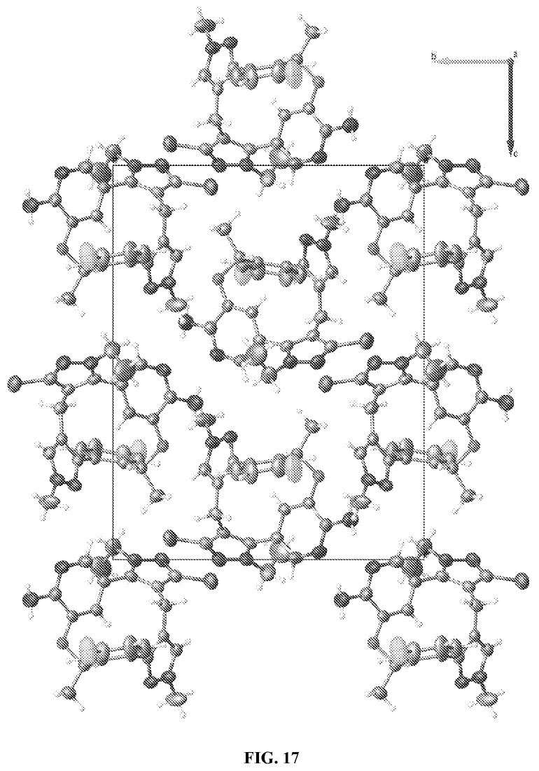

A representative DVS isotherm of Form 2 is provided in FIG. 16. In one embodiment, the solid form comprising a free base of Compound 1 used in the methods provided herein exhibits a weight increase of about 0.3% (e.g. +0.05%) when subjected to an increase in relative humidity from about 0 to about 90% relative humidity. In one embodiment, the solid form is characterized by a DVS isotherm that matches the DVS isotherm depicted in FIG. 16. In one embodiment, the DVS isotherm is as measured at about 25° C.

In one embodiment, the solid form comprising a free base of Compound 1 used in the methods provided herein is characterized by approximate unit cell dimensions of: a=8.2 Å, b=14.8 Å, c=18.7 Å, α=90°, β=90°, and γ=90°. In one embodiment, Form 2 has approximately unit cell dimensions of: a=8.17 Å, b=14.75 Å, c=18.69 Å, α=90°, β=90°, and γ=90°. In one embodiment, Form 2 has approximately unit cell dimensions of: a=8.169 Å, b=14.750 Å, c=18.694 Å, α=90°, β=90°, and γ=90°. In one embodiment, Form 2 has a unit cell of a space group of P2₁2₁2₁. In one embodiment, Form 2 has a volume of about 2252.4 Å³/cell. In one embodiment, Form 2 has a Z value of 4. In one embodiment, Form 2 has a density of about 1.336 g/cm³.

In one embodiment, the solid form comprising a free base of Compound 1 used in the methods provided herein is anhydrous. In one embodiment, the solid form is a crystalline anhydrous free base of Compound 1. In one embodiment, the solid form is substantially free of amorphous Compound 1. In one embodiment, the solid form is substantially free of other crystalline forms of Compound 1. In one embodiment, the solid form is substantially free of salts of Compound 1. In one embodiment, the solid form is not solvated. In one embodiment, one or more residual solvent may be present in the solid form, but the residual solvent does not form a solvate of Compound 1. In one embodiment, the solid form is substantially pure. In one embodiment, the solid form is substantially chemically pure. In one embodiment, the solid form is about over 95 wt % chemically pure. In one embodiment, the solid form is about over 96 wt % chemically pure. In one embodiment, the solid form is about over 97 wt % chemically pure. In one embodiment, the solid form is about over 98 wt % chemically pure. In one embodiment, the solid form is about over 99 wt % chemically pure. In one embodiment, the solid form is substantially enantiomerically pure. In one embodiment, the solid form is about at least 98% enantiomerically pure. In one embodiment, the solid form is about at least 99% enantiomerically pure. In one embodiment, the solid form is about at least 99.5% enantiomerically pure. In one embodiment, the solid form is substantially physically pure.

In one embodiment, the solid form comprising a free base of Compound 1 used in the methods provided herein is a solid form comprising Form 2 of a free base of Compound 1.

### Methods of Use

In one embodiment, provided herein are methods of treating cancer comprising administering a heteroaromatic macrocyclic ether compound, such as Compound 1, or a stereoisomer, or a mixture of stereoisomers thereof, or a pharmaceutically acceptable salt thereof.

In one embodiment, provided herein are methods of using a heteroaromatic macrocyclic ether compound (e.g., Compound 1), or a stereoisomer, or a mixture of stereoisomers thereof, or a pharmaceutically acceptable salt thereof, for treating, preventing or managing solid tumor.

In one embodiment, provided herein is a method of treating a subject with solid tumor, comprising administering to said subject a therapeutically effective amount of Compound 1:

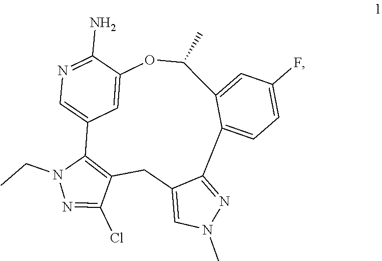 [SMILES: CCn1nc(Cl)c2c1-c1cnc(N)c(c1)O[C@H](C)c1cc(F)ccc1-c1nn(C)cc1C2]

or a stereoisomer, or a mixture of stereoisomers thereof, or a pharmaceutically acceptable salt thereof.

In one embodiment, the cancer is lung cancer, bile duct cancer, colorectal cancer, angiosarcoma, sarcoma, hemangioendothelioma, esophageal cancer, kidney cancer, breast cancer, colon cancer, thyroid cancer, neuroblastoma, hematological cancer, anaplastic large cell lymphoma (ALCL), atypical meningioma, breast cancer, cholangiocarcinoma, gastric cancer, glioblastoma, inflammatory myofibroblastic tumor (IMT), inflammatory hepatocellular adenoma (HCA), melanoma, pancreatic cancer, papillary thyroid carcinoma, salivary gland carcinoma, serous ovarian carcinoma, or spitzoid neoplasm.

In one embodiment, the solid tumor is advanced solid tumor. In one embodiment, the solid tumor is locally advanced or metastatic solid tumor. In one embodiment, the advanced solid tumor is relapsed after, refractory to, or resistant to the prior treatment by a tyrosine kinase inhibitor (TKI). In one embodiment, the solid tumor is non-small cell lung cancer (NSCLC). In one embodiment, the solid tumor is advanced NSCLC. In one embodiment, the solid tumor is metastatic. In one embodiment, the solid tumor is CNS metastatic. In one embodiment, the solid tumor is metastatic NSCLC. In one embodiment, the solid tumor is CNS metastatic NSCLC. As used herein and unless otherwise specified, “advanced tumor” refers to a tumor that cannot be cured or grows beyond the initial site of origin, either locally advanced or metastatic.

In one embodiment, the solid tumor (or cancer) is ALK positive. In one embodiment, the solid tumor is ALK positive NSCLC. In one embodiment, the solid tumor is advanced or metastatic ALK positive solid tumor. In one embodiment, the solid tumor is advanced ALK positive solid tumor. In one embodiment, the solid tumor is advanced ALK positive NSCLC. In one embodiment, the solid tumor is metastatic ALK positive solid tumor. In one embodiment, the solid tumor is locally advanced or metastatic ALK positive solid tumor. In one embodiment, the solid tumor is locally advanced ALK positive solid tumor. In one embodiment, the solid tumor is CNS metastatic ALK positive solid tumor. In one embodiment, the solid tumor is metastatic ALK positive NSCLC. In one embodiment, the solid tumor is CNS metastatic ALK positive NSCLC. In one embodiment, the solid tumor is a locally advanced or metastatic solid tumor harboring an ALK rearrangement or activating ALK mutation.

In one embodiment, the ALK positive solid tumor or cancer is anaplastic large cell lymphoma, inflammatory myofibroblastic tumors, diffuse large B-cell lymphoma, esophageal squamous cell carcinoma, renal medullary carcinoma, renal cell carcinoma, breast cancer, colorectal cancer, ovarian cancer, papillary thyroid carcinoma, cholangiocarcinoma, spitzoid tumors, neuroblastoma, or anaplastic thyroid cancer. In certain embodiments, the ALK positive solid tumor is anaplastic thyroid cancer. In one embodiment, the subject has not been treated with a prior therapy. In one embodiment, the subject is naïve to (i.e. not receiving) any tyrosine kinase inhibitor (TKI) therapy.

In one embodiment, the subject has been treated with one or more prior therapies. In one embodiment, the subject has been treated with at least one prior TKI therapy. In one embodiment, the subject has been treated with at least two prior TKI therapies. In one embodiment, the subject has been treated with one prior TKI therapy. In one embodiment, the subject has been treated with two prior TKI therapies. In one embodiment, the subject has been treated with three prior TKI therapies. In one embodiment, the TKI is ALK TKI. In one embodiment, the ALK TKI is crizotinib, ceritinib, alectinib, brigatinib, or lorlatinib. In certain embodiments, the subject with ALK positive NSCLC has previously received at least one ALK TKI. In certain embodiments, one of the prior ALK TKI is a 2^{nd} or 3^{rd} generation TKI (e.g. ceritinib, alectinib, brigatinib, or lorlatinib). In certain embodiments, the subject with the solid tumor has previously received at least one prior systemic anticancer therapy. In certain embodiments, the subject with the solid tumor has no satisfactory standard therapy. In certain embodiments, the subject with locally advanced or metastatic ALK positive NSCLC has received 1 prior 2^{nd}-generation ALK TKI (ceritinib, alectinib, brigatinib, or lorlatinib). In certain embodiments, the subject with locally advanced or metastatic ALK positive NSCLC has received 2-3 prior 1^{st} or 2nd-generation ALK TKIs (crizotinib, ceritinib, alectinib, brigatinib, or lorlatinib). In certain embodiments, the subject with locally advanced or metastatic ALK positive NSCLC has received 2-3 prior ALK TKIs, with lorlatinib in the 2^{nd} or 3^{rd} line.

In one embodiment, the subject has not been treated with prior systemic anticancer therapies. In one embodiment, the subject has been treated with up to one prior systemic anticancer therapies. In one embodiment, the subject has been treated with at least one prior systemic anticancer therapies. In one embodiment, the subject has been treated with at least two prior systemic anticancer therapies. In one embodiment, the subject has been treated with one prior systemic anticancer therapies. In one embodiment, the subject has been treated with two prior systemic anticancer therapies.

In one embodiment, the subject has not been treated with prior chemotherapy. In one embodiment, the subject has been treated with prior chemotherapy. In one embodiment, the subject has been treated with up to two prior chemotherapy. In one embodiment, the subject has been treated with at least one prior chemotherapy. In one embodiment, the subject has been treated with at least two prior chemotherapy. In one embodiment, the subject has been treated with one prior chemotherapy. In one embodiment, the subject has been treated with two prior chemotherapy.

In one embodiment, the subject has not been treated with prior immunotherapy. In one embodiment, the subject has been treated with prior immunotherapy. In one embodiment, the subject has been treated with up to two prior immunotherapy. In one embodiment, the subject has been treated with at least one prior immunotherapy. In one embodiment, the subject has been treated with at least two prior immunotherapy. In one embodiment, the subject has been treated with one prior immunotherapy. In one embodiment, the subject has been treated with two prior immunotherapy.

As used herein, “immunotherapy” refers to the treatment of a disease by activating or suppressing the immune system. Immunotherapies designed to elicit or amplify an immune response are classified as activation immunotherapies, while immunotherapies that reduce or suppress are classified as suppression immunotherapies. The immunotherapy can regulate the immune effector cells (e.g. lymphocytes, macrophages, dendritic cells, natural killer cells (NK Cell), cytotoxic T lymphocytes (CTL), etc.) to work together against cancer by targeting abnormal antigens expressed on the surface of tumor cells. Exemplified immunotherapy includes but not limited to checkpoint inhibitors (e.g. anti-cytotoxic T-lymphocyte-associated protein 4 (CTLA-4), anti-programmed cell death protein 1 (PD-1) inhibitors, and programmed death-ligand 1 (PD-L1) inhibitors). Exemplified PD-1 inhibitors include but are not limited to pembrolizumab (Keytruda), nivolumab (Opdivo), and cemiplimab (Libtayo). Exemplified PD-L1 inhibitors include but are not limited to atezolizumab (Tecentriq), avelumab (Bavencio), durvalumab (Imfinzi). Exemplified CTLA-4 inhibitor includes but is not limited to ipilimumab (Yervoy).

In one embodiment, the solid tumor is advanced or metastatic ALK positive NSCLC, and the subject has been treated with at least one prior ALK TKI therapy. In one embodiment, the at least one prior ALK TKI therapy is ceritinib, alectinib, or brigatinib. In one embodiment, the at least one prior ALK TKI therapy is crizotinib, ceritinib, alectinib, or brigatinib. In one embodiment, the at least one prior ALK TKI therapy is ceritinib, alectinib, brigatinib, or lorlatinib. In one embodiment, the at least one prior ALK TKI therapy is crizotinib, ceritinib, alectinib, brigatinib, or lorlatinib. In one embodiment, the subject has been treated with one or more prior systemic anticancer therapies. In one embodiment, the solid tumor is advanced or metastatic ALK positive NSCLC, and the subject has progressed on a prior therapy.

In one embodiment, the solid tumor is advanced or metastatic ALK positive NSCLC, and the subject has been treated with one prior ALK TKI therapy. In one embodiment, the prior ALK TKI therapy is ceritinib, alectinib, or brigatinib. In one embodiment, the at least one prior ALK TKI therapy is crizotinib, ceritinib, alectinib, or brigatinib. In one embodiment, the at least one prior ALK TKI therapy is ceritinib, alectinib, brigatinib, or lorlatinib. In one embodiment, the at least one prior ALK TKI therapy is crizotinib, ceritinib, alectinib, brigatinib, or lorlatinib. In one embodiment, the subject has been treated with ≤2 prior chemotherapy and/or immunotherapy. In one embodiment, the subject has been treated with more than two prior chemotherapy and/or more than two prior immunotherapy.

In one embodiment, the solid tumor is advanced or metastatic ALK positive NSCLC, and the subject has been treated with two or three prior ALK TKI therapies. In one embodiment, the two or three prior ALK TKI therapies are selected from crizotinib, ceritinib, alectinib, brigatinib, and lorlatinib. In one embodiment, the subject has been treated with ≤2 prior chemotherapy and/or immunotherapy. In one embodiment, the subject has been treated with more than two prior chemotherapy and/or more than two prior immunotherapy.

In one embodiment, the solid tumor is advanced or metastatic ALK positive NSCLC, and the subject has been treated with two or three prior ALK TKI therapies. In one embodiment, the solid tumor is advanced or metastatic ALK positive NSCLC, and the subject has been treated with one prior ALK TKI therapy and one ALK TKI is a 2^{nd} or 3^{rd} generation TKI (such as ceritinib, alectinib, brigatinib, or lorlatinib). In one embodiment, the second or third prior ALK TKI therapy is lorlatinib. In one embodiment, the subject has been treated with ≤2 prior chemotherapy and/or immunotherapy. In one embodiment, the subject has been treated with more than two prior chemotherapy and/or more than two prior immunotherapy.

In one embodiment, the subject has normal renal function. In one embodiment, the subject has a creatinine clearance of no less than about 90 mL/min (e.g. per Cockcroft-Gault formula). In one embodiment, the subject has a creatinine clearance of no less than about 80 mL/min (e.g. per Cockcroft-Gault formula). In one embodiment, the subject has a creatinine clearance of no less than about 60 ml/min (e.g. per Cockcroft-Gault formula).

In one embodiment, the compound used herein (Compound 1 or a stereoisomer, or a mixture of stereoisomers thereof, or a pharmaceutically acceptable salt thereof) is administered once daily (QD). In one embodiment, the compound is administered twice daily (BID). In certain embodiments, the compound used herein is Compound 1.

In one embodiment, the compound used herein (Compound 1 or a stereoisomer, or a mixture of stereoisomers thereof, or a pharmaceutically acceptable salt thereof) is administered at an amount of from about 5 mg to about 400 mg (by weight of the free base Compound 1) per day. In one embodiment, the compound is administered at an amount of from about 15 mg to about 200 mg per day. In one embodiment, the compound is administered at an amount of from about 20 mg to about 200 mg per day. In one embodiment, the compound is administered at an amount of from about 25 mg to about 200 mg per day. In one embodiment, the compound is administered at an amount of from about 50 mg to about 200 mg per day. In one embodiment, the compound is administered at an amount of from about 5 mg to about 150 mg per day. In one embodiment, the compound is administered at an amount of from about 15 mg to about 150 mg per day. In one embodiment, the compound is administered at an amount of from about 20 mg to about 150 mg per day. In one embodiment, the compound is administered at an amount of from about 25 mg to about 150 mg per day. In one embodiment, the compound is administered at an amount of from about 15 mg to about 125 mg per day. In one embodiment, the compound is administered at an amount of from about 20 mg to about 125 mg per day. In one embodiment, the compound is administered at an amount of from about 25 mg to about 125 mg per day. In one embodiment, the compound is administered at an amount of from about 15 mg to about 100 mg per day. In one embodiment, the compound is administered at an amount of from about 20 mg to about 100 mg per day. In one embodiment, the compound is administered at an amount of from about 25 mg to about 100 mg per day. In one embodiment, the compound is administered at an amount of from about 50 mg to about 250 mg per day. In one embodiment, the compound is administered at an amount of from about 50 mg to about 200 mg per day. In one embodiment, the compound is administered at an amount of from about 50 mg to about 150 mg per day. In one embodiment, the compound is administered at an amount of from about 50 mg to about 125 mg per day. In one embodiment, the compound is administered at an amount of from about 50 mg to about 100 mg per day. In one embodiment, the compound is administered at an amount of from about 10 mg to about 150 mg per day. In one embodiment, the compound is administered at an amount of from about 10 mg to about 100 mg per day. In one embodiment, the compound is administered at an amount of from about 10 mg to about 50 mg per day. In one embodiment, the compound is administered at an amount of from about 10 mg to about 25 mg per day. In one embodiment, the compound is administered at an amount of from about 15 mg to about 150 mg per day. In one embodiment, the compound is administered at an amount of from about 15 mg to about 100 mg per day. In one embodiment, the compound is administered at an amount of from about 15 mg to about 50 mg per day. In one embodiment, the compound is administered at an amount of from about 25 mg to about 150 mg per day. In one embodiment, the compound is administered at an amount of from about 50 mg to about 150 mg per day. In one embodiment, the compound is administered at an amount of from about 100 mg to about 150 mg per day.

In one embodiment, the compound is administered at an amount of about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200 mg, about 210 mg, about 220 mg, about 230 mg, about 240 mg, or about 250 mg per day.

In one embodiment, the amount is about 5 mg per day. In one embodiment, the amount is about 10 mg per day. In one embodiment, the amount is about 15 mg per day. In one embodiment, the amount is about 20 mg per day. In one embodiment, the amount is about 25 mg per day. In one embodiment, the amount is about 30 mg per day. In one embodiment, the amount is about 35 mg per day. In one embodiment, the amount is about 40 mg per day. In one embodiment, the amount is about 45 mg per day. In one embodiment, the amount is about 50 mg per day. In one embodiment, the amount is about 75 mg per day. In one embodiment, the amount is about 100 mg per day. In one embodiment, the amount is about 125 mg per day. In one embodiment, the amount is about 150 mg per day. In one embodiment, the amount is about 200 mg per day. In one embodiment, the amount is about 250 mg per day. As used herein, the weight amount refers to the weight amount of the free base Compound 1. In certain embodiments, the compound used herein is Compound 1.

In one embodiment, the compound used herein (Compound 1 or a stereoisomer, or a mixture of stereoisomers thereof, or a pharmaceutically acceptable salt thereof) is administered at an amount of from about 5 mg to about 400 mg (by weight of the free base Compound 1) once daily. In one embodiment, the compound is administered at an amount of from about 10 mg to about 250 mg once daily. In one embodiment, the compound is administered at an amount of about 15 mg to about 200 mg once daily. In one embodiment, the compound is administered at an amount of about 25 mg to about 200 mg once daily. In one embodiment, the compound is administered at an amount of from about 25 mg to about 200 mg once daily. In one embodiment, the compound is administered at an amount of from about 50 mg to about 200 mg once daily. In one embodiment, the compound is administered at an amount of from about 5 mg to about 150 mg once daily. In one embodiment, the compound is administered at an amount of from about 10 mg to about 100 mg once daily. In one embodiment, the compound is administered at an amount of about 10 mg to about 150 mg once daily. In one embodiment, the compound is administered at an amount of about 15 mg to about 150 mg once daily. In one embodiment, the compound is administered at an amount of about 25 mg to about 150 mg once daily. In one embodiment, the compound is administered at an amount of about 50 mg to about 150 mg once daily. In one embodiment, the compound is administered at an amount of about 100 mg to about 150 mg once daily. In one embodiment, the compound is administered at an amount of about 20 mg to about 150 mg once daily. In one embodiment, the compound is administered at an amount of from about 25 mg to about 150 mg once daily. In one embodiment, the compound is administered at an amount of about 25 mg to about 100 mg once daily. In one embodiment, the compound is administered at an amount of from about 25 mg to about 50 mg once daily. In one embodiment, the compound is administered at an amount of from about 25 mg to about 125 mg once daily. In one embodiment, the compound is administered at an amount of from about 10 mg to about 100 mg once daily. In one embodiment, the compound is administered at an amount of from about 15 mg to about 100 mg once daily. In one embodiment, the compound is administered at an amount of from about 15 mg to about 50 mg once daily. In one embodiment, the compound is administered at an amount of from about 25 mg to about 100 mg once daily. In one embodiment, the compound is administered at an amount of from about 25 mg to about 100 mg once daily. In one embodiment, the compound is administered at an amount of from about 50 mg to about 100 mg once daily. In one embodiment, the compound is administered at an amount of from about 50 mg to about 125 mg once daily. In one embodiment, the compound is administered at an amount of from about 50 mg to about 100 mg once daily. In one embodiment, the compound is administered at an amount of about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200 mg, about 210 mg, about 220 mg, about 230 mg, about 240 mg, or about 250 mg once daily. In one embodiment, the amount is about 5 mg once daily. In one embodiment, the amount is about 10 mg once daily. In one embodiment, the amount is about 15 mg once daily. In one embodiment, the amount is about 20 mg once daily. In one embodiment, the amount is about 25 mg once daily. In one embodiment, the amount is about 30 mg once daily. In one embodiment, the amount is about 35 mg once daily. In one embodiment, the amount is about 40 mg once daily. In one embodiment, the amount is about 45 mg once daily. In one embodiment, the amount is about 50 mg once daily. In one embodiment, the amount is about 75 mg once daily. In one embodiment, the amount is about 100 mg once daily. In one embodiment, the amount is about 125 mg once daily. In one embodiment, the amount is about 150 mg once daily. In one embodiment, the amount is about 200 mg once daily. In one embodiment, the amount is about 250 mg once daily. As used herein, the weight amount refers to the weight amount of the free base Compound 1. In certain embodiments, the compound used herein is Compound 1.

In one embodiment, the compound used herein (Compound 1 or a stereoisomer, or a mixture of stereoisomers thereof, or a pharmaceutically acceptable salt thereof) is administered at an amount of from about 5 mg to about 400 mg (by weight of the free base Compound 1) twice daily. In one embodiment, the compound is administered at an amount of from about 5 mg to about 250 mg twice daily. In one embodiment, the compound is administered at an amount of from about 15 mg to about 250 mg twice daily. In one embodiment, the compound is administered at an amount of from about 25 mg to about 250 mg twice daily. In one embodiment, the compound is administered at an amount of from about 15 mg to about 200 mg twice daily. In one embodiment, the compound is administered at an amount of from about 10 mg to about 150 mg twice daily. In one embodiment, the compound is administered at an amount of from about 10 mg to about 100 mg twice daily. In one embodiment, the compound is administered at an amount of from about 20 mg to about 200 mg twice daily. In one embodiment, the compound is administered at an amount of from about 25 mg to about 200 mg twice daily. In one embodiment, the compound is administered at an amount of from about 5 mg to about 100 mg twice daily. In one embodiment, the compound is administered at an amount of from about 15 mg to about 100 mg twice daily. In one embodiment, the compound is administered at an amount of from about 10 mg to about 50 mg twice daily. In one embodiment, the compound is administered at an amount of from about 15 mg to about 50 mg twice daily. In one embodiment, the compound is administered at an amount of from about 10 mg to about 100 mg twice daily. In one embodiment, the compound is administered at an amount of from about 25 mg to about 100 mg twice daily. In one embodiment, the compound is administered at an amount of from about 50 mg to about 100 mg twice daily. In one embodiment, the compound is administered at an amount of from about 5 mg to about 150 mg twice daily. In one embodiment, the compound is administered at an amount of from about 10 mg to about 150 mg twice daily. In one embodiment, the compound is administered at an amount of from about 15 mg to about 150 mg twice daily. In one embodiment, the compound is administered at an amount of from about 20 mg to about 150 mg twice daily. In one embodiment, the compound is administered at an amount of from about 25 mg to about 150 mg twice daily. In one embodiment, the compound is administered at an amount of about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200 mg, about 210 mg, about 220 mg, about 230 mg, about 240 mg, or about 250 mg twice daily. In one embodiment, the amount is about 5 mg twice daily. In one embodiment, the amount is about 10 mg twice daily. In one embodiment, the amount is about 15 mg twice daily. In one embodiment, the amount is about 20 mg twice daily. In one embodiment, the amount is about 25 mg twice daily. In one embodiment, the amount is about 30 mg twice daily. In one embodiment, the amount is about 35 mg twice daily. In one embodiment, the amount is about 40 mg twice daily. In one embodiment, the amount is about 45 mg twice daily. In one embodiment, the amount is about 50 mg twice daily. In one embodiment, the amount is about 75 mg twice daily. In one embodiment, the amount is about 100 mg twice daily. In one embodiment, the amount is about 125 mg twice daily. In one embodiment, the amount is about 150 mg twice daily. In one embodiment, the amount is about 200 mg twice daily. In one embodiment, the amount is about 250 mg twice daily. As used herein, the weight amount refers to the weight amount of the free base Compound 1. In certain embodiments, the compound used herein is Compound 1.

In one embodiment, the compound is administered orally.

In one embodiment, the compound is administered in the form of one or more tablets. In one embodiment, the tablet has a unit dose strength of about 5 mg by weight of the free base Compound 1. In one embodiment, the tablet has a unit dose strength of about 50 mg by weight of the free base Compound 1. In one embodiment, the tablet has a unit dose strength of about 75 mg by weight of the free base Compound 1. In one embodiment, the tablet has a unit dose strength of about 100 mg by weight of the free base Compound 1. In one embodiment, the tablet has a unit dose strength of about 125 mg by weight of the free base Compound 1. In one embodiment, the tablet has a unit dose strength of about 150 mg by weight of the free base Compound 1.

In one embodiment, the compound is administered to a subject with an empty stomach. In one embodiment, the compound is administered to a subject at least 1 hour before and no sooner than 2 hours after ingestion of food and/or beverages other than water. In one embodiment, the compound is administered to a subject with a full stomach. In one embodiment, the compound is administered with the ingestion of food and/or beverages.

In one embodiment, the subject experiences improvement in one or more symptoms selected from the group consisting of cognitive impairment, mood disorders, sleep disturbances, dizziness, ataxia, seizure, psychotic effect, speech disorder, and weight gain, after the administration of compound 1 (e.g. compared to the prior TKI treatment). In one embodiment, the subject does not experience one or more symptoms selected from the group consisting of cognitive impairment, mood disorders, sleep disturbances, dizziness, ataxia, seizure, psychotic effect, speech disorder, and weight gain, after the administration of the compound. In one embodiment, the subject experiences reduced levels of one or more of pALK, pERK, pAKT, and phospho-S6, after the administration of the compound. In one embodiment, the subject experiences reduced levels of pALK, after the administration of the compound. In one embodiment, the subject experiences reduced levels of pERK, after the administration of the compound. In one embodiment, the subject experiences reduced levels of pAKT, after the administration of the compound. In one embodiment, the subject experiences reduced levels of phospho-S6, after the administration of the compound. In one embodiment, the subject experiences increased levels of cleaved PARP, after the administration of the compound. In one embodiment, the subject experiences reduced activity of MAP kinase pathway, PI3K/AKT pathway, or JAK/STAT pathway, or any combination thereof in tumor, after the administration of the compound. In one embodiment, the subject experiences reduced activity of MAP kinase pathway, PI3K/AKT pathway, or JAK/STAT pathway, or any combination thereof in solid tumor, after the administration of the compound. In one embodiment, the subject experiences reduced activity of MAP kinase pathway in tumor, after the administration of the compound. In one embodiment, the subject experiences reduced activity of MAP kinase pathway in solid tumor, after the administration of the compound. In one embodiment, the subject experiences reduced activity of PI3K/AKT pathway in tumor, after the administration of the compound. In one embodiment, the subject experiences reduced activity of PI3K/AKT pathway in solid tumor, after the administration of the compound. In one embodiment, the subject experiences reduced activity of JAK/STAT pathway in tumor, after the administration of the compound. In one embodiment, the subject experiences reduced activity of JAK/STAT pathway in solid tumor, after the administration of the compound. In one embodiment, the subject experiences increased expression level of one or more marker of apoptosis in tumor, after the administration of the compound. In one embodiment, the subject experiences increased expression level of one or more marker of apoptosis in solid tumor, after the administration of the compound. In one embodiment, the subject experiences decreased level of one or more marker of proliferation (e.g. Ki 67) in tumor, after the administration of the compound. In one embodiment, the change in level of one or more marker provided herein is experienced 1 hour after the administration of the compound. In one embodiment, the change in level of one or more marker provided herein is experienced 12 hours after the administration of the compound.

In some embodiments, the cancer is a disease of uncontrolled cell proliferation that results from alterations in certain genes. Some of these alterations occur in genes that encode receptor tyrosine kinases (RTKs), a family of membrane-bound proteins that transmit signals from outside the cell to promote cell survival, growth, and proliferation. Aberrant RTK activation can lead to excessive cell growth and hence cancer. Generally, RTKs contain an N-terminal domain that binds extracellular ligands, a transmembrane domain, and a C-terminal kinase domain that catalyzes intracellular signal transduction.

In some embodiments, Compound 1 is an inhibitor of human anaplastic lymphoma kinase (ALK). ALK, also known as cluster of differentiation 246 (CD246), is an RTK encoded by the ALK gene. ALK and ROS1 are evolutionarily related; both belong to the insulin receptor superfamily, and their kinase domains share around 80% sequence similarity. A few ALK ligands in humans have been identified, including pleiotrophin and midkine growth factors. While the roles of ALK in humans remain inconclusive, much evidence from mouse studies suggests that it is important for the development of the nervous system. Like ROS1, ALK chromosomal rearrangements also lead to constitutively active fusion proteins that promote oncogenic transformation through MAPK, JAK/STAT, or other signaling pathways. ALK rearrangements represent 3-5% of NSCLC, roughly half of anaplastic large-cell lymphoma (ALCL), and a subset of many other cancers, with the predominant fusions being EML4-ALK for NSCLC and NPM1-ALK for ALCL. Oncogenic point mutations and amplification of ALK have also been observed, albeit at a much lower frequency than translocations. Crizotinib, ceritinib, alectinib, brigatinib, and lorlatinib are FDA-approved TKIs for the treatment of ALK-positive NSCLC and other cancers, either in front-line or after prior therapy. Crizotinib, for example, shows an overall response rate of 60-80% and median progression-free survival of 8-11 months, which is comparable to its activity in ROS1-positive NSCLC. Despite an initial response, many resistance mutations have emerged to the aforementioned FDA-approved TKIs. Some of these mutations, such as the combined L1196M gatekeeper and G1202R solvent front mutation, are resistant to all of the approved drugs. New treatments of ALK-positive cancer harboring resistance mutations are a need in the art.

In one embodiment, a compound provided herein selectively inhibits ALK. In one embodiment, the compound selectively inhibits ALK over ROS1. By way of non-limiting example, the ratio of selectivity can be greater than a factor of about 1.5, greater than a factor of about 2, than a factor of about 3, greater than a factor of about 4, greater than a factor of about 5, or greater than a factor of about 10, where selectivity can be measured by ratio of IC50 values, among other means. In one embodiment, the selectivity of ALK over ROS1 is measured by the ratio of the IC50 value against ROS1 to the IC50 value against ALK.

In one embodiment, the compound selectively inhibits ALK over TRK (e.g., TRKA, TRKB, and/or TRBC). By way of non-limiting example, the ratio of selectivity can be greater than a factor of about 5, greater than a factor of about 10, greater than a factor of about 50, greater than a factor of about 100, greater than a factor of about 200, greater than a factor of about 400, greater than a factor of about 600, greater than a factor of about 800, greater than a factor of about 1000, greater than a factor of about 1500, greater than a factor of about 2000, greater than a factor of about 5000, or greater than a factor of about 10,000, where selectivity can be measured by ratio of IC50 values, among other means. In one embodiment, the selectivity of ALK over TRK is measured by the ratio of the IC50 value against TRK to the IC50 value against ALK.

In one embodiment, provided herein is a method for selectively inhibiting ALK over ROS1 wherein the inhibition takes place in a cell. In one embodiment, provided herein is a method for selectively inhibiting ALK over TRK (e.g., TRKA, TRKB, and/or TRBC) wherein the inhibition takes place in a cell. In one embodiment, the method comprises contacting ALK with an effective amount of a compound provided herein. In an embodiment, such contact occurs in a cell. In an embodiment, such contact occurs in a cell in a mammal such as a human. In an embodiment, such contact occurs in a cell in human subject having a cancer provided herein.

In one embodiment, provided herein is a method for selectively inhibiting ALK over ROS1 wherein the inhibition takes place in a subject suffering from cancer, said method comprising administering an effective amount of a compound or a pharmaceutical composition provided herein to said subject. In certain embodiments, provided herein is a method of treating a subject suffering from a cancer associated with ALK, said method comprising selectively inhibiting ALK over ROS1 by administering an amount of a compound or a pharmaceutical composition provided herein to said subject, wherein said amount is sufficient for selective inhibiting ALK over ROS1.

In one embodiment, provided herein is a method for selectively inhibiting ALK over TRK (e.g., TRKA, TRKB, and/or TRBC) wherein the inhibition takes place in a subject suffering from cancer, said method comprising administering an effective amount of a compound or a pharmaceutical composition provided herein to said subject. In certain embodiments, provided herein is a method of treating a subject suffering from a cancer associated with ALK, said method comprising selectively inhibiting ALK over TRK (e.g., TRKA, TRKB, and/or TRBC) by administering an amount of a compound or a pharmaceutical composition provided herein to said subject, wherein said amount is sufficient for selective inhibiting ALK over TRK (e.g., TRKA, TRKB, and/or TRBC).

As used herein and unless otherwise specified, inhibition of ALK includes inhibition of wild type ALK, or a genetically altered ALK such as a mutation, a rearrangement, or amplification or copy gain thereof, or a partially deleted ALK protein.

Cancers treated by methods of the present disclosure include, but are not limited to, lung cancer, e.g., non-small cell lung cancer, inflammatory myofibroblastic tumor, ovarian cancer, e.g., serous ovarian carcinoma, melanoma, e.g., spitzoid melanoma, glioblastoma, bile duct cancer, e.g., cholangiocarcinoma, gastric cancer, colorectal cancer, angiosarcoma, anaplastic large cell lymphoma, diffuse large B-cell lymphoma, large B-cell lymphoma, esophageal cancer, e.g., esophageal squamous cell carcinoma, kidney cancer, e.g., renal medullary carcinoma or renal cell carcinoma, breast cancer, e.g., triple negative breast cancer, thyroid cancer, e.g., papillary thyroid cancer, neuroblastoma, epithelioid hemangioendothelioma, colon cancer, and spitzoid tumor.

In one embodiment, Cancers treated by methods of the present disclosure include cancers originating from one or more oncogenic proteins selected from ROS1, ALK, TRKA, TRKB, and TRKC. In certain embodiments, cancers treated by methods of the present disclosure include cancers that are drug resistant to treatments directed at one or more oncogenic proteins selected from ROS1, ALK, TRKA, TRKB, and TRKC.

In one embodiment, the cancer in a method provided herein is anaplastic lymphoma kinase positive (ALK+). As used herein and unless otherwise specified, an “ALK positive” (ALK+) cancer, disease, or disorder refers to a cancer, disease, or disorder characterized by inappropriate (e.g., inappropriately high) expression of an ALK gene and/or the presence of a mutation in an ALK gene and/or the presence of a partially deleted ALK protein, and/or a mutation in the ALK protein, and/or is mediated by ALK, and/or that responds to inhibition of ALK. In one embodiment, “ALK positive” (ALK+) cancer, disease, or disorder refers to a cancer, disease, or disorder characterized by inappropriately high expression of an ALK gene and/or the presence of a mutation in an ALK gene, or is mediated by ALK. In one embodiment, “ALK positive” (ALK+) cancer, disease, or disorder refers to a cancer, disease, or disorder characterized by the presence of a partially deleted ALK protein (e.g., NB1, AskaSS). In one embodiment, “ALK positive” (ALK+) cancer is mediated by a genetically altered ALK. In some embodiments, the cancer, disease, or disorder carries ALK wild-type gene or genetically altered ALK gene. In one embodiment, “ALK positive” (ALK+) cancer is mediated by a fusion protein comprising a fragment of a protein encoded by an ALK gene and a fragment of a protein encoded by a gene selected from the group consisting of NPM, EML4, TPR, TFG, ATIC, CLTC1, TPM4, MSN ALO17, and MYH9. In one embodiment, the fusion protein is one or more of an EML4-ALK fusion protein, an NPM-ALK fusion protein, or a TPR-ALK fusion protein. In some embodiments, the genetically altered ALK is an EML4-ALK fusion protein. In some embodiments, the EML4-ALK fusion protein is a wild-type protein. In some embodiments, the EML4-ALK fusion protein comprises at least one resistance mutation. In some embodiments, the EML4-ALK fusion protein comprises at least one mutation selected from the group consisting of L1196M, G1202R, D1203N, L1152P/R, F1174C/L/V, C1156Y, I1171N, G1123S, S1206Y, G1269S/A, and T1151_L1152insT. In some embodiments, the EML4-ALK fusion protein comprises at least one mutation selected from the group consisting of G1202R, G1202K, L1196M, G1269A, G1269V, C1156Y, I1171T, I1171N, I1171S, F1174I, F1174L, F1174S, V1180L, S1206Y, E1129K, E1210K, T1151M, T1151_L1152insT, F1174C, G1202del, D1203N, S1206C, S1206F, L1152R, L1196Q, L1198P, L1198F, L1198H, R1275Q, L1152P, C1156T, F1245C, T1151K, I1268V, F1174V, L1198Q, S1206A, and F1245V.

In one embodiment, “ALK positive” (ALK+) cancer is mediated by a fusion protein comprising a fragment of a protein encoded by an ALK gene and a fragment of a protein encoded by a gene selected from the group consisting of NPM gene. In some embodiments, the genetically altered ALK is an NPM-ALK fusion protein. In some embodiments, the fusion protein comprises a fragment of a protein encoded by an ALK gene and a fragment of a protein encoded by a TPR gene. In some embodiments, the genetically altered ALK is a TPR-ALK fusion protein. In some embodiments, the TPR-ALK fusion protein contains a wild-type kinase domain. In some embodiments, the TPR-ALK fusion protein comprises at least one resistance mutation. In some embodiments, the TPR-ALK fusion protein comprises a L1196M mutation.

In one embodiment, the mutation alters the biological activity of an ALK nucleic acid molecule or polypeptide. As used herein and unless otherwise specified, a “mutation” or “mutant” of ALK comprises one or more deletions, substitutions, insertions, inversions, duplications, translocations, amplifications, or missense mutations, in the amino acid or nucleotide sequences of ALK, or fragments thereof. As used herein and unless otherwise specified, an ALK “rearrangement” refers to genetic translocations involving the ALK gene that may result in ALK fusion genes and/or ALK fusion proteins. The ALK fusion can also include one or more deletions, substitutions, insertions, inversions, duplications, translocations, or amplifications or a fragment thereof, as long as the mutant retains kinase phosphorylation activity.

In some embodiments, provided here is a method of treating a cancer in a subject, comprising identifying a generically altered ALK in the subject and administering to the subject a therapeutically effective amount of Compound 1 or a pharmaceutically acceptable salt thereof.

In one embodiment, the ALK mutation comprises one or more ALK point mutations. In some embodiments, cancers treated by methods of the present disclosure include one or more mutations in ALK kinase. In one embodiment, the one or more ALK point mutations are selected from point mutations at T1151, L1152, C1156, I1171, F1174, V1180, L1196, L1198, G1202, D1203, S1206, E1129, E1210, F1245, G1269, and R1275. In one embodiment, the one or more ALK point mutations is selected from R1060H, F1174C/I/L/S/V, F124SC/I/L/V, R1275L/Q, T1151M, M1166R, I1171N, I1171S, I1171N, I1183T, L1196M, A1200V, L1204F, L1240V, D1270G, Y1278S, R1192P, G1128A, G1286R, and T1343I. In one embodiment, the one or more ALK point mutations are selected from G1202R, G1202K, L1196M, G1269A, G1269V, C1156Y, I1171T, I1171N, I1171S, F1174I, F1174L, F1174S, V1180L, S1206Y, E1129K, E1210K, T1151M, T1151_L1152insT, F1174C, G1202del, D1203N, S1206Y, S1206C, S1206F, L1152R, L1196Q, L1198P, L1198F, L1198H, R1275Q, L1152P, C1156T, F1245C, T1151K, I1268V, F1174V, L1198Q, S1206A, and F1245V. In one embodiment, the ALK mutation is G1202R. In one embodiment, the ALK mutation is L1196M. In one embodiment, the ALK mutation is G1269A. In one embodiment, the ALK mutation is G1269V. In one embodiment, the ALK mutation is L1198F. In one embodiment, the ALK mutation is L1198H. In one embodiment, the ALK mutation is T1151M. In one embodiment, the ALK mutation is F1174L. In one embodiment, the ALK mutation is F1174I. In one embodiment, the ALK mutation is F1174S. In one embodiment, the ALK mutation is I1171N. In one embodiment, the ALK mutation is I1171S. In one embodiment, the ALK mutation is I1171T. In one embodiment, the ALK mutation is I1171N. In one embodiment, the ALK mutation is E1129K. In one embodiment, the ALK mutation is S1206F. In one embodiment, the ALK mutation is E1210K. In one embodiment, the ALK mutation is D1203N. In one embodiment, the ALK mutation is R1275G. In one embodiment, the ALK mutation is F1245C. In one embodiment, the ALK mutation is T1151K. In one embodiment, the ALK mutation is I1268V. In one embodiment, the ALK mutation is F1174V. In one embodiment, the ALK mutation is L1198Q. In one embodiment, the ALK mutation is S1206A.

As used herein and unless otherwise specified, a “co-mutation” refers to co-occurring mutations, i.e. when two or more mutations are present at the same time, for example in the same cell and on the same allele, in the same cell but on different alleles, or in different cells.

As used herein and unless otherwise specified, a “compound mutation” refers two or more mutations located on the same allele. A compound mutation is a subset of co-mutations. Compound mutations are also sometimes referred to as dual mutations if there are two mutations located on the same allele.

In some embodiments, the ALK mutation is co-mutation of G1202R and one or more mutations selected from L1196M, G1269A, T1151M, F1174S, and L1198F. In one embodiment, the ALK mutation is G1202R/L1196M compound mutation. In one embodiment, the ALK mutation is G1202R/G1269A compound mutation. In one embodiment, the ALK mutation is G1202R/L1198F compound mutation. In one embodiment, the ALK mutation is G1202R/T1151M compound mutation. In one embodiment, the ALK mutation is G1202R/F1174S compound mutation. In one embodiment, the ALK mutation is G1202R/F1174L compound mutation. In one embodiment, the ALK mutation is co-mutation of C1156Y and one or more mutations selected from L1256F, S1206F, F1174V, and F1174I. In one embodiment, the ALK mutation is C1156Y/L1256F compound mutation. In one embodiment, the ALK mutation is C1156Y/S1206F compound mutation. In one embodiment, the ALK mutation is C1156Y/F1174V compound mutation. In one embodiment, the ALK mutation is C1156Y/F1174I compound mutation. In one embodiment, the ALK mutation is co-mutation of L1196M and one or more mutations selected from L1198H, I1179V, and L1256F. In one embodiment, the ALK mutation is L1196M/L1198H compound mutation. In one embodiment, the ALK mutation is L1196M/I1179V compound mutation. In one embodiment, the ALK mutation is L1196M/L1256F compound mutation.

In one embodiment, the ALK mutation is G1202R/L1196M dual mutation. In one embodiment, the ALK mutation is G1202R/G1269A dual mutation. In one embodiment, the ALK mutation is G1202R/L1198F dual mutation. In one embodiment, the ALK mutation is G1202R/T1151M dual mutation. In one embodiment, the ALK mutation is G1202R/F1174S dual mutation. In one embodiment, the ALK mutation is G1202R/F1174L dual mutation. In one embodiment, the ALK mutation is C1156Y/L1256F dual mutation. In one embodiment, the ALK mutation is C1156Y/S1206F dual mutation. In one embodiment, the ALK mutation is C1156Y/F1174V dual mutation. In one embodiment, the ALK mutation is C1156Y/F1174I dual mutation. In one embodiment, the ALK mutation is L1196M/L1198H dual mutation. In one embodiment, the ALK mutation is L1196M/I1179V dual mutation. In one embodiment, the ALK mutation is L1196M/L1256F dual mutation.

In one embodiment, the ALK mutation comprises one or more ALK rearrangements (in one embodiment, one rearrangement). In one embodiment, the ALK mutation comprises one or more ALK fusions (in one embodiment, one fusion). In some embodiments, cancers treated by methods of the present disclosure include ALK fusions. In one embodiment, the ALK fusion is with one of the fusion partners described in Ou et al., JTO Clinical and Research Reports, 1(1):1-10, the entirety of which is incorporated herein by reference. In one embodiment, the ALK fusion is with one of the fusion partners selected from the group consisting of EML4, TFG, KIF5B, KLC1, STRN, HIP1, TPR, BIRC6, DCTN1, SQSTM1, SOCS5, SEC31A, CLTC, PRKAR1A, PPM1B, EIF2AK3, CRIM1, CEBPZ, PICALM, CLIP1, BCL11A, GCC2, LMO7, PHACTR1, CMTR1, VIT, DYSF, ITGAV, PLEKHA7, CUX1, VKORC1L1, FBXO36, SPTBN1, EML6, FBXO11, CLIP4, CAMKMT, NCOA1, MYT1L, SRBD1, SRD5A2, NYAP2, MPRIP, ADAM17, ALK, LPIN1, WDPCP, CEP55, ERC1, SLC16A7, TNIP2, ATAD2B, SLMAP, FBN1, SWAP70, TCF12, TRIM66, WNK3, AKAP8L, SPECC1L, PRKCB, CDK15, LCLAT1, YAP1, PLEKHM2, DCHS1, PPFIBP1, ATP13A4, C12orf75, EPAS1, FAM179A, FUT8, LIMD1, LINC00327, LOC349160, LYPD1, RBM20, TACR1, TANC1, TTC27, TUBBB, SMPD4, SORCS1, LINC00211, SOS1, C9orf3, CYBRD1, MTA3, THADA, TSPYL6, WDR37, and PLEKHH2. In one embodiment, the ALK fusion is with one of the fusion partners selected from the group consisting of EML4, TMP1, WDCP, GTF2IRD1, TPM3, TPM4, CLTC, LMNA, PRKAR1A, RANBP2, TFG, FN1, KLC1, VCL, STRN, HIP1, NPM1, DCTN1, SQSTM1, TPR, CRIM1, PTPN3, FBXO36, ATIC, MSN, ALO17, MYH9 and KIF5B. In one embodiment, the ALK mutation is EML4-ALK, a fusion between the echinoderm microtubule-associated protein-like 4 (EML4) gene and the ALK tyrosine kinase domain. There are many variants of EML4-ALK that differ by breakpoint junctions, with variant 1 (v1) and variant 3 (v3) being the most prevalent clinically. In one embodiment, the ALK mutation is NPM1-ALK. In one embodiment, the ALK mutation is STRN-ALK.

In one embodiment, the ALK mutation comprises one ALK rearrangement and one or more ALK point mutations. In one embodiment, the ALK mutation is EML4-ALK wt (variant 1). In one embodiment, the ALK mutation is EML4-ALK (variant 2). In one embodiment, the ALK mutation is EML4-ALK (variant 3). In one embodiment, the ALK mutation is EML4-ALK wt (variant 4, 5, 6, or 7). In one embodiment, the ALK mutation is EML4-ALK G1202R. In one embodiment, the ALK mutation is EML4-ALK I1171N. In one embodiment, the ALK mutation is EML4-ALK I1171S. In one embodiment, the ALK mutation is EML4-ALK I1171T. In one embodiment, the ALK mutation is EML4-ALK L1196M. In one embodiment, the ALK mutation is EML4-ALK D1203N. In one embodiment, the ALK mutation is EML4-ALK L1196M/G1202R. In one embodiment, the ALK mutation is EML4-ALK G1202R/G1269A. In one embodiment, the ALK mutation is EML4-ALK G1202R/L1196M. In one embodiment, the ALK mutation is EML4-ALK G1202R/L1198F. In one embodiment, the ALK mutation is EML4-ALK G1202R/T1151M. In one embodiment, the ALK mutation is EML4-ALK G1202R/F1174S. In one embodiment, the ALK mutation is EML4-ALK G1202R/F1174L.

In one embodiment, the ALK positive solid tumor is characterized by the presence of a mutation in an ALK gene. In one embodiment, the ALK mutation comprises one or more ALK rearrangement, one or more ALK point mutation, or a combination thereof. In one embodiment, the ALK mutation comprises G1202R, F1174C, F1174L, I1171N, I1171S, I1171T, L1196M, V1180L, C1156Y, G1202del, G1202K, G1269A, F1174S, S1206Y, E1210K, T1151M, T1151_L1152insT, D1203N, S1206C, L1152R, L1196Q, L1198P, L1198F, R1275Q, L1152P, C1156T, or F1245V, or a combination thereof. In one embodiment, the ALK mutation comprises G1202R. In one embodiment, the ALK mutation comprises F1174S or F1174L. In one embodiment, the ALK mutation comprises I1171S. In one embodiment, the ALK mutation comprises I1171T. In one embodiment, the ALK mutation comprises I1171N. In one embodiment, the ALK mutation comprises F1171M. In one embodiment, the ALK mutation comprises D1203N and one selected from I1171S, I1171T, I1171N, and I1171M. In one embodiment, the ALK mutation comprises C1156Y and one selected from I1171S, I1171T, I1171N, and I1171M. In one embodiment, the ALK mutation comprises R1275Q. In one embodiment, the ALK mutation comprises T1151M. In one embodiment, the ALK mutation comprises one or more compound mutations. In one embodiment, the compound mutation is G1202R/L1196M, G1202R/G1269A, G1202R/L1198F, or G1202R/F1174S. In one embodiment, the compound mutation is G1202R/L1196M. In one embodiment, the compound mutation is G1202R/G1269A. In one embodiment, the compound mutation is G1202R/L1198F. In one embodiment, the compound mutation is G1202R/F1174S. In one embodiment, the ALK positive solid tumor is characterized by the presence of a partially deleted ALK protein. In one embodiment, the ALK mutation is Ex2-3del. In one embodiment, the ALK mutation is Ex2-17del.

In one embodiment, partially deleted ALK proteins influence proliferative and metastatic properties of cancer cells. ALK protein can become partially deleted through various mechanisms. The first mechanism is shedding, where the 80-kDa extracellular domain of the ALK protein is post-translationally cleaved near residue Asn654, leaving the 140-kDa C-terminal transmembrane and intracellular domains on the cell. Shedding has been observed in many ALK-expressing cell lines, most notably from a neuroblastoma disease background Shedding increases cancer cell migration and proliferation in preclinical models of cancer, both in vitro and in vivo (Moog-Lutz, JBC (2005), Huang, Cell Reports (2021)). The second mechanism is alternative transcription initiation (ATI), where transcription of the ALK gene begins at an alternative initiation site downstream of the original site, resulting in the absence of exons 1-18 and part of exon 19. ALK ATIs have been identified in 11% of melanomas as well as a small portion of lung cancers and anaplastic thyroid cancers. Expression of ALK ATI transforms Ba/F3 and NIH3T3 cells, conferring them with oncogenic potential. One patient with ALK ATI has shown clinical response to an ALK inhibitor therapy, suggesting that ALK ATI may be a targetable driver mutation (Wiesner, Nature (2015)). The third mechanism is partial deletion of the ALK gene, for example through a chromosomal rearrangement event. Multiple deletion variants have been identified, including deletion of exons 2-3, exons 1-5, exons 4-11, and exons 2-17, and some of these variants have been shown to activate ALK signaling as well as transform Ba/F3 or NIH3T3 cells. ALK partial deletions have been detected in neuroblastomas, sarcomas, and lymphomas. (Okubo, Oncogene (2012); Cazes, Can Res (2013); Fransson, Genes Chromosomes & Cancer (2014); Fleuren, Can Res (2017); Fukuhara, Hematol Oncol (2017)).

In one embodiment, the ALK+ cancer is determined by an FDA-approved test or other tests known in the art. The tests that can be used include, but not limited to, e.g., FoundationOne CDx™ (F1CDx) (a sequencing based in vitro diagnostic device for detection of substitutions, insertion and deletion alterations (indels), and copy number alterations (CNAs) and selected gene rearrangements, as well as genomic signatures including microsatellite instability (MSI) and tumor mutational burden (TMB) using DNA isolated from formalin-fixed paraffin embedded (FFPE) tumor tissue specimens); VENTANA ALK (D5F3) CDx Assay (qualitative detection of the anaplastic lymphoma kinase (ALK) protein in formalin-fixed, paraffin-embedded (FFPE) non-small cell lung carcinoma (NSCLC) tissue stained with the BenchMark XT or BenchMark ULTRA automated staining instrument); and Vysis ALK Break Apart FISH Probe Kit test (a qualitative test to detect rearrangements involving the ALK gene via fluorescence in situ hybridization (FISH) in formalin-fixed, paraffin-embedded (FFPE) non-small cell lung cancer (NSCLC) tissue specimens). In one embodiment, the test is a fluorescence in situ hybridization (FISH) test, e.g., Vysis ALK Break Apart FISH Probe Kit test. Additional information for FDA-approved tests can be found at, e.g., https://www.fda.gov/MedicalDevices/ProductsandMedicalProcedures/In VitroDiagnostics/ucm303030.htm; and additional information for Vysis ALK Break Apart FISH Probe Kit can be found at, e.g., https://www.molecular.abbott/us/en/products/oncology/vysis-alk-break-apart-fish-probe-kit; the entirety of which are incorporated herein by reference.

In one embodiment of any of the methods described herein, the presence of an ALK mutation in the sample indicates that the subject has or is at increased risk for developing an ALK positive (e.g. ALK-driven) cancer. In other embodiments, the presence of the mutation in the sample indicates that the subject has or is at increased risk for developing an ALK positive (e.g. ALK-driven) cancer refractory to treatment with a TKI. In particular embodiments, the presence of the mutation in the sample indicates that the subject has or is at increased risk for developing an ALK positive (e.g. ALK-driven) cancer refractory to treatment with to one or more of crizotinib, ceritinib, alectinib, brigatinib, lorlatinib, and ASP3026. In particular embodiments, the presence of the mutation in the sample indicates that the subject has or is at increased risk for developing an ALK positive (e.g. ALK-driven) cancer refractory to treatment with to one or more of crizotinib, ceritinib, alectinib, brigatinib, and lorlatinib. In some embodiments, the ALK protein or ALK-fusion protein includes a contiguous sequence of between 30 and 1620 amino acids that has at least 95% identity to the amino acid sequence of the ALK [Homo sapiens] (NCBI Reference Sequence: NP_004295.2 or UniProt Sequence No.: Q9UM73 sequence) In another non-limiting embodiments, the ALK protein or AIK-fusion protein includes a contiguous sequence of between 30 and 1620 amino acids that has at least about 85% identity to the amino acid sequence of the ALK [Homo sapiens] (NCBI Reference Sequence: NP_004295.2 or UniProt Sequence No.: Q9UM73 sequence). In another non-limiting embodiments, where the ALK protein or ALK-fusion protein includes a contiguous sequence of between 30 and 1620 amino acids that has at least about 90% identity to the amino acid sequence of the ALK [Homo sapiens] (NCBI Reference Sequence: NP_004295.2 or UniProt Sequence No.: Q9UM73 sequence). In another non-limiting embodiments, where the ALK protein or ALK-fusion protein includes a contiguous sequence of between 30 and 1620 amino acids that has at least about 95% identity to the amino acid sequence of the ALK [Homo sapiens] (NCBI Reference Sequence: NP_004295.2 or UniProt Sequence No.: Q9UM73 sequence). In another non-limiting embodiments, where the ALK protein or ALK-fusion protein includes a contiguous sequence of between 30 and 1620 amino acids that has at least about 85-90%, 91-93%, 94%, 95%, 96%, 97%, 98%, or 99% identity to the amino acid sequence of ALK [Homo sapiens] (NCBI Reference Sequence: NP_004295.2 or UniProt Sequence No.: Q9UM73 sequence).

Also provided are methods of treating a subject having a cancer (e.g., an ALK positive cancer) that include: determining whether a cancer cell in a sample obtained from a subject having a cancer and previously administered a first ALK inhibitor, has one or more ALK inhibitor resistance mutations; and administering Compound 1 (e.g. Form 2) or a pharmaceutically acceptable salt thereof as a monotherapy or in combination with another anticancer agent to the subject if the subject has a cancer cell that has one or more ALK inhibitor resistance mutations. In some embodiments, the one or more ALK inhibitor resistance mutations confer increased resistance to a cancer cell or tumor to treatment with the first ALK inhibitor. In some embodiments, the one or more ALK inhibitor resistance mutations include one or more ALK inhibitor resistance mutations. For example, the one or more ALK inhibitor resistance mutations can include one or more point mutations at one or more of amino acid positions 1202, 1196, 1269, 1156, 1171, 1174, 1180, 1206, 1210, 1151, 1203, 1152, 1198, 1275, and 1245, e.g., G1202R, L1196M, G1269A, C1156Y, I1171T, I1171N, I1171S, F1174L, F1174S, V1180L, S1206Y, E1210K, T1151M, T1151_L1152insT, F1174C, G1202del, D1203N, S1206Y, S1206C, L1152R, L1196Q, L1198P, L1198F, R1275Q, L1152P, C1156T, and F1245V. In some embodiments, another anticancer agent is any anticancer agent known in the art. For example, another anticancer agent can be another AIK inhibitor (e.g., a second AIK inhibitor).

In one embodiment, a compound provided herein is a CNS-penetrating compound. In one embodiment, after the administration of an effective amount of a compound provided herein (e.g., orally or intravenously), the compound is able to penetrate CNS (e.g., blood-brain barrier) and achieve a concentration in CNS (e.g., brain) that is still sufficient to inhibit (e.g., selectively inhibit) ALK.

In one embodiment, provided herein is a method for treating CNS metastases of a cancer, comprising administering to a subject in need thereof an effective amount of Compound 1 or a pharmaceutically acceptable salt thereof. In one embodiment, the CNS metastases is brain metastases. In one embodiment, the cancer is an ALK-cancer.

In one embodiment, the solid tumor (or cancer) is leukocyte receptor tyrosine kinase (LTK) positive. In one embodiment, the solid tumor is LTK positive breast invasive ductal carcinoma, prostate adenocarcinoma, pancreatic adenocarcinoma, adenocarcinoma of unknown primary, or bladder urothelial carcinoma. In one embodiment, the cancer is LTK positive leukemia. In one embodiment, the solid tumor is LTK positive lung cancer. In one embodiment, the solid tumor is LTK positive NSCLC. In one embodiment, the solid tumor (or cancer) has an LTK mutation. In one embodiment, the LTK mutation is G269A, F2181, N257T, A13fs, or A214fs. In one embodiment, the solid tumor (or cancer) has an LTK fusion. In one embodiment, the LTK fusion is CLIP1-LTK. See Cooper A J, Sequist L V, Johnson T W, Lin J J. LTK fusions: A new target emerges in non-small cell lung cancer. Cancer Cell. 2022 Jan. 10; 40(1):23-25; and Izumi, H., Matsumoto, S., Liu, J. et al. The CLIP1-LTK fusion is an oncogenic driver in non-small-cell lung cancer. Nature 600, 319-323 (2021), each of which are incorporated herein by reference in their entirety.

In one embodiment, the compound is an inhibitor of human tropomyosin receptor kinase A, B, or C. In certain embodiments, the IC50 of the compound for inhibition of mutant or non-mutant ALK is no more than one-fifth of the IC50 of the compound for inhibition of wild-type tropomyosin receptor kinase A, B, or C. TRK inhibition, particularly in the central nervous system (CNS), has been associated with adverse reactions, including dizziness/ataxia/gait disturbance, paraesthesia, weight gain and cognitive changes.

In some embodiments, provided is a method of minimizing adverse events in a subject in need of treatment for cancer (e.g., an ALK positive cancer), the method comprising administering to the subject a therapeutically effective amount of Compound 1 or a pharmaceutically acceptable salt thereof, and wherein the method minimizes adverse events associated with TRK inhibitors. In some embodiments, the cancer is an AIK-associated (or ALK+) cancer. In some embodiments, the adverse events are TRK-related CNS adverse events.

As used herein “minimizing” adverse events refers to a reduction in the incidence of adverse events in a subject or patient population compared to the paradigmatic incidence of adverse events in a subject or patient population treated with TRK inhibitors (e.g., entrectinib, repotrectinib, or lorlatinib). In some embodiments, the incidence of an adverse event refers to the frequency or percentage of a specific adverse event over a subject or patient population. In some embodiments, the incidence of an adverse event refers to the total number of adverse events experienced by an individual subject. In some embodiments, minimizing adverse events refers to minimizing TRK-related CNS adverse events. In some embodiments, minimizing TRK-related CNS adverse events means less than 40% of the patient population has a TRK-related CNS adverse event. In some embodiments, minimizing TRK-related CNS adverse events means less than 35%, less than 30%, less than 25%, less than 20%, less than 15%, less than 10% or less than 5% of the patient population has a TRK-related CNS adverse event. In some embodiments, minimizing TRK-related CNS adverse events means less than 12% of the patient population have more than one TRK-related CNS adverse event. In some embodiments, minimizing TRK-related CNS adverse events means less than 11%, less than 10%, less than 9%, less than 8%, less than 7%, less than 6%, less than 5%, less than 4%, or less than 3% of the patient population have more than one TRK-related CNS adverse event.

In some embodiments, TRK-related CNS adverse events refers to one or more of the following: dizziness, ataxia, gait disturbance, paraesthesia, weight gain, hyperphagia, paresthesias, abnormal movement, cognitive changes, speech effects (e.g., dysarthria, slow speech, or speech disorder), mood disorder (e.g., irritability, anxiety, depression, affect lability, personality change, mood swings, affective disorder, aggression, agitation, mood altered, depressed mood, euphoric mood, or mania), and cognitive disorder (e.g., memory impairment, cognitive disorder, amnesia, confusion, disturbance in attention, delirium, mental impairment, attention deficit/hyperactivity disorder, dementia, sleep disturbance, or reading disorder).

In one embodiment, provided herein is a method for preventing or limiting TRK-related CNS side effect or adverse event in a cancer treatment, comprising administering to a subject in need thereof an effective amount of Compound 1 or a pharmaceutically acceptable salt thereof. In one embodiment, the method prevents the occurrence of the TRK-related CNS adverse event. In one embodiment, the method limits the frequency of occurrence of the TRK-related CNS adverse event. In one embodiment, the method limits the severity of the TRK-related side effect. In one embodiment, provided herein is a method for treating CNS metastases of a cancer with reduced TRK-related side effect, comprising administering to a subject in need thereof an effective amount of Compound 1 or a pharmaceutically acceptable salt thereof. In one embodiment, the reduction/limiting/prevention in CNS side effect or adverse event is determined in a statistical sample, as compared to a standard of care treatment, e.g., an approved ALK inhibitor (e.g., crizotinib, entrectinib, lorlatinib, or repotrectinib) for ALK+ cancer. In one embodiment, the TRK-related side effect is a TRKB-related CNS side effect. In one embodiment, the TRK-related CNS side effect or adverse event is dizziness, ataxia, gait disturbance, paraesthesia, weight gain, cognitive impairment, a mood disorder, or sleep disturbance.

In one embodiment, provided herein is a method for treating cancer, comprising administering to a subject in need thereof a therapeutically effective amount of a compound provided herein, e.g., Compound 1 (e.g. Form 2) or a pharmaceutically acceptable salt thereof. In one embodiment, the cancer is a cancer mediated by ALK. In one embodiment, the cancer is an ALK-associated cancer. In one embodiment, the cancer is an ALK+ cancer. In one embodiment, the cancer is identified to be ALK+.

In one embodiment, provided herein is a method for treating an ALK+ cancer, comprising administering to a subject in need thereof a therapeutically effective amount of Compound 1 (e.g. Form 2) or a pharmaceutically acceptable salt thereof.

In one embodiment, provided herein is a method for treating cancer in a subject, comprising: (i) identifying the cancer in the subject to be ALK+, and (ii) administering to the subject a therapeutically effective amount of Compound 1 (e.g. Form 2) or a pharmaceutically acceptable salt thereof.

In one embodiment, the cancer (or ALK+ cancer) is a solid tumor. In one embodiment, the cancer (or ALK+ cancer) is lung cancer, e.g., non-small cell lung cancer (NSCLC), glioblastoma, inflammatory myofibroblastic tumor (IMT), bile duct cancer, e.g., cholangiocarcinoma, ovarian cancer, e.g., serous ovarian carcinoma, gastric cancer, colorectal cancer, angiosarcoma, melanoma, e.g., spitzoid melanoma, epithelioid hemangioendothelioma, esophageal cancer, e.g., esophageal squamous cell carcinoma (ESCC), kidney cancer, e.g., renal medullary carcinoma or renal cell carcinoma, breast cancer, e.g., triple negative breast cancer, colon cancer, thyroid cancer, e.g., papillary thyroid cancer, spitzoid tumor, pancreatic cancer, inflammatory hepatocellular adenoma, or neuroblastoma. In one embodiment, the cancer (or ALK+ cancer) is ALCL, NSCLC, neuroblastoma, inflammatory myofibroblastic tumor, adult renal cell carcinoma, pediatric renal cell carcinoma, breast cancer, ER⁺ breast cancer, colonic adenocarcinoma, glioblastoma, glioblastoma multiforme, anaplastic thyroid cancer, cholangiocarcinoma, ovarian cancer, gastric adenocarcinoma, colorectal cancer, inflammatory myofibroblastic tumor, angiosarcoma, epithelioid hemangioendothelioma, intrahepatic cholangiocarcinoma, thyroid papillary cancer, spitzoid neoplasms, sarcoma, astrocytoma, brain lower grade glioma, secretory breast carcinoma, mammary analogue carcinoma, acute myeloid leukemia, congenital mesoblastic nephroma, congenital fibrosarcomas. Ph-like acute lymphoblastic leukemia, thyroid carcinoma, skin cutaneous melanoma, head and neck squamous cell carcinoma, pediatric glioma CML, prostate cancer, lung squamous carcinoma, ovarian serous cystadenocarcinoma, skin cutaneous melanoma, castrate-resistant prostate cancer. Hodgkin lymphoma, and serous and clear cell endometrial cancer.

In one embodiment, the cancer is lung cancer. In one embodiment, the cancer is non-small cell lung cancer. In one embodiment, the cancer is advanced or metastatic non-small cell lung cancer. In one embodiment, the cancer is ALK+ non-small cell lung cancer. In one embodiment, the cancer is relapsed or refractory non-small cell lung cancer. In one embodiment, the cancer is relapsed or refractory ALK+ non-small cell lung cancer. In one embodiment, the cancer is newly diagnosed non-small cell lung cancer. In one embodiment, the cancer is newly diagnosed ALK+ non-small cell lung cancer.

In one embodiment, the cancer is glioblastoma. In one embodiment, the cancer is relapsed or refractory glioblastoma. In one embodiment, the cancer is relapsed or refractory ALK+ glioblastoma. In one embodiment, the cancer is newly diagnosed glioblastoma. In one embodiment, the cancer is newly diagnosed ALK+ glioblastoma.

In one embodiment, the cancer is IMT. In one embodiment, the cancer is ALK+ IMT. In one embodiment, the cancer is relapsed or refractory IMT. In one embodiment, the cancer is relapsed or refractory ALK+ IMT. In one embodiment, the cancer is newly diagnosed IMT. In one embodiment, the cancer is newly diagnosed ALK+ IMT.

In one embodiment, the cancer is bile duct cancer. In one embodiment, the cancer is cholangiocarcinoma. In one embodiment, the cancer is ALK+ cholangiocarcinoma. In one embodiment, the cancer is relapsed or refractory cholangiocarcinoma. In one embodiment, the cancer is relapsed or refractory ALK+ cholangiocarcinoma. In one embodiment, the cancer is newly diagnosed cholangiocarcinoma. In one embodiment, the cancer is newly diagnosed ALK+ cholangiocarcinoma. In one embodiment, the cholangiocarcinoma patient has STRN-ALK fusion mutation. In one embodiment, the cholangiocarcinoma patient has progressed on alectinib treatment. In one embodiment, the cholangiocarcinoma patient has acquired G1202R resistance mutation.

In one embodiment, the cancer is ovarian cancer. In one embodiment, the cancer is ALK+ ovarian cancer. In one embodiment, the cancer is relapsed or refractory ovarian cancer. In one embodiment, the cancer is relapsed or refractory ALK+ ovarian cancer. In one embodiment, the cancer is newly diagnosed ovarian cancer. In one embodiment, the cancer is newly diagnosed ALK+ ovarian cancer. In one embodiment, the ovarian cancer is serous ovarian carcinoma. In one embodiment, the ovarian cancer is high grade serous ovarian carcinoma.

In one embodiment, the cancer is gastric cancer. In one embodiment, the cancer is ALK+ gastric cancer. In one embodiment, the cancer is relapsed or refractory gastric cancer. In one embodiment, the cancer is relapsed or refractory ALK+ gastric cancer. In one embodiment, the cancer is newly diagnosed gastric cancer. In one embodiment, the cancer is newly diagnosed ALK+ gastric cancer.

In one embodiment, the cancer is colorectal cancer. In one embodiment, the cancer is ALK+ colorectal cancer. In one embodiment, the cancer is relapsed or refractory colorectal cancer. In one embodiment, the cancer is relapsed or refractory ALK+ colorectal cancer. In one embodiment, the cancer is newly diagnosed colorectal cancer. In one embodiment, the cancer is newly diagnosed ALK+ colorectal cancer.

In one embodiment, the cancer is angiosarcoma. In one embodiment, the cancer is ALK+ angiosarcoma. In one embodiment, the cancer is relapsed or refractory angiosarcoma. In one embodiment, the cancer is relapsed or refractory ALK+ angiosarcoma. In one embodiment, the cancer is newly diagnosed angiosarcoma. In one embodiment, the cancer is newly diagnosed ALK+ angiosarcoma.

In one embodiment, the cancer is sarcoma. In one embodiment, the cancer is soft-tissue sarcoma. In one embodiment, the cancer is synovial sarcoma. In one embodiment, the cancer is one or more selected from the group consisting of inflammatory myofibroblastic tumor, Leiomyosarcoma, and neurofibroma. In one embodiment, the cancer is one or more selected from the group consisting of Ewing sarcoma, fibrosarcoma, osteosarcoma, pulmonary sarcoma, uterine carcinosarcoma, and uterine leiomyosarcoma.

In one embodiment, the cancer is melanoma. In one embodiment, the cancer is spitzoid tumor. In one embodiment, the cancer is spitzoid melanoma. In one embodiment, the cancer is ALK+ spitzoid melanoma. In one embodiment, the cancer is relapsed or refractory spitzoid melanoma. In one embodiment, the cancer is relapsed or refractory ALK+ spitzoid melanoma. In one embodiment, the cancer is newly diagnosed spitzoid melanoma. In one embodiment, the cancer is newly diagnosed ALK+ spitzoid melanoma.

In one embodiment, the cancer is epithelioid hemangioendothelioma. In one embodiment, the cancer is ALK+ epithelioid hemangioendothelioma. In one embodiment, the cancer is relapsed or refractory epithelioid hemangioendothelioma. In one embodiment, the cancer is relapsed or refractory ALK+ epithelioid hemangioendothelioma. In one embodiment, the cancer is newly diagnosed epithelioid hemangioendothelioma. In one embodiment, the cancer is newly diagnosed ALK+ epithelioid hemangioendothelioma.

In one embodiment, the cancer is esophageal cancer. In one embodiment, the cancer is ESCC. In one embodiment, the cancer is ALK+ ESCC. In one embodiment, the cancer is relapsed or refractory ESCC. In one embodiment, the cancer is relapsed or refractory ALK+ ESCC. In one embodiment, the cancer is newly diagnosed ESCC. In one embodiment, the cancer is newly diagnosed ALK+ ESCC.

In one embodiment, the cancer is kidney cancer. In one embodiment, the cancer is renal medullary carcinoma. In one embodiment, the cancer is ALK+ renal medullary carcinoma. In one embodiment, the cancer is relapsed or refractory renal medullary carcinoma. In one embodiment, the cancer is relapsed or refractory ALK+ renal medullary carcinoma. In one embodiment, the cancer is newly diagnosed renal medullary carcinoma. In one embodiment, the cancer is newly diagnosed ALK+ renal medullary carcinoma. In one embodiment, the cancer is renal cell carcinoma. In one embodiment, the cancer is ALK+ renal cell carcinoma. In one embodiment, the cancer is relapsed or refractory renal cell carcinoma. In one embodiment, the cancer is relapsed or refractory ALK+ renal cell carcinoma. In one embodiment, the cancer is newly diagnosed renal cell carcinoma. In one embodiment, the cancer is newly diagnosed ALK+ renal cell carcinoma.

In one embodiment, the cancer is breast cancer. In one embodiment, the cancer is ALK+ breast cancer. In one embodiment, the cancer is relapsed or refractory breast cancer. In one embodiment, the cancer is relapsed or refractory ALK+ breast cancer. In one embodiment, the cancer is newly diagnosed breast cancer. In one embodiment, the cancer is newly diagnosed ALK+ breast cancer. In one embodiment, the breast cancer is triple negative breast cancer.

In one embodiment, the cancer is colon cancer. In one embodiment, the cancer is ALK+ colon cancer. In one embodiment, the cancer is relapsed or refractory colon cancer. In one embodiment, the cancer is relapsed or refractory ALK+ colon cancer. In one embodiment, the cancer is newly diagnosed colon cancer. In one embodiment, the cancer is newly diagnosed ALK+ colon cancer.

In one embodiment, the cancer is thyroid cancer. In one embodiment, the cancer is papillary thyroid cancer. In one embodiment, the cancer is ALK+ papillary thyroid cancer. In one embodiment, the cancer is relapsed or refractory papillary thyroid cancer. In one embodiment, the cancer is relapsed or refractory ALK+ papillary thyroid cancer. In one embodiment, the cancer is newly diagnosed papillary thyroid cancer. In one embodiment, the cancer is newly diagnosed ALK+ papillary thyroid cancer.

In one embodiment, the cancer is ALK+ glioma (e.g. Grade 1, Grade 2, Grade 3, or Grade 4). In one embodiment, the cancer is relapsed or refractory glioma. In one embodiment, the cancer is relapsed or refractory ALK+ glioma. In one embodiment, the cancer is newly diagnosed ALK+ glioma. In one embodiment, the cancer is ALK+ glioblastoma. In one embodiment, the cancer is newly diagnosed ALK+ glioblastoma. In one embodiment, the cancer is relapsed or refractory glioblastoma. In one embodiment, the cancer is relapsed or refractory ALK+ glioblastoma. In one embodiment, the cancer is neuroblastoma. In one embodiment, the cancer is ALK+ neuroblastoma. In one embodiment, the cancer is relapsed or refractory neuroblastoma. In one embodiment, the cancer is relapsed or refractory ALK+ neuroblastoma. In one embodiment, the cancer is newly diagnosed neuroblastoma. In one embodiment, the cancer is newly diagnosed ALK+ neuroblastoma.

In one embodiment, the cancer is ALK+ pancreatic cancer. In one embodiment, the cancer is relapsed or refractory pancreatic cancer. In one embodiment, the cancer is relapsed or refractory ALK+ pancreatic cancer. one embodiment, the cancer is newly diagnosed neuroblastoma. In one embodiment, the cancer is newly diagnosed ALK+ pancreatic cancer.

In one embodiment, the cancer is ALK+ inflammatory hepatocellular adenoma. In one embodiment, the cancer is relapsed or refractory inflammatory hepatocellular adenoma. In one embodiment, the cancer is relapsed or refractory ALK+ inflammatory hepatocellular adenoma. In one embodiment, the cancer is newly diagnosed neuroblastoma. In one embodiment, the cancer is newly diagnosed ALK+ inflammatory hepatocellular adenoma.

In one embodiment, the cancer is hematological cancer. In one embodiment, the cancer is ALK+ hematological cancer.

In one embodiment, the cancer is ALK+ lymphoma. In one embodiment, the lymphoma is non-Hodgkin lymphoma. In one embodiment, the lymphoma is ALK+ non-Hodgkin lymphoma. In one embodiment, the lymphoma is ALK+ anaplastic large cell lymphoma (ALCL), diffuse large B-cell lymphoma (DLBCL), or large B-cell lymphoma.

In one embodiment, the cancer is ALCL. In one embodiment, the cancer is ALK+ ALCL. In one embodiment, the cancer is relapsed or refractory ALCL. In one embodiment, the cancer is relapsed or refractory ALK+ ALCL. In one embodiment, the cancer is relapsed or refractory ALK+ ALCL. In one embodiment, the cancer is newly diagnosed ALCL. In one embodiment, the cancer is newly diagnosed ALK+ ALCL.

In one embodiment, the cancer is DLBCL. In one embodiment, the cancer is ALK+ DLBCL. In one embodiment, the cancer is relapsed or refractory DLBCL. In one embodiment, the cancer is relapsed or refractory ALK+ DLBCL. In one embodiment, the cancer is newly diagnosed DLBCL. In one embodiment, the cancer is newly diagnosed ALK+ DLBCL.

In one embodiment, the cancer is large B-cell lymphoma. In one embodiment, the cancer is ALK+ large B-cell lymphoma. In one embodiment, the cancer is relapsed or refractory large B-cell lymphoma. In one embodiment, the cancer is relapsed or refractory ALK+ large B-cell lymphoma. In one embodiment, the cancer is newly diagnosed large B-cell lymphoma. In one embodiment, the cancer is newly diagnosed ALK+ large B-cell lymphoma. In one embodiment, the cancer (or ALK+ cancer) is new diagnosed. In one embodiment, the cancer (or ALK+ cancer) is previously untreated.

In one embodiment, the cancer is one or more selected from the group consisting of acinar adenocarcinoma, adrenocortical carcinoma, anaplastic astrocytoma, anaplastic large cell lymphoma, B-cell acute lymphocytic leukemia, B-cell lymphoma, breast cancer, cervical squamous cell carcinoma, chromophobe renal cell carcinoma, clear cell renal cell carcinoma, colorectal adenocarcinoma, cutaneous melanoma, diffuse large B-cell lymphoma, diffuse-type gastric cancer, endocervical adenocarcinoma, endometrial adenocarcinoma, epithelial ovarian cancer, esophageal cancer, Ewing sarcoma, fallopian tube serous carcinoma, fibrosarcoma. gallbladder carcinoma, ganglioglioma, gastroesophageal junction adenocarcinoma, glioblastoma, head and neck cancer, head and neck squamous cell carcinoma, hepatocellular carcinoma, high-grade serous carcinoma, inflammatory myofibroblastic tumor, leiomyosarcoma, neurofibroma, soft tissue sarcoma, invasive ductal carcinoma, low-grade serous carcinoma, lung adenocarcinoma, medulloblastoma, melanoma, mucinous cystadenocarcinoma, mucoepidermoid carcinoma, mucoepidermoid carcinoma, multiple myeloma, neuroblastoma, non-Hodgkins lymphoma, osteosarcoma, ovarian serous cystadenocarcinoma, pancreatic adenocarcinoma, pancreatic cancer, pancreatic ductal adenocarcinoma, papillary cell renal cell carcinoma, papillary thyroid carcinoma, pericardial mesothelioma, peritoneal mesothelioma, prostate adenocarcinoma, pulmonary sarcoma, renal cell carcinoma, salivary gland cancer, small cell lung carcinoma, small intestine cancer, smooth muscle tumor of uncertain malignant potential, squamous cell carcinoma, stomach cancer, thyroid cancer, urothelial carcinoma, uterine carcinosarcoma, uterine corpus endometrial carcinoma, uterine leiomyosarcoma, uterine papillary serous carcinoma, and uterine sarcoma.

In one embodiment, the cancer (or ALK+ cancer) is newly diagnosed. In one embodiment, the cancer (or ALK+ cancer) is previously untreated.

In one embodiment, the cancer (or ALK+ cancer) is relapsed or refractory. In one embodiment, the cancer is relapsed. In one embodiment, the cancer (or ALK+ cancer) is refractory. In one embodiment, the cancer (or ALK+ cancer) is refractory to an inhibitor of wild-type ALK. In one embodiment, the cancer (or ALK+ cancer) is refractory to an inhibitor of genetically altered ALK. In one embodiment, the cancer (or ALK+ cancer) is refractory to one or more of crizotinib, ceritinib, alectinib, brigatinib, and lorlatinib. In one embodiment, the cancer (or ALK+ cancer) is refractory to one or more of crizotinib, ceritinib, alectinib, and brigatinib. In one embodiment, the cancer (or ALK+ cancer) is refractory to one or more of crizotinib, ceritinib, alectinib, brigatinib, ASP3026, and lorlatinib.

In one embodiment, the cancer (or ALK+ cancer) is intolerable to one or more of crizotinib, ceritinib, alectinib, brigatinib, and lorlatinib. In one embodiment, the cancer (or ALK+ cancer) is intolerable to one or more of crizotinib, ceritinib, alectinib, and brigatinib. In one embodiment, the cancer (or ALK+ cancer) is intolerable to one or more of crizotinib, ceritinib, alectinib, brigatinib, ASP3026 and lorlatinib. As used herein, the term “intolerant to” a drug refers to a subject (e.g. a patient) who must discontinue or suspend treatment with a drug because of unacceptable side effects. The subject (e.g. patient) can be intolerant either from the initial administration of the therapy or become intolerant over time in response to the therapy.

In one embodiment, the subject is previously untreated. In one embodiment, the subject is treatment naïve to tyrosine kinase inhibitor (TKI) therapy. In one embodiment, the subject has received one or more prior lines of therapy. In one embodiment, the subject has received two or more prior lines of therapy. In one embodiment, the subject has developed resistance to one or more of the prior lines of therapy. In one embodiment, the subject has progressed on one or more of the prior lines of therapy. In one embodiment, the prior therapy comprises a tyrosine kinase inhibitor (TKI). In one embodiment, the prior TKI therapy comprises a treatment with one or more of crizotinib, ceritinib, alectinib, brigatinib, lorlatinib, entrectinib, repotrectinib, cabozantinib, foretinib, taletrectinib, merestinib, masitinib, and ensartinib. In one embodiment, the subject has progressed on the prior treatment of crizotinib, alectinib, ceritinib, brigatinib, or lorlatinib. In one embodiment, the subject has progressed on the prior treatment of crizotinib, alectinib, ceritinib, brigatinib, or lorlatinib, and at least one other TKI. In one embodiment, the subject has progressed on the prior treatment of crizotinib. In one embodiment, the subject has progressed on the prior treatment of alectinib. In one embodiment, the subject has progressed on the prior treatment of ceritinib. In one embodiment, the subject has progressed on the prior treatment of brigatinib. In one embodiment, the subject has progressed on the prior treatment of lorlatinib. In one embodiment, the prior therapy comprises one or more chemotherapies. In one embodiment, the subject has progressed on the prior treatment of TKI (e.g. one or more from crizotinib, alectinib, ceritinib, brigatinib, and lorlatinib) and one or more chemotherapies. In one embodiment, the prior therapy comprises two or more chemotherapies. In one embodiment, the prior therapy comprises one or more immunotherapies. In one embodiment, the subject has progressed on the prior treatment of TKI (e.g. one or more from crizotinib, alectinib, ceritinib, brigatinib, and lorlatinib) and one or more immunotherapies. In one embodiment, the prior therapy comprises two or more immunotherapies. In one embodiment, the one or more chemotherapies and/or one or more immunotherapies are in addition to the TKI therapy.

In one embodiment, the cancer is advanced cancer, e.g. relapsed after, refractory to, progressed on, or resistant to the prior treatment by a TKI. In some embodiments, the cancer is locally advanced.

In one embodiment, the cancer (or ALK+ cancer) is resistant to a tyrosine kinase inhibitor (TKI). In one embodiment, the cancer (or ALK+ cancer) is resistant to a prior tyrosine kinase inhibitor (TKI).

In one embodiment, the cancer is resistant lung cancer. In one embodiment, the cancer is resistant non-small cell lung cancer. In one embodiment, the cancer is non-small cell lung cancer resistant to a TKI. In one embodiment, the cancer is ALK+ non-small cell lung cancer resistant to a TKI.

In one embodiment, the cancer is lung cancer (e.g., NSCLC). In one embodiment, the cancer is advanced lung cancer, e.g. relapsed after, or refractory to, prior treatment by a TKI.

In one embodiment, a compound provided herein is administered as first-line treatment. In one embodiment, a compound provided herein is administered as second-line treatment. In one embodiment, a compound provided herein is administered as third or fourth-line treatment.

In one embodiment, the cancer (or ALK cancer) is metastatic. In one embodiment, the cancer has CNS metastases. In one embodiment, the cancer has brain metastases. In one embodiment, the cancer is metastatic non-small cell lung cancer (NSCLC). In one embodiment, the cancer is metastatic ALK+ NSCLC.

In one embodiment, provided herein is a method for treating a subject with metastatic ALK+ non-small cell lung cancer (NSCLC), comprising administering to the subject a therapeutically effective amount of a compound provided herein, e.g., Compound 1 (e.g. Form 2), or a pharmaceutically acceptable salt thereof.

In one embodiment, the subject is an adult subject of more than 18 years old. In one embodiment, the subject is a pediatric subject of no more than 18 years old. In one embodiment, the subject is a pediatric subject of at least 12 years old but no more than 18 years old. In one embodiment, the subject is a patient.

In one embodiment, provided herein is a method for treating an adult subject with metastatic ALK+ NSCLC, comprising administering to the subject a therapeutically effective amount of a compound provided herein, e.g., Compound 1 (e.g. Form 2), or a pharmaceutically acceptable salt thereof.

In one embodiment, provided herein is a method for treating an adult subject with metastatic ALK+ NSCLC, comprising administering to the subject a therapeutically effective amount of Compound 1 or a pharmaceutically acceptable salt thereof, wherein the subject has progressed on or is intolerant of at least 1 prior TKI therapy.

In one embodiment, provided herein is a method for treating an ALK-associated (or ALK+) cancer in a subject in need thereof, wherein the cancer has developed resistance to a tyrosine kinase inhibitor (TKI), the method comprising administering to the subject a therapeutically effective amount of a compound provided herein, e.g., Compound 1 (e.g. Form 2), or a pharmaceutically acceptable salt thereof.

In one embodiment, provided herein is a method for treating an ALK-associated (e.g. ALK+) cancer in a subject in need thereof, wherein the cancer has developed resistance to a tyrosine kinase inhibitor (TKI), and wherein the cancer has been identified as having one or more ALK inhibitor resistance mutations, the method comprising administering o the subject a therapeutically effective amount of a compound provided herein, e.g., Compound 1 (e.g. Form 2), or a pharmaceutically acceptable salt thereof. In one embodiment, the one or more ALK inhibitor resistance mutations comprise one or more amino acid substitutions at an amino acid position selected from 1151, 1196, 1198, 1202, and 1269. In one embodiment, the one or more ALK inhibitor resistance mutations comprise one or more amino acid substitutions selected from T1151M, L1196M, L1198F, G1202R, and G1269A. In one embodiment, the one or more ALK inhibitor resistance mutations is G1202R. In one embodiment, the one or more ALK inhibitor resistance mutations comprise G1202R and one or more of L1196M, L1198F, G1269A, and T1151M.

In one embodiment, provided herein is a method for treating an adult subject with metastatic NSCLC that is ALK+ with mutation G1202R, comprising administering to the subject a therapeutically effective amount of a compound provided herein, e.g., Compound 1 (e.g. Form 2), or a pharmaceutically acceptable salt thereof, wherein the subject has progressed on or is intolerant of at least 1 prior TKI therapy.

In one embodiment, provided herein is a method for treating an ALK-associated (e.g. ALK+) cancer in a subject in need thereof, wherein the cancer has developed resistance to a tyrosine kinase inhibitor (TKI), the method comprising administering to the subject a therapeutically effective amount of a compound provided herein, e.g., Compound 1 (e.g. Form 2), or a pharmaceutically acceptable salt thereof.

In one embodiment, the cancer or disease is in a pediatric subject (including an infantile subject). In one embodiment, the cancer is systemic anaplastic large cell lymphoma (ALCL) that is ALK+ in pediatric subjects 1 year of age or older, and young adults. In another embodiment, the cancer is relapsed or refractory systemic anaplastic large cell lymphoma (ALCL) that is ALK+ in pediatric subjects 1 year of age or older, and young adults.

In one embodiment, the TKI is an ALK inhibitor. In one embodiment, the TKI is crizotinib, ceritinib, alectinib, brigatinib, lorlatinib, entrectinib, repotrectinib, cabozantinib, foretinib, merestinib, taletrectinib, masitinib, or ensartinib. In one embodiment, the TKI is crizotinib. In one embodiment, the TKI is ceritinib. In one embodiment, the TKI is alectinib. In one embodiment, the TKI is brigatinib. In one embodiment, the TKI is lorlatinib. In one embodiment, the TKI is a first generation ALK TKI (e.g., crizotinib). In one embodiment, the TKI is a second generation ALK TKI (e.g., ceritinib, alectinib, or brigatinib). In one embodiment, the TKI is a third generation ALK TKI (e.g., lorlatinib).

In certain embodiments, the subject has relapsed after the first-line treatment of the cancer. In certain embodiments, the subject has relapsed after the first-line treatment of the cancer with one ALK TKI selected from crizotinib, ceritinib, alectinib, and brigatinib. In certain embodiments, the subject has relapsed after the treatment of the cancer with at least one ALK TKI selected from crizotinib, ceritinib, alectinib, brigatinib, and lorlatinib. In other embodiments, the subject has relapsed after the second-line treatment of the cancer. In other embodiments, the subject has relapsed after the second-line treatment of the cancer with one or more ALK TKI selected from crizotinib, ceritinib, alectinib, brigatinib, and lorlatinib. In other embodiments, the subject has relapsed after the third-line treatment of the cancer. In other embodiments, the subject has relapsed after second-line treatment or the third-line treatment of the cancer with lorlatinib in the second or third line.

In certain embodiments, the subject has developed resistance after the first-line treatment of the cancer. In certain embodiments, the subject has developed resistance after the first-line treatment of the cancer with one ALK TKI selected from crizotinib, ceritinib, alectinib, and brigatinib. In certain embodiments, the subject has developed resistance after the treatment of the cancer with at least one ALK TKI selected from crizotinib, ceritinib, alectinib, brigatinib, and lorlatinib. In other embodiments, the subject has developed resistance after the second-line treatment of the cancer. In other embodiments, the subject has developed resistance after the second-line treatment of the cancer with one or more ALK TKI selected from crizotinib, ceritinib, alectinib, brigatinib, and lorlatinib. In other embodiments, the subject has developed resistance after the third-line treatment of the cancer. In other embodiments, the subject has developed resistance after the second-line treatment or third-line treatment of the cancer with lorlatinib in the second or third line.

In one embodiment, Compound 1 is administered to the patient QD for one or more days. In one embodiment, Compound 1 is administered to the patient BID for one or more days. In one embodiment, Compound 1 is administered to the patient QD for at least one treatment cycle. In one embodiment, Compound 1 is administered to the patient BID for at least one treatment cycle. In one embodiment, one treatment cycle is at least 7 days. In one embodiment, one treatment cycle is at least 14 days. In one embodiment, one treatment cycle is at least 15 days. In one embodiment, one treatment cycle is at least 21 days. In one embodiment, one treatment cycle is at least 28 days. In one embodiment, Compound 1 is administered to the patient for at least one treatment cycle (e.g. 21 days). In one embodiment, Compound 1 is administered to the patient for at least two treatment cycles (e.g. 42 days). In one embodiment, Compound 1 is administered to the patient for at least three treatment cycles (e.g. 63 days).

In one embodiment, the patient does not experience a Grade 4 adverse event after the administration of Compound 1. In one embodiment, the patient does not experience a Grade 3 adverse event after the administration of Compound 1. In one embodiment, the patient does not experience a Grade 2 adverse event after the administration of Compound 1. In one embodiment, the patient does not experience a Grade 1 adverse event after the administration of Compound 1. As used here and unless otherwise specified, the grade of the adverse event follows the National Cancer Institute Common Terminology Criteria for Adverse Events (CTCAE) grades, which is incorporated here by reference. In some embodiments, the adverse event is TEAE. In some embodiments, the adverse event is TRAE.

In one embodiment, the patient does not experience a neurologic adverse event after the administration of Compound 1. In one embodiment, the patient does not experience a psychiatric event (e.g. insomnia) after the administration of Compound 1. In one embodiment, the patient does not experience one or more CNS adverse event selected from the group consisting of dizziness, ataxia, gait disturbance, paraesthesia, weight gain, hyperphagia, paresthesias, abnormal movement, cognitive changes, seizures, hallucinations, speech disorders (e.g., aphasia, dysarthria, slow speech, or speech disorder), mood disorder (e.g., irritability, anxiety, depression, affective disorder, affect lability, personality change, mood swings, affective disorder, aggression, stress, agitation, mood altered, depressed mood, euphoric mood, suicidal ideation, or mania), mental status, sleep disorder, and cognitive disorder (e.g., memory impairment, cognitive disorder, amnesia, confusion, disturbance in attention, delirium, mental impairment, attention deficit/hyperactivity disorder, dementia, sleep disturbance, disorientation, or reading disorder). In one embodiment, the neurologic adverse event is one or more selected from the group consisting of cognitive impairment, mood disorders, sleep disturbances, dizziness, and ataxia. In one embodiment, the patient does not experience an adverse event of weight gain and/or glucose metabolism disorders. In one embodiment, the glucose metabolism disorder is hyperglycemia (e.g., diabetes). In one embodiment, the glucose metabolism disorder is hypoglycemia. In one embodiment, the patient experiences a Grade 1 treatment-related adverse event (TRAE) after the administration of Compound 1. In one embodiment, the patient experiences at most a Grade 1 TRAE after the administration of Compound 1. In one embodiment, the patient experiences a Grade 2 TRAE after the administration of Compound 1. In one embodiment, the patient experiences at most a Grade 2 TRAE after the administration of Compound 1. In one embodiment, the patient experiences a Grade 3 TRAE after the administration of Compound 1. In one embodiment, the patient experiences at most a Grade 3 TRAE after the administration of Compound 1. In one embodiment, the patient experiences a Grade 1 treatment emergent adverse event (TEAE) after the administration of Compound 1. In one embodiment, the patient experiences at most a Grade 1 TEAE after the administration of Compound 1. In one embodiment, the patient experiences a Grade 2 TEAE after the administration of Compound 1. In one embodiment, the patient experiences at most a Grade 2 TEAE after the administration of Compound 1. In one embodiment, the patient experiences a Grade 3 TEAE after the administration of Compound 1. In one embodiment, the patient experiences at most a Grade 3 TEAE after the administration of Compound 1.

In one embodiment, the patient does not experience substantial hepatotoxicity after the administration of Compound 1 or a pharmaceutically acceptable salt. In some embodiments, the patient does not experience substantial elevation of a transaminase after the administration of Compound 1 or a pharmaceutically acceptable salt. In some embodiments, the patient does not experience substantial elevation of alanine aminotransferase (ALT) after the administration of Compound 1 or a pharmaceutically acceptable salt. In some embodiments, the patient at most experiences Grade 3 elevation of ALT as the treatment-emergent adverse event (TEAE) or treatment-related adverse event (TRAE) after the administration of Compound 1 or a pharmaceutically acceptable salt. In some embodiments, the patient at most experiences Grade 2 elevation of ALT as the TEAE or TRAE after the administration of Compound 1 or a pharmaceutically acceptable salt. In some embodiments, the patient at most experiences Grade 1 elevation of ALT as the TEAE or TRAE after the administration of Compound 1 or a pharmaceutically acceptable salt. In some embodiments, the patient does not experience substantial elevation of aspartate aminotransferase (AST) after the administration of Compound 1 or a pharmaceutically acceptable salt. In some embodiments, the patient at most experiences Grade 3 elevation of AST as the TEAE or TRAE after the administration of Compound 1 or a pharmaceutically acceptable salt. In some embodiments, the patient at most experiences Grade 2 elevation of AST as the TEAE or TRAE after the administration of Compound 1 or a pharmaceutically acceptable salt thereof. In some embodiments, the patient at most experiences Grade 1 elevation of AST as the TEAE or TRAE after the administration of Compound 1 or a pharmaceutically acceptable salt thereof. In some embodiments, the ALT or AST elevation occurs within about two months after administration of Compound 1 or a pharmaceutically acceptable salt thereof and returns to the normal limits or baseline afterwards. In some embodiments, the ALT or AST elevation occurs within about two months after administration of Compound 1 or a pharmaceutically acceptable salt thereof and returns to the normal limits or baseline within about 3 months after the occurrence of the ALT or AST elevation. In some embodiments, the ALT or AST elevation occurs within about two months after administration of Compound 1 or a pharmaceutically acceptable salt thereof and returns to the normal limits or baseline within about 2 months after the occurrence of the ALT or AST elevation. In some embodiments, the ALT or AST elevation occurs within about two months after administration of Compound 1 or a pharmaceutically acceptable salt thereof and returns to the normal limits or baseline within about one month after the occurrence of the ALT or AST elevation. In some embodiments, the ALT or AST elevation occurs within about two months after administration of Compound 1 or a pharmaceutically acceptable salt thereof and returns to the normal limits or baseline within about three weeks after the occurrence of the ALT or AST elevation. In some embodiments, the ALT or AST elevation occurs within about two months after administration of Compound 1 or a pharmaceutically acceptable salt thereof and returns to the normal limits or baseline within about two weeks after the occurrence of the ALT or AST elevation. In one embodiment, the patient does not experience substantial increase of ALT or AST after the administration of Compound 1 or a pharmaceutically acceptable salt thereof. In some embodiments, the administration is once daily. In some embodiments, the administration is twice daily. In some embodiments, the administration is once daily continuously for the dosing period. In some embodiments, the administration is twice daily continuously for the dosing period.

In some embodiments, after Compound 1 or a pharmaceutically acceptable salt thereof is administered at a dose level to a patient, the patient experiences Grade 1 increase of ALT or AST as TEAE or TRAE and the administration continues at the same dose level. In some embodiments, after Compound 1 or a pharmaceutically acceptable salt thereof is administered at a dose level to a patient, the patient experiences Grade 2 increase of ALT or AST as TEAE or TRAE and the administration continues at the same dose level (in a further embodiment, under weekly monitoring (e.g. until the increase of ALT or AST returns to Grade 1 or lower)). In some embodiments, after Compound 1 or a pharmaceutically acceptable salt thereof is administered at a dose level to a patient, the patient experiences Grade 3 increase of ALT or AST as TEAE or TRAE and the administration is subject to dose interruption (i.e., the administration is stopped/withheld for a period of time). In some embodiments, after Compound 1 or a pharmaceutically acceptable salt thereof is administered at a dose level to a patient, the patient experiences Grade 3 increase of ALT or AST as TEAE or TRAE and the administration is subject to dose modification (e.g. reduction). In some embodiments, the patient resumes the same dose level after the dose interruption when the increase of ALT or AST returns to Grade 1 or lower, or the ALT or AST returns to baseline. In some embodiments, the patient resumes the same dose level (the first dose level) after the dose interruption and experiences the same grade of increase of ALT or AST (which in one embodiment resolves (e.g. severity returns to Grade 1 or lower, or ALT or AST returns to baseline) within 30 days), then the patient is subject to a lower dose level (the second dose level, e.g. according to Table A). In one embodiment, the first dose level is 50 mg QD, and the second dose level is 25 mg QD. In one embodiment, the first dose level is 100 mg QD, and the second dose level is 50 mg QD. In one embodiment, the first dose level is 150 mg QD, and the second dose level is 100 mg QD. In one embodiment, the first dose level is 200 mg QD, and the second dose level is 150 mg QD. In one embodiment, the dose level is reduced for a second time.

In some embodiments, after the administration of Compound 1 or a pharmaceutically acceptable salt thereof, less than 40% of the patient population has the elevation of ALT or AST adverse event (e.g. TEAE or TRAE). In some embodiments, after the administration of Compound 1 or a pharmaceutically acceptable salt thereof, less than about 35%, less than about 30%, less than about 25%, less than about 20%, less than about 15%, less than about 10% or less than about 5% of the patient population has the elevation of ALT or AST adverse event (e.g. TEAE or TRAE). In some embodiments, less than about 15% of the patient population has the elevation of ALT or AST adverse event. In some embodiments, less than about 14% of the patient population has the elevation of ALT or AST adverse event. In some embodiments, less than about 13% of the patient population has the elevation of ALT or AST adverse event. In some embodiments, less than about 12% of the patient population has the elevation of ALT or AST adverse event. In some embodiments, less than about 11% of the patient population has the elevation of ALT or AST adverse event. In some embodiments, less than about 10% of the patient population has the elevation of ALT or AST adverse event. In some embodiments, less than about 9% of the patient population has the elevation of ALT or AST adverse event. In some embodiments, less than about 8% of the patient population has the elevation of ALT or AST adverse event. In some embodiments, less than about 7% of the patient population has the elevation of ALT or AST adverse event. In some embodiments, less than about 6% of the patient population has the elevation of ALT or AST adverse event. In some embodiments, less than about 5% of the patient population has the elevation of ALT or AST adverse event. In certain embodiments, the elevation of ALT or AST adverse event is Grade 1. In certain embodiments, the elevation of ALT or AST adverse event is Grade 2 (or lower). In certain embodiments, the elevation of ALT or AST adverse event is Grade 3 (or lower).

In one embodiment, the patient does not experience substantial hyperlipidemia after the administration of Compound 1 or a pharmaceutically acceptable salt. In one embodiment, the patient does not experience substantial increase of blood triglycerides after the administration of Compound 1 or a pharmaceutically acceptable salt. In one embodiment, the patient at most experiences at most Grade 3 increase of blood triglycerides as TEAE or TRAE after the administration of Compound 1 or a pharmaceutically acceptable salt. In one embodiment, the patient at most experiences Grade 2 increase of blood triglycerides as the TEAE or TRAE after the administration of Compound 1 or a pharmaceutically acceptable salt. In one embodiment, the patient at most experiences Grade 1 increase of blood triglycerides as the TEAE or TRAE after the administration of Compound 1 or a pharmaceutically acceptable salt. In one embodiment, the patient does not experience substantial increase of total cholesterol after the administration of Compound 1 or a pharmaceutically acceptable salt. In one embodiment, the patient at most experiences Grade 3 increase of total cholesterol as the TEAE or TRAE after the administration of Compound 1 or a pharmaceutically acceptable salt. In one embodiment, the patient at most experiences Grade 2 increase of total cholesterol as the TEAE or TRAE after the administration of Compound 1 or a pharmaceutically acceptable salt. In one embodiment, the patient at most experiences Grade 1 increase of total cholesterol as the TEAE or TRAE after the administration of Compound 1 or a pharmaceutically acceptable salt. In certain embodiments, the patient is further administered with a lipid-lowering agent. In some embodiments, after Compound 1 or a pharmaceutically acceptable salt thereof is administered at a dose level to a patient, the patient experiences Grade 1 increase of total cholesterol or triglyceride as TEAE or TRAE and the administration continues at the same dose level. In some embodiments, after Compound 1 or a pharmaceutically acceptable salt thereof is administered at a dose level to a patient, the patient experiences Grade 1 increase of total cholesterol or triglyceride as TEAE or TRAE and the administration continues at the same dose level with further administration of a lipid-lower agent. In some embodiments, after Compound 1 or a pharmaceutically acceptable salt thereof is administered at a dose level to a patient, the patient experiences Grade 2 increase of total cholesterol or triglyceride as TEAE or TRAE and the administration continues at the same dose level. In some embodiments, after Compound 1 or a pharmaceutically acceptable salt thereof is administered at a dose level to a patient, the patient experiences Grade 2 increase of total cholesterol or triglyceride as TEAE or TRAE and the administration continues at the same dose level with further administration of a lipid-lower agent. In some embodiments, after Compound 1 or a pharmaceutically acceptable salt thereof is administered at a dose level to a patient, the patient experiences Grade 3 increase of total cholesterol or triglyceride as TEAE or TRAE and the administration continues at the same dose level. In some embodiments, after Compound 1 or a pharmaceutically acceptable salt thereof is administered at a dose level to a patient, the patient experiences Grade 3 increase of total cholesterol or triglyceride as TEAE or TRAE and the administration is subject to dose interruption (i.e., the administration is stopped/withheld for a period of time) or modification (e.g. reduction). In some embodiments, the patient resumes the same dose level after the dose interruption or modification when the increase of total cholesterol or triglyceride returns to Grade 2 or lower, or the total cholesterol or triglyceride returns to baseline. In some embodiments, the patient experiences Grade 3 increase of total cholesterol or triglyceride as TEAE or TRAE and the administration continues at the same dose level with further administration of a lipid-lower agent. In some embodiments, the patient experiences Grade 3 increase of total cholesterol or triglyceride as TEAE or TRAE and the administration is subject to dose interruption or modification (e.g. reduction) with further administration of a lipid-lower agent. In some embodiments, the lipid-lower agent is selected from statins, fibrates, Niacin, Lecithin, bile acid sequestrants, ezetimibe, lomitapide, phytosterols, Omega-3, PCSK9 inhibitors, choline, L-arginine, flaxseed oil, and pycnogenol. In one embodiment, the dose reduction is done according to Table A.

In some embodiments, the administration of Compound 1 or a pharmaceutically acceptable salt is once daily. In some embodiments, the administration of Compound 1 or a pharmaceutically acceptable salt is twice daily. In some embodiments, the administration of Compound 1 or a pharmaceutically acceptable salt is once daily continuously for the dosing period. In some embodiments, the administration of Compound 1 or a pharmaceutically acceptable salt is twice daily continuously for the dosing period.

In one embodiment, the patient does not experience substantial increase of total bilirubin as the TEAE or TRAE after the administration of Compound 1. In one embodiment, the patient does not experience substantial increase of alkaline phosphatase after the administration of Compound 1.

As used herein in connection with an adverse event, and unless otherwise specified, “substantial” refers to about at least about 10% compared to the baseline. In some embodiments, “substantial” refers to about at least about 20% compared to the baseline. In some embodiments, “substantial” refers to about at least about 30% compared to the baseline. In some embodiments, “substantial” refers to about at least about 40% compared to the baseline. In some embodiments, “substantial” refers to about at least about 50% compared to the baseline. In some embodiments, “substantial” refers to about at least about 60% compared to the baseline. In some embodiments, “substantial” refers to about at least about 70% compared to the baseline. In some embodiments, “substantial” refers to about at least about 80% compared to the baseline. In some embodiments, “substantial” refers to about at least about 90% compared to the baseline. In some embodiments, the administration is once daily. In some embodiments, the administration is twice daily. In some embodiments, the administration is once daily continuously for the dosing period. In some embodiments, the administration is twice daily continuously for the dosing period.

In one embodiment, the patient does not experience a gastrointestinal disorder (e.g. constipation, diarrhoea, hypoesthesia oral, nausea, or vomiting) after the administration of Compound 1 or a pharmaceutically acceptable salt thereof. In one embodiment, the patient experiences at most Grade 3 gastrointestinal disorders (e.g. constipation, diarrhoea, hypoesthesia oral, nausea, or vomiting) as the treatment-emergent adverse event (TEAE) or treatment-related adverse event (TRAE). In one embodiment, the patient experiences at most a Grade 2 gastrointestinal disorder (e.g. constipation, diarrhoea, hypoesthesia oral, nausea, or vomiting) TEAE or TRAE. In one embodiment, the patient experiences at most a Grade 1 gastrointestinal disorder (e.g. constipation, diarrhoea, hypoesthesia oral, nausea, or vomiting) TEAE or TRAE. In certain embodiments, the patient does not experience substantial nausea, constipation, or diarrhoea. In certain embodiments, the patient at most experiences Grade 3 nausea, constipation, or diarrhoea as TEAE or TRAE. In certain embodiments, the patient at most experiences Grade 2 nausea, constipation, or diarrhoea as TEAE or TRAE. In certain embodiments, the patient at most experiences Grade 1 nausea, constipation, or diarrhoea as TEAE or TRAE. In some embodiments, after Compound 1 or a pharmaceutically acceptable salt thereof is administered at a dose level to a patient, the patient experiences Grade 1 a gastrointestinal disorder as TEAE or TRAE and the administration continues at the same dose level. In some embodiments, after Compound 1 or a pharmaceutically acceptable salt thereof is administered at a dose level to a patient, the patient experiences Grade 2 a gastrointestinal disorder as TEAE or TRAE and the administration continues at the same dose level. In some embodiments, after Compound 1 or a pharmaceutically acceptable salt thereof is administered at a dose level to a patient, the patient experiences Grade 2 a gastrointestinal disorder as TEAE or TRAE and the administration is subject to dose interruption (i.e., the administration is stopped/withheld for a period of time) or modification (e.g. reduction). In some embodiments, after Compound 1 or a pharmaceutically acceptable salt thereof is administered at a dose level to a patient, the patient experiences Grade 3 a gastrointestinal disorder as TEAE or TRAE and the administration is subject to dose interruption or modification (e.g. reduction). In some embodiments, the patient resumes the same dose level after the dose interruption or modification when the level of gastrointestinal disorder adverse event returns to Grade 2 or lower. In some embodiments, the patient resumes the same dose level after the dose interruption or modification when the level of gastrointestinal disorder adverse event returns to Grade 1 or lower. In certain embodiments, the patient is further administered with an antiemetic (e.g. bismuth subsalicylate, Antihistamine, Metoclopramide, etc.) to manage nausea. In certain embodiments, the patient is further administered with a laxative (e.g. oral osmotics like MiraLAX, oral stool softener like Colace, oral stimulants like Dulcolax, or rectal suppositories like Dulcolax) to manage constipation. In certain embodiments, the patient is further administered with an antidiarrheal agent (e.g. loperamide, bismuth subsalicylate, etc.) to manage diarrhea. In one embodiment, the dose reduction is done according to Table A.

In one embodiment, the patient experiences no musculoskeletal and connective tissue disorder (e.g. back pain, musculoskeletal chest pain, musculoskeletal pain, or pain in extremity) after administration of Compound 1 or a pharmaceutically acceptable salt thereof. In one embodiment, the patient experiences no musculoskeletal and connective tissue disorder (e.g. back pain, musculoskeletal chest pain, musculoskeletal pain, or pain in extremity) as the TEAE or TRAE after administration of Compound 1 or a pharmaceutically acceptable salt thereof. In one embodiment, the patient experiences at most Grade 3 musculoskeletal and connective tissue disorder (e.g. back pain, musculoskeletal chest pain, musculoskeletal pain, or pain in extremity) as the TEAE or TRAE after administration of Compound 1 or a pharmaceutically acceptable salt thereof. In one embodiment, the patient experiences at most Grade 2 musculoskeletal and connective tissue disorder (e.g. back pain, musculoskeletal chest pain, musculoskeletal pain, or pain in extremity) as the TEAE or TRAE after administration of Compound 1 or a pharmaceutically acceptable salt thereof. In one embodiment, the patient experiences at most Grade 1 musculoskeletal and connective tissue disorder (e.g. back pain, musculoskeletal chest pain, musculoskeletal pain, or pain in extremity) as the TEAE or TRAE after administration of Compound 1 or a pharmaceutically acceptable salt thereof. In certain embodiments, the patient experiences no substantial pain after administration of Compound 1 or a pharmaceutically acceptable salt thereof. In certain embodiments, the patient experiences Grade 1 level of pain as TEAE or TRAE after administration of Compound 1 or a pharmaceutically acceptable salt thereof. In certain embodiments, the patient experiences Grade 2 level of pain as TEAE or TRAE after administration of Compound 1 or a pharmaceutically acceptable salt thereof. In certain embodiments, the patient experiences Grade 3 level of pain as TEAE or TRAE after administration of Compound 1 or a pharmaceutically acceptable salt thereof. In some embodiments, the patient experiences Grade 2 level of a musculoskeletal and connective tissue disorder as TEAE or TRAE and is subject to dose interruption or modification (e.g. reduction). In some embodiments, after Compound 1 or a pharmaceutically acceptable salt thereof is administered at a dose level to a patient, the patient experiences Grade 3 level of a musculoskeletal and connective tissue disorder as TEAE or TRAE and the administration is subject to dose interruption (i.e., the administration is stopped/withheld for a period of time) or modification (e.g. reduction). In some embodiments, the patient resumes the same dose level after the dose interruption or modification when the level of musculoskeletal and connective tissue disorder adverse event returns to Grade 2 or lower. In some embodiments, the patient resumes the same dose level after the dose interruption or modification when the level of musculoskeletal and connective tissue disorder adverse event returns to Grade 1 or lower. In one embodiment, the patient is further administered with an analgesic (e.g. acetaminophen, NSAID,COX-2 inhibitor such as etoricoxib). In one embodiment, the dose reduction is done according to Table A.

In one embodiment, the patient experiences no respiratory, thoracic and mediastinal disorder (e.g. cough, dyspnoea, dyspnoea exertional, or increased bronchial secretion). In one embodiment, the patient experiences no respiratory, thoracic and mediastinal disorder (e.g. cough, dyspnoea, dyspnoea exertional, or increased bronchial secretion) as the TRAE or TEAE. In one embodiment, the patient experiences at most Grade 3 respiratory, thoracic and mediastinal disorder (e.g. cough, dyspnoea, dyspnoea exertional, or increased bronchial secretion) as the TRAE or TEAE. In one embodiment, the patient experiences at most Grade 2 respiratory, thoracic and mediastinal disorder (e.g. cough, dyspnoea, dyspnoea exertional, or increased bronchial secretion) as the TRAE or TEAE. In one embodiment, the patient experiences at most Grade 1 respiratory, thoracic and mediastinal disorder (e.g. cough, dyspnoea, dyspnoea exertional, or increased bronchial secretion) as the TRAE or TEAE. In one embodiment, the patient experiences at most Grade 1 respiratory, thoracic and mediastinal disorder (e.g. cough, dyspnoea, dyspnoea exertional, or increased bronchial secretion) as the TRAE or TEAE. In one embodiment, the patient experiences at most Grade 1 cough as the TRAE or TEAE. In one embodiment, the patient experiences at most Grade 2 cough as the TRAE or TEAE. In one embodiment, the patient experiences at most Grade 3 cough as the TRAE or TEAE. In one embodiment, the patient is further administered with a cough reliever (e.g. dextromethorphan, guaifenesin, menthol, antihistamines (e.g. chlorpheniramine and clemastine), decongestants (e.g. pseudoephedrine or phenylephrine), benzonatate, albuterol, promethazine, steroids (e.g. prednisone, prednisolone, dexamethasone, fluticasone propionate, budesonide, salbutamol plus ipratropium-bromide, montelukast, nociception-opioid-1-receptor agonist, codeine and gelatine, etc)). In one embodiment, the patient is further administered with prednisone (e.g. no more than 10 mg/day) as a cough reliever. In some embodiments, after Compound 1 or a pharmaceutically acceptable salt thereof is administered at a dose level to a patient, the patient experiences a Grade 1 respiratory, thoracic and mediastinal disorder (e.g. cough, dyspnoea, dyspnoea exertional, or increased bronchial secretion) and the administration continues at the same dose level. In some embodiments, after Compound 1 or a pharmaceutically acceptable salt thereof is administered at a dose level to a patient, the patient experiences a Grade 2 respiratory, thoracic and mediastinal disorder (e.g. cough, dyspnoea, dyspnoea exertional, or increased bronchial secretion) as TEAE or TRAE and the administration continues at the same dose level. In some embodiments, after Compound 1 or a pharmaceutically acceptable salt thereof is administered at a dose level to a patient, the patient experiences a Grade 2 respiratory, thoracic and mediastinal disorder (e.g. cough, dyspnoea, dyspnoea exertional, or increased bronchial secretion) as TEAE or TRAE and the administration is subject to dose interruption (i.e., the administration is stopped/withheld for a period of time) or modification (e.g. reduction). In some embodiments, after Compound 1 or a pharmaceutically acceptable salt thereof is administered at a dose level to a patient, the patient experiences a Grade 2 respiratory, thoracic and mediastinal disorder (e.g. cough, dyspnoea, dyspnoea exertional, or increased bronchial secretion) as TEAE or TRAE and the administration is subject to dose interruption until the adverse event severity is Grade 1 or lower. In some embodiments, after Compound 1 or a pharmaceutically acceptable salt thereof is administered at a dose level to a patient, the patient experiences a Grade 3 respiratory, thoracic and mediastinal disorder (e.g. cough, dyspnoea, dyspnoea exertional, or increased bronchial secretion) as TEAE or TRAE and the administration is subject to dose interruption or modification (e.g. reduction). In some embodiments, the patient resumes the same dose level after the dose interruption or modification when the respiratory, thoracic and mediastinal disorder adverse event returns to Grade 2 or lower. In some embodiments, the patient resumes the same dose level after the dose interruption or modification when the respiratory, thoracic and mediastinal disorder adverse event returns to Grade 1 or lower. In certain embodiments, the respiratory, thoracic and mediastinal disorder is cough. In one embodiment, the dose reduction is done according to Table A.

In one embodiment, the patient does not experience substantial weight gain after the administration of Compound 1 or a pharmaceutically acceptable salt. In one embodiment, the patient population does not experience statistically significant (e.g. ≥ about 7%, about 8%, about 9%, about 10%, about 11%, about 12%, about 13%, about 14%, about 15% of the baseline) weight gain after the administration of Compound 1 or a pharmaceutically acceptable salt. In one embodiment, the patient population does not experience statistically significant weight gain after the administration of Compound 1 or a pharmaceutically acceptable salt once daily for about one month. In one embodiment, the patient population does not experience statistically significant weight gain after the administration of Compound 1 or a pharmaceutically acceptable salt once daily for about two months. In one embodiment, the patient population does not experience statistically significant weight gain after the administration of Compound 1 or a pharmaceutically acceptable salt once daily for about three months. In one embodiment, the patient population does not experience statistically significant weight gain after the administration of Compound 1 or a pharmaceutically acceptable salt once daily for about four months. In one embodiment, the patient population does not experience statistically significant weight gain after the administration of Compound 1 or a pharmaceutically acceptable salt once daily for about five months. In one embodiment, the patient population does not experience statistically significant weight gain after the administration of Compound 1 or a pharmaceutically acceptable salt once daily for about six months. In one embodiment, the patient population does not experience statistically significant weight gain after the administration of Compound 1 or a pharmaceutically acceptable salt once daily for about 8 months. In one embodiment, the patient population does not experience statistically significant weight gain after the administration of Compound 1 or a pharmaceutically acceptable salt once daily for about 10 months. In one embodiment, the patient population does not experience statistically significant weight gain after the administration of Compound 1 or a pharmaceutically acceptable salt once daily for about 12 months. In some embodiments, the weigh gain is abdominal obesity. In some embodiments, the weigh gain is the increase in waist circumference. In some embodiments, the weight gain of the patient population is the mean increase in waist circumference. In some embodiments, the patient does not experience an obesity-related complication (e.g. diabetes mellitus (e.g. type 2), cardiovascular comorbidity, depression or osteoarthritis, thrombosis, increased risk of severe course of infection).

In one embodiment, the patient experiences no treatment-related serious adverse event (SAE). In one embodiment, the patient experiences no treatment-related CNS adverse event. In one embodiment, the patient experiences no treatment-related dizziness.

In one embodiment, the patient has a complete response after one or more cycles of treatment. In one embodiment, the patient has a partial response after one or more cycles of treatment. In one embodiment, the patient has reached stable disease after one or more cycles of treatment. In one embodiment, the patient has not experienced progressive disease after one or more cycles of treatment.

In one embodiment, the patient is a patient population. In one embodiment, the patient population does not experience statistically significant (e.g. ≥ about 7%, about 8%, about 9%, about 10%, about 11%, about 12%, about 13%, about 14%, about 15%, about 20%, about 25%, about 30%, about 35%, about 40% of the baseline) adverse event (e.g. TEAE or TRAE) as described herein. In one embodiment, the patient population does not experience statistically significant ≥ about 7% of the baseline adverse event (e.g. TEAE or TRAE) as described herein. In one embodiment, the patient population does not experience statistically significant ≥ about 8% of the baseline adverse event (e.g. TEAE or TRAE) as described herein. In one embodiment, the patient population does not experience statistically significant ≥ about 9% of the baseline adverse event (e.g. TEAE or TRAE) as described herein. In one embodiment, the patient population does not experience statistically significant ≥ about 10% of the baseline adverse event (e.g. TEAE or TRAE) as described herein. In one embodiment, the patient population does not experience statistically significant ≥ about 15% of the baseline adverse event (e.g. TEAE or TRAE) as described herein. In one embodiment, the patient population does not experience statistically significant ≥ about 20% of the baseline adverse event (e.g. TEAE or TRAE) as described herein. In one embodiment, the patient population does not experience statistically significant ≥ about 25% of the baseline adverse event (e.g. TEAE or TRAE) as described herein. In one embodiment, the patient population does not experience statistically significant ≥ about 30% of the baseline adverse event (e.g. TEAE or TRAE) as described herein. In one embodiment, the patient population does not experience statistically significant ≥ about 35% of the baseline adverse event (e.g. TEAE or TRAE) as described herein. In one embodiment, the patient population does not experience statistically significant ≥ about 40% of the baseline adverse event (e.g. TEAE or TRAE) as described herein. In one embodiment, the patient population does not experience statistically significant ≥ about 45% of the baseline adverse event (e.g. TEAE or TRAE) as described herein. In one embodiment, the adverse event is hepatotoxicity. In one embodiment, the adverse event is substantial elevation of transaminase. In one embodiment, the adverse event is substantial elevation of ALT or AST. In one embodiment, the adverse event is hyperlipidemia. In one embodiment, the adverse event is substantial increase of blood triglycerides. In one embodiment, the adverse event is substantial increase of total cholesterol. In one embodiment, the adverse event is substantial increase of total bilirubin. In one embodiment, the adverse event is a gastrointestinal disorder (e.g. constipation, diarrhoea, hypoesthesia oral, nausea, or vomiting). In one embodiment, the adverse event is a musculoskeletal and connective tissue disorder (e.g. back pain, musculoskeletal chest pain, musculoskeletal pain, or pain in extremity). In one embodiment, the adverse event is a respiratory, thoracic and mediastinal disorder (e.g. cough, dyspnoea, dyspnoea exertional, or increased bronchial secretion). In one embodiment, the adverse event is substantial weight gain. In one embodiment, the adverse event is TEAE. In one embodiment, the adverse event is TRAE. In one embodiment, the adverse event is Grade 1. In one embodiment, the adverse event is Grade 2. In one embodiment, the adverse event is Grade 3. In one embodiment, the adverse event is at most Grade 1. In one embodiment, the adverse event is at most Grade 2. In one embodiment, the adverse event is at most Grade 3.

As used herein and unless otherwise specified, Complete Response (CR) refers to the disappearance of all target lesion(s). In some embodiments of CR, any pathological lymph nodes (whether target or non-target) must have reduction in short axis to <10 mm. Partial Response (PR) refers to at least a 30% decrease in the sum of diameters of target lesion(s). Progressive Disease (PD) refers to at least a 20% increase in the sum of diameters of target lesion(s). In some embodiments of PD, in addition to the relative increase of 20%, the sum also demonstrates an increase of at least 5 mm. In some embodiments, the appearance of one or more new lesion(s) is also considered progression. Stable Disease (SD) refers to neither sufficient shrinkage to qualify for PR nor sufficient increase to qualify for PD, taking as reference the smallest sum diameters while on study. Additional description of these terms according to RECIST 1.1 can be found in European Journal of Cancer 45 (2009) 228-247.

In one embodiment, provided herein is a method of reducing a lesion in a subject having an ALK-positive solid tumor (e.g., NSCLC), comprising
(i) obtaining a first radiological measurement of the size of the lesion;
(ii) administering a pharmaceutically effective amount of Compound 1 once daily for one or more days; and
(iii) obtaining a second radiological measurement of the size of the lesion;
   wherein the size of the lesion determined from the second measurement is at most 100% of the size of the lesion determined from the first measurement. In some embodiments, the measurement of the size of the lesion is defined in RECIST 1.1 response criteria. In one embodiment, the lesion is a target lesion, e.g., as described in Example 1 or Example 15.

In one embodiment, the size of the lesion is characterized as the sum of diameters of the lesion. In one embodiment, the sum of diameters of the lesion determined from the second measurement is at most about 90% of the sum of diameters of the lesion determined from the first measurement. In one embodiment, the sum of diameters of the lesion determined from the second measurement is at most about 80% of the sum of diameters of the lesion determined from the first measurement. In one embodiment, the sum of diameters of the lesion determined from the second measurement is at most about 70% of the sum of diameters of the lesion determined from the first measurement. In one embodiment, the sum of diameters of the lesion determined from the second measurement is at most about 60% of the sum of diameters of the lesion determined from the first measurement. In one embodiment, the sum of diameters of the lesion determined from the second measurement is at most about 50% of the sum of diameters of the lesion determined from the first measurement. In one embodiment, the sum of diameters of the lesion determined from the second measurement is about 0.01% to about 90% of the sum of diameters of the lesion determined from the first measurement. In one embodiment, the second measurement shows no detectable lesion.

In one embodiment, the patient has brain metastases. In one embodiment, the patient has brain metastases and experiences no intracranial progression after at least one treatment cycle. In one embodiment, the patient has brain metastases and experiences no intracranial progression after at least two treatment cycles.

In one embodiment, the patient has at least about 5% to about 100% reduction of ALK variant allele(s) in circulating tumor DNA after at least one treatment cycle. In one embodiment, the patient has at least about 5% to about 100% reduction of ALK variant allele(s) in circulating tumor DNA after at least 15 days of treatment. In one embodiment, the patient has at least about 30% reduction of ALK variant allele(s) in circulating tumor DNA after at least one treatment cycle. In one embodiment, the patient has at least about 40% reduction of ALK variant allele(s) in circulating tumor DNA after at least one treatment cycle. In one embodiment, the patient has at least about 50% reduction of ALK variant allele(s) in circulating tumor DNA after at least one treatment cycle. In one embodiment, the patient has at least about 60% reduction of ALK variant allele(s) in circulating tumor DNA after at least one treatment cycle. In one embodiment, the patient has at least about 70% reduction of ALK variant allele(s) in circulating tumor DNA after at least one treatment cycle. In one embodiment, the patient has at least about 80% reduction of ALK variant allele(s) in circulating tumor DNA after at least one treatment cycle. In one embodiment, the patient has at least about 90% reduction of ALK variant allele(s) in circulating tumor DNA after at least one treatment cycle. In one embodiment, the patient has about 100% reduction of ALK variant allele(s) in circulating tumor DNA after at least one treatment cycle. In one embodiment, the patient has undetectable ALK variant allele(s) in circulating tumor DNA after at least one treatment cycle. In one embodiment, the patient has undetectable ALK variant allele(s) in circulating tumor DNA after at least one treatment cycles. In one embodiment, the ALK variant allele is one or more selected from G1202R, F1174L, F1174I, E1129K, S1206F, E1210K, G1269A, D1203N, and G1269V. In one embodiment, the ALK variant allele is G1202R. In one embodiment, the ALK variant allele is D1203N. In one embodiment, the ALK variant allele is F1174L and/or F1174I.

In one embodiment, the prior therapy is a prior ALK TKI therapy. In one embodiment, the ALK TKI is one or more selected from lorlatinib, crizotinib, ceritinib, alectinib, and brigatinib. In one embodiment, the prior therapies are an ALK TKI therapy and a systematic therapy. In one embodiment, the ALK TKI is one or more selected from lorlatinib, crizotinib, ceritinib, alectinib, brigatinib and the systematic therapy is one or more selected from pemetrexed, cisplatin, carboplatin, bevacizumab, datopotamab deruxtecan, trastuzumab deruxtecan (T-DXd, DS-8201a), and a checkpoint inhibitor (e.g. pembrolizumab, nivolumab, atezolizumab). In certain embodiments, the checkpoint inhibitor is a PD-1 inhibitor, a PD-L1 inhibitor, a cytotoxic T-lymphocyte-associated modulator (e.g., CTLA-4 inhibitors), an LAG-3 inhibitor, or a TIM-3 inhibitor. In certain embodiments, the PD-1 inhibitor or PD-L1 inhibitor is selected from pembrolizumab, nivolumab, atezolizumab, avelumab, durvalumab, cemiplimab, and dostarlimab. In certain embodiments, the PD-1 inhibitor is selected from pembrolizumab, nivolumab, atezolizumab, cemiplimab (LIBTAYO®), sintilimab, spartalizumab (PDR001), pidilizumab (CureTech), MEDI0680 (Medimmune), dostarlimab (TSR-042), PF-06801591 (Pfizer), sintilimab, toripalimab, tislelizumab (BGB-A317), camrelizumab (INCSHR1210, SHR-1210), AMP-224 (Amplimmune), CBT-501 (CBT Pharmaceuticals), CBT-502 (CBT Pharmaceuticals), JS001 (Junshi Biosciences), IBI308 (Innovent Biologics), INCSHR1210 (Incyte), also known as SHR-1210 (Hengrui Medicine), BGBA317 (Beigene), BGB-108 (Beigene), BAT-1306 (Bio-Thera Solutions), GLS-010 (Gloria Pharmaceuticals; WuXi Biologics), AK103, AK104, AK105 (Akesio Biopharma; Hangzhou Hansi Biologics; Hanzhong Biologics), LZM009 (Livzon), HLX-10 (Henlius Biotech), MEDI0680 (Medimmune), PDF001 (Novartis), PF-06801591 (Pfizer), pidilizumab (CureTech), REGN2810 (Regeneron) and TSR-042 (Tesaro), CS1003 (CStone Pharmaceuticals), or MEDI0680 (Medimmune). In certain embodiments, the PD-L1 inhibitor is selected from atezolizumab, FAZ053 (Novartis), and BMS-936559 (Bristol-Myers Squibb), KN035 (Alphamab; 3DMed; Ascletis Pharma), Envafolimab (TRACON Pharmaceuticals), BMS 936559 (Bristol-Myers Squibb), CS1001 (CStone Pharmaceuticals, Ligand Pharmaceuticals), CX-072 (CytomX Therapeutics), FAZ053 (Novartis), SHR-1316 (Hengrui Medicine), TQB2450 (Chiatai Tianqing), STI-A1014 (Zhaoke Pharm; Lee's Pharm, Lonza, Sorrento Therapeutics, NantWorks), LYN00102 (Lynkcell), A167 (Harbour BioMed, Kelun Group), BGB-A333 (Beigene), MSB2311 (Mabspace Biosciences), HLX-20 (Henlius Biotech), cosibelimab (Fortress Biotech), LY3300054 or lodapolimab (Eli Lilly), GS-4224 (Gilead Sciences), STI-A1015 (Yuhan, Sorrento Therapeutics), BCD-135 (BIOCAD), cosibelimab (Dana-Farber Cancer Institute, TG Therapeutics), APL-502 (Apollomics), AK106 (Akeso Biopharma), MSB2311 (Transcenta Holding), TG-1501 (TG Therapeutics) or FAZ053 (Novartis), MT-6035 (Molecular Templates), icaritin or ZKAB001 (Lonza, Lee's Pharmaceutical Holdings, Sorrento Therapeutics, Shenogen Pharma Group), TRIDENT Antibody (MacroGenics, Zai Lab), YBL-007 (Anh-Gook Pharmaceutical, Y-Biologics), HTI-1316 (Hengrui Therapeutics), PD-L1 Oncology Project (Weizmann Institute of Sciences), JS003 (Shanghai Junshi Biosciences), ND021 (Numab Therapeutics, CStone Pharmaceuticals), Toca 521 (Tocagen) or STT01 (STCube), DB004 (DotBio), MT-5050 (Molecular Templates), and KD036 (Kadmon).

In certain embodiments, the methods for treating or preventing cancer can be demonstrated by one or more responses such as increased apoptosis, inhibition of tumor growth, reduction of tumor metastasis, inhibition of tumor metastasis, reduction of microvessel density, decreased neovascularization, inhibition of tumor migration, tumor regression, and increased survival of the subject.

In one embodiment, administration of Compound 1 provides the area under the curve from 0 to 24 hours (AUC₀₋₂₄) of the compound in a range of (80% to 125% of 100 ng*h/mL) to (80% to 125% of 20,000 ng*h/mL). In one embodiment, administration of Compound 1 provides the area under the curve from 0 to 24 hours (AUC₀₋₂₄) of the compound in a range of about 100 to about 10,000 ng*h/mL. In one embodiment, the area under the curve from 0 to 24 hours (AUC₀₋₂₄) of the compound is in a range of about 200 to about 15,000 ng*h/mL after at least about 15 days of treatment (e.g. once daily for at least about 15 days). In one embodiment, the area under the curve from 0 to 24 hours (AUC₀₋₂₄) of the compound is in a range of about 300 to about 10,000 ng*h/mL after about 15 days of treatment (e.g. once daily for about 15 days). In one embodiment, the area under the curve from 0 to 24 hours (AUC₀₋₂₄) of the compound is in a range of about 1000 to about 9000 ng*h/mL after about 15 days of treatment (e.g. once daily for 15 days).

In one embodiment, such administration provides the area under the curve from 0 to 24 hours after administration (AUC₀₋₂₄) of the compound in a range of (80% to 125% of 10 ng*h/mL) to (80% to 125% of 300 ng*h/mL) for every mg of Compound 1 administered. In one embodiment, such administration provides the area under the curve from 0 to 24 hours after administration (AUC₀₋₂₄) of the compound in a range of from about 15 to about 200 ng*h/mL for every mg of Compound 1 administered. In one embodiment, such administration provides the area under the curve from 0 to 24 hours after administration (AUC₀₋₂₄) of the compound in a range of from about 20 to about 200 ng*h/mL after at least about 15 days of treatment (e.g. once daily for at least about 15 days). In one embodiment, such administration provides the area under the curve from 0 to 24 hours after administration (AUC₀₋₂₄) of the compound in a range of from about 20 to about 150 ng*h/mL after about 15 days of treatment (e.g. once daily for 15 days).

In one embodiment, such administration provides the maximum plasma concentration (Cₘₐₓ) of the compound in a range of (80% to 125% of 20 ng/mL) to (80% to 125% of 2000 ng/ml). In one embodiment, such administration provides the maximum plasma concentration (Cₘₐₓ) of the compound in a range of about 40 to about 1500 ng/ml. In one embodiment, such administration provides the maximum plasma concentration (Cₘₐₓ) of the compound in a range of about 40 to about 1000 ng/mL. In one embodiment, administration of Compound 1 provides the Cₘₐₓ of the compound in a range of about 50 to about 2000 ng*h/mL after about at least about 15 days of treatment (e.g. once daily for at least about 15 days). In one embodiment, administration of Compound 1 provides the Cₘₐₓ of the compound in a range of about 70 to about 1500 ng*h/mL after about 15 days of treatment (e.g. once daily for about 15 days). In one embodiment, administration of Compound 1 provides the Cₘₐₓ of the compound in a range of about 100 to about 1100 ng*h/mL after about 15 days of treatment (e.g. once daily for about 15 days).

In one embodiment, such administration provides the maximum plasma concentration (Cₘₐₓ) of the compound in a range of about 2 to about 70 ng/mL for every mg of Compound 1 administered. In one embodiment, such administration provides the maximum plasma concentration (Cₘₐₓ) of the compound in a range of about 2 to about 50 ng/ml for every mg of Compound 1 administered. In one embodiment, administration of Compound 1 provides the Cₘₐₓ of the compound in a range of about 3 to about 40 ng/ml for every mg of Compound 1 administered after at least about 15 days of treatment (e.g. once daily for at least about 15 days). In one embodiment, administration of Compound 1 provides the Cₘₐₓ of the compound in a range of about 3 to about 30 ng/mL for every mg of Compound 1 administered after about 15 days of treatment (e.g. once daily for about 15 days). In one embodiment, administration of Compound 1 provides the Cₘₐₓ of the compound in a range of about 3 to about 20 ng/ml for every mg of Compound 1 administered after about 15 days of treatment (e.g. once daily for about 15 days).

In one embodiment, such administration provides the Tₘₐₓ of the compound after the administration in a range of about 0.25 h to about 5 h. In one embodiment, such administration provides the Tₘₐₓ of the compound after the administration in a range of about 0.25 h to about 4 h. In one embodiment, such administration provides the Tₘₐₓ of the compound after the administration in a range of about 0.5 h to about 5 h after at least about 15 days of treatment (e.g. once daily for at least about 15 days). In one embodiment, such administration provides the Tₘₐₓ of the compound after the administration in a range of about 0.5 h to about 4 h after about 15 days of treatment (e.g. once daily for about 15 days). In one embodiment, such administration provides the Tₘₐₓ of the compound after the administration in a range of about 0.5 h to about 3.0 h after about 15 days of treatment (e.g. once daily for about 15 days).

In one embodiment, such administration provides the t_{1/2} of the compound after the administration in a range of about 3 h to about 100 h. In one embodiment, such administration provides the tin of the compound after the administration in a range of about 8 h to about 50 h. In one embodiment, such administration provides the t_{1/2} of the compound after the administration in a range of about 10 h to about 50 h after about 15 days of treatment.

In one embodiment, such administration provides the unbound plasma concentration after administration above about 1.4 ng/mL. In one embodiment, such administration provides the unbound plasma concentration after administration above about 4.2 ng/mL. In one embodiment, such administration provides the unbound plasma concentration after administration above about 12.5 ng/mL.

In one embodiment, the pharmacokinetic parameters provided herein (e.g., AUC₀₋₂₄, Cₘₐₓ, Tₘₐₓ, and half-life) refers to an arithmetic mean of a patient population. In one embodiment, the pharmacokinetic parameters provided herein (e.g., AUC₀₋₂₄, Cₘₐₓ) refers to a geometric mean of a patient population. In certain embodiments, t_{1/2} refers to the arithmetic mean of a patient population. In certain embodiments, Tₘₐₓ refers to the median range of a patient population. In certain embodiments, each of AUC₀₋₂₄, AUC_{last,unbound}, AUCₜₐᵤ, AUC_{tau_DN}, AUC_{inf}, AUC_{inf_DN}, Cₘₐₓ, C_{max_DN}, C_{max_unbound}, and Cₘᵢₙ refers to the geometric mean of a patient population.

In some embodiments, the subject or subject population has the QD administration of Compound 1 or a stereoisomer, or a mixture of stereoisomers thereof, or a pharmaceutically acceptable salt thereof. In some embodiments, the subject or subject population has the BID administration of Compound 1 or a stereoisomer, or a mixture of stereoisomers thereof, or a pharmaceutically acceptable salt thereof.

In certain embodiments, the measurement is after one administration of Compound 1 or a stereoisomer, or a mixture of stereoisomers thereof, or a pharmaceutically acceptable salt thereof. In certain embodiments, the measurement is after one cycle (e.g. 21 days) administration of Compound 1 or a stereoisomer, or a mixture of stereoisomers thereof, or a pharmaceutically acceptable salt thereof. In certain embodiments, the measurement is after about 15 days of administration of Compound 1 or a stereoisomer, or a mixture of stereoisomers thereof, or a pharmaceutically acceptable salt thereof. In certain embodiments, the measurement is after two cycles (e.g. 42 days) of administration of Compound 1 or a stereoisomer, or a mixture of stereoisomers thereof, or a pharmaceutically acceptable salt thereof. In certain embodiments, the measurement is after three cycles (e.g. 63 days) of administration of Compound 1 or a stereoisomer, or a mixture of stereoisomers thereof, or a pharmaceutically acceptable salt thereof. In certain embodiments, the measurement is after four cycles (e.g. 84 days) of administration of Compound 1 or a stereoisomer, or a mixture of stereoisomers thereof, or a pharmaceutically acceptable salt thereof.

### Combination Treatments

In some embodiments, the method of treating or preventing cancer may comprise administering Compound 1 conjointly with one or more other chemotherapeutic agent(s).

As used herein and unless otherwise specified, by “conjointly” or “in combination with”, it is not intended to imply that the other agent and Compound 1 must be administered at the same time and/or formulated for delivery together, although these methods of delivery are within the scope of this disclosure. The compound provided herein can be administered concurrently with, prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after), one or more other agents (e.g., one or more other additional agents). In general, each therapeutic agent is administered at a dose and/or on a time schedule determined for that particular agent. The other therapeutic agent can be administered with the compound provided herein in a single composition or separately in a different composition. Triple therapy is also contemplated herein.

Chemotherapeutic agents that may be conjointly administered with compounds of the disclosure include: 1-amino-4-phenylamino-9,10-dioxo-9,10-dihydroanthracene-2-sulfonate (acid blue 25), 1-amino-4-[4-hydroxyphenyl-amino]-9,10-dioxo-9,10-dihydroanthracene-2-sulfonate, 1-amino-4-[4-aminophenylamino]-9,10-dioxo-9,10-dihydroanthracene-2-sulfonate, 1-amino-4-[1-naphthylamino]-9,10-dioxo-9,10-dihydroanthracene-2-sulfonate, 1-amino-4-[4-fluoro-2-carboxyphenylamino]-9,10-dioxo-9,10-dihydroanthracene-2-sulfonate, 1-amino-4-[2-anthracenylamino]-9,10-dioxo-9,10-dihydroanthracene-2-sulfonate, ABT-263, afatinib dimaleate, axitinib, aminoglutethimide, amsacrine, anastrozole, APCP, asparaginase, AZD5363, Bacillus Calmette-Guérin vaccine (bcg), bicalutamide, bleomycin, bortezomib, β-methylene-ADP (AOPCP), buserelin, busulfan, cabazitaxel, cabozantinib, campothecin, capecitabine, carboplatin, carfilzomib, carmustine, ceritinib, chlorambucil, chloroquine, cisplatin, cladribine, clodronate, cobimetinib, colchicine, crizotinib, cyclophosphamide, cyproterone, cytarabine, dacarbazine, dactinomycin, daunorubicin, demethoxyviridin, dexamethasone, dichloroacetate, dienestrol, diethylstilbestrol, docetaxel, doxorubicin, epirubicin, eribulin, erlotinib, estradiol, estramustine, etoposide, everolimus, exemestane, filgrastim, fludarabine, fludrocortisone, fluorouracil, fluoxymesterone, flutamide, gefitinib, gemcitabine, genistein, goserelin, GSK1120212, hydroxyurea, idarubicin, ifosfamide, imatinib, interferon, irinotecan, ixabepilone, lenalidomide, letrozole, leucovorin, leuprolide, levamisole, lomustine, lonidamine, mechlorethamine, medroxyprogesterone, megestrol, melphalan, mercaptopurine, mesna, metformin, methotrexate, miltefosine, mitomycin, mitotane, mitoxantrone, MK-2206, mutamycin, N-(4-sulfamoylphenylcarbamothioyl) pivalamide, NF279, NF449, nilutamide, nocodazole, octreotide, olaparib, oxaliplatin, paclitaxel, pamidronate, pazopanib, pemexetred, pentostatin, perifosine, PF-04691502, plicamycin, pomalidomide, porfimer, PPADS, procarbazine, quercetin, raltitrexed, ramucirumab, reactive blue 2, rituximab, rolofylline, romidepsin, rucaparib, selumetinib, sirolimus, sodium 2,4-dinitrobenzenesulfonate, sorafenib, streptozocin, sunitinib, suramin, talazoparib, tamoxifen, temozolomide, temsirolimus, teniposide, testosterone, thalidomide, thioguanine, thiotepa, titanocene dichloride, tonapofylline, topotecan, trametinib, trastuzumab, tretinoin, veliparib, vinblastine, vincristine, vindesine, vinorelbine, and vorinostat (SAHA). In other embodiments, chemotherapeutic agents that may be conjointly administered with compounds of the disclosure include: ABT-263, dexamethasone, 5-fluorouracil, PF-04691502, romidepsin, and vorinostat (SAHA). In other embodiments, chemotherapeutic agents that may be conjointly administered with compounds of the disclosure include: 1-amino-4-phenylamino-9,10-dioxo-9,10-dihydroanthracene-2-sulfonate (acid blue 25), 1-amino-4-[4-hydroxyphenyl-amino]-9,10-dioxo-9,10-dihydroanthracene-2-sulfonate, 1-amino-4-[4-aminophenylamino]-9,10-dioxo-9,10-dihydroanthracene-2-sulfonate, 1-amino-4-[1-naphthylamino]-9,10-dioxo-9,10-dihydroanthracene-2-sulfonate, 1-amino-4-[4-fluoro-2-carboxyphenylamino]-9,10-dioxo-9,10-dihydroanthracene-2-sulfonate, 1-amino-4-[2-anthracenylamino]-9,10-dioxo-9,10-dihydroanthracene-2-sulfonate, APCP, β-methylene-ADP (AOPCP), capecitabine, cladribine, cytarabine, fludarabine, doxorubicin, gemcitabine, N-(4-sulfamoylphenylcarbamothioyl) pivalamide, NF279, NF449, PPADS, quercetin, reactive blue 2, rolofylline sodium 2,4-dinitrobenzenesulfonate, suramin, and tonapofylline.

Many combination therapies have been developed for the treatment of cancer. In certain embodiments, a compound or pharmaceutical composition provided herein (e.g., Compound 1) may be conjointly administered with one or more combination therapies. Examples of combination therapies with which compounds provided herein may be conjointly administered are included in Table 1.

TABLE 1
Exemplary combinatorial therapies for the treatment of cancer

| Name | Therapeutic agents |
|---|---|
| ABV | Doxorubicin, Bleomycin, Vinblastine |
| ABVD | Doxorubicin, Bleomycin, Vinblastine, Dacarbazine |
| AC (Breast) | Doxorubicin, Cyclophosphamide |
| AC (Sarcoma) | Doxorubicin, Cisplatin |
| AC (Neuroblastoma) | Cyclophosphamide, Doxorubicin |
| ACE | Cyclophosphamide, Doxorubicin, Etoposide |
| ACe | Cyclophosphamide, Doxorubicin |
| AD | Doxorubicin, Dacarbazine |
| AP | Doxorubicin, Cisplatin |
| ARAC-DNR | Cytarabine, Daunorubicin |
| B-CAVe | Bleomycin, Lomustine, Doxorubicin, Vinblastine |
| BCVPP | Carmustine, Cyclophosphamide, Vinblastine, Procarbazine, |
| | Prednisone |
| BEACOPP | Bleomycin, Etoposide, Doxorubicin, Cyclophosphamide, |
| | Vincristine, Procarbazine, Prednisone, Filgrastim |
| BEP | Bleomycin, Etoposide, Cisplatin |
| BIP | Bleomycin, Cisplatin, Ifosfamide, Mesna |
| BOMP | Bleomycin, Vincristine, Cisplatin, Mitomycin |
| CA | Cytarabine, Asparaginase |
| CABO | Cisplatin, Methotrexate, Bleomycin, Vincristine |
| CAF | Cyclophosphamide, Doxorubicin, Fluorouracil |
| CAL-G | Cyclophosphamide, Daunorubicin, Vincristine, Prednisone, |
| | Asparaginase |
| CAMP | Cyclophosphamide, Doxorubicin, Methotrexate, |
| | Procarbazine |
| CAP | Cyclophosphamide, Doxorubicin, Cisplatin |
| CAV | Cyclophosphamide, Doxorubicin, Vincristine |
| CAVE ADD | CAV and Etoposide |
| CA-VP16 | Cyclophosphamide, Doxorubicin, Etoposide |
| CC | Cyclophosphamide, Carboplatin |
| CDDP/VP-16 | Cisplatin, Etoposide |
| CEF | Cyclophosphamide, Epirubicin, Fluorouracil |
| CEPP(B) | Cyclophosphamide, Etoposide, Prednisone, with or without/ |
| | Bleomycin |
| CEV | Cyclophosphamide, Etoposide, Vincristine |
| CF | Cisplatin, Fluorouracil or Carboplatin Fluorouracil |
| CHAP | Cyclophosphamide or Cyclophosphamide, Altretamine, |
| | Doxorubicin, Cisplatin |
| ChlVPP | Chlorambucil, Vinblastine, Procarbazine, Prednisone |
| CHOP | Cyclophosphamide, Doxorubicin, Vincristine, Prednisone |
| CHOP-BLEO | Add Bleomycin to CHOP |
| CISCA | Cyclophosphamide, Doxorubicin, Cisplatin |
| CLD-BOMP | Bleomycin, Cisplatin, Vincristine, Mitomycin |
| CMF | Methotrexate, Fluorouracil, Cyclophosphamide |
| CMFP | Cyclophosphamide, Methotrexate, Fluorouracil, Prednisone |
| CMFVP | Cyclophosphamide, Methotrexate, Fluorouracil, |
| | Vincristine, Prednisone |
| CMV | Cisplatin, Methotrexate, Vinblastine |
| CNF | Cyclophosphamide, Mitoxantrone, Fluorouracil |
| CNOP | Cyclophosphamide, Mitoxantrone, Vincristine, Prednisone |
| COB | Cisplatin, Vincristine, Bleomycin |
| CODE | Cisplatin, Vincristine, Doxorubicin, Etoposide |
| COMLA | Cyclophosphamide, Vincristine, Methotrexate, Leucovorin, |
| | Cytarabine |
| COMP | Cyclophosphamide, Vincristine, Methotrexate, Prednisone |
| Cooper Regimen | Cyclophosphamide, Methotrexate, Fluorouracil, |
| | Vincristine, Prednisone |
| COP | Cyclophosphamide, Vincristine, Prednisone |
| COPE | Cyclophosphamide, Vincristine, Cisplatin, Etoposide |
| COPP | Cyclophosphamide, Vincristine, Procarbazine, Prednisone |
| CP(Chronic | Chlorambucil, Prednisone |
| lymphocytic leukemia) | |
| CP (Ovarian Cancer) | Cyclophosphamide, Cisplatin |
| CVD | Cisplatin, Vinblastine, Dacarbazine |
| CVI | Carboplatin, Etoposide, Ifosfamide, Mesna |
| CVP | Cyclophosphamide, Vincristine, Prednisome |
| CVPP | Lomustine, Procarbazine, Prednisone |
| CYVADIC | Cyclophosphamide, Vincristine, Doxorubicin, Dacarbazine |
| DA | Daunorubicin, Cytarabine |
| DAT | Daunorubicin, Cytarabine, Thioguanine |
| DAV | Daunorubicin, Cytarabine, Etoposide |
| DCT | Daunorubicin, Cytarabine, Thioguanine |
| DHAP | Cisplatin, Cytarabine, Dexamethasone |
| DI | Doxorubicin, Ifosfamide |
| DTIC/Tamoxifen | Dacarbazine, Tamoxifen |
| DVP | Daunorubicin, Vincristine, Prednisone |
| EAP | Etoposide, Doxorubicin, Cisplatin |
| EC | Etoposide, Carboplatin |
| EFP | Etoposie, Fluorouracil, Cisplatin |
| ELF | Etoposide, Leucovorin, Fluorouracil |
| EMA 86 | Mitoxantrone, Etoposide, Cytarabine |
| EP | Etoposide, Cisplatin |
| EVA | Etoposide, Vinblastine |
| FAC | Fluorouracil, Doxorubicin, Cyclophosphamide |
| FAM | Fluorouracil, Doxorubicin, Mitomycin |
| FAMTX | Methotrexate, Leucovorin, Doxorubicin |
| FAP | Fluorouracil, Doxorubicin, Cisplatin |
| F-CL | Fluorouracil, Leucovorin |
| FEC | Fluorouracil, Cyclophosphamide, Epirubicin |
| FED | Fluorouracil, Etoposide, Cisplatin |
| FL | Flutamide, Leuprolide |
| FZ | Flutamide, Goserelin acetate implant |
| HDMTX | Methotrexate, Leucovorin |
| Hexa-CAF | Altretamine, Cyclophosphamide, Methotrexate, |
| | Fluorouracil |
| IDMTX/6-MP | Methotrexate, Mercaptopurine, Leucovorin |
| IE | Ifosfamide, Etoposie, Mesna |
| IfoVP | Ifosfamide, Etoposide, Mesna |
| IPA | Ifosfamide, Cisplatin, Doxorubicin |
| M-2 | Vincristine, Carmustine, Cyclophosphamide, |
| | Prednisone, Melphalan |
| MAC-III | Methotrexate, Leucovorin, Dactinomycin, |
| | Cyclophosphamide |
| MACC | Methotrexate, Doxorubicin, Cyclophosphamide, |
| | Lomustine |
| MACOP-B | Methotrexate, Leucovorin, Doxorubicin, |
| | Cyclophosphamide, Vincristine, Bleomycin, Prednisone |
| MAID | Mesna, Doxorubicin, Ifosfamide, Dacarbazine |
| m-BACOD | Bleomycin, Doxorubicin, Cyclophosphamide, Vincristine, |
| | Dexamethasone, Methotrexate, Leucovorin |
| MBC | Methotrexate, Bleomycin, Cisplatin |
| MC | Mitoxantrone, Cytarabine |
| MF | Methotrexate, Fluorouracil, Leucovorin |
| MICE | Ifosfamide, Carboplatin, Etoposide, Mesna |
| MINE | Mesna, Ifosfamide, Mitoxantrone, Etoposide |
| mini-BEAM | Carmustine, Etoposide, Cytarabine, Melphalan |
| MOBP | Bleomycin, Vincristine, Cisplatin, Mitomycin |
| MOP | Mechlorethamine, Vincristine, Procarbazine |
| MOPP | Mechlorethamine, Vincristine, Procarbazine, Prednisone |
| MOPP/ABV | Mechlorethamine, Vincristine, Procarbazine, Prednisone, |
| | Doxorubicin, Bleomycin, Vinblastine |
| MP (multiple myeloma) | Melphalan, Prednisone |
| MP (prostate cancer) | Mitoxantrone, Prednisone |
| MTX/6-MO | Methotrexate, Mercaptopurine |
| MTX/6-MP/VP | Methotrexate, Mercaptopurine, Vincristine, Prednisone |
| MTX-CDDPAdr | Methotrexate, Leucovorin, Cisplatin, Doxorubicin |
| MV (breast cancer) | Mitomycin, Vinblastine |
| MV (acute myelocytic | Mitoxantrone, Etoposide |
| leukemia) | |
| M-VAC Methotrexate | Vinblastine, Doxorubicin, Cisplatin |
| MVP Mitomycin | Vinblastine, Cisplatin |
| MVPP | Mechlorethamine, Vinblastine, Procarbazine, Prednisone |
| NFL | Mitoxantrone, Fluorouracil, Leucovorin |
| NOVP | Mitoxantrone, Vinblastine, Vincristine |
| OPA | Vincristine, Prednisone, Doxorubicin |
| OPPA | Add Procarbazine to OPA. |
| PAC | Cisplatin, Doxorubicin |
| PAC-I | Cisplatin, Doxorubicin, Cyclophosphamide |
| PA-CI | Cisplatin, Doxorubicin |
| PCV | Lomustine, Procarbazine, Vincristine |
| PFL | Cisplatin, Fluorouracil, Leucovorin |
| POC | Prednisone, Vincristine, Lomustine |
| ProMACE | Prednisone, Methotrexate, Leucovorin, Doxorubicin, |
| | Cyclophosphamide, Etoposide |
| ProMACE/cytaBOM | Prednisone, Doxorubicin, Cyclophosphamide, Etoposide, |
| | Cytarabine, Bleomycin, Vincristine, Methotrexate, |
| | Leucovorin, Cotrimoxazole |
| proMACE/MOPP | Prednisone, Doxorubicin, Cyclophosphamide, Etoposide, |
| | Mechlorethamine, Vincristine, Procarbazine, Methotrexate, |
| | Leucovorin |
| Pt/VM | Cisplatin, Teniposide |
| PVA | Prednisone, Vincristine, Asparaginase |
| PVB | Cisplatin, Vinblastine, Bleomycin |
| PVDA | Prednisone, Vincristine, Daunorubicin, Asparaginase |
| SMF | Streptozocin, Mitomycin, Fluorouracil |
| TAD | Mechlorethamine, Doxorubicin, Vinblastine, Vincristine, |
| | Bleomycin, Etoposide, Prednisone |
| TTT | Methotrexate, Cytarabine, Hydrocortisone |
| Topo/CTX | Cyclophosphamide, Topotecan, Mesna |
| VAB-6 | Cyclophosphamide, Dactinomycin, Vinblastine, Cisplatin, |
| | Bleomycin |
| VAC | Vincristine, Dactinomycin, Cyclophosphamide |
| VACAdr | Vincristine, Cyclophosphamide, Doxorubicin, |
| | Dactinomycin, Vincristine |
| VAD | Vincristine, Doxorubicin, Dexamethasone |
| VATH | Vinblastine, Doxorubicin, Thiotepa, Flouxymesterone |
| VBAP | Vincristine, Carmustine, Doxorubicin, Prednisone |
| VBCMP | Vincristine, Carmustine, Melphalan, Cyclophosphamide, |
| | Prednisone |
| VC | Vinorelbine, Cisplatin |
| VCAP | Vincristine, Cyclophosphamide, Doxorubicin, Prednisone |
| VD | Vinorelbine, Doxorubicin |
| VelP | Vinblastine, Cisplatin, Ifosfamide, Mesna |
| VIP | Etoposide, Cisplatin, Ifosfamide, Mesna |
| VM | Mitomycin, Vinblastine |
| VMCP | Vincristine, Melphalan, Cyclophosphamide, Prednisone |
| VP | Etoposide, Cisplatin |
| V-TAD | Etoposide, Thioguanine, Daunorubicin, Cytarabine |
| 5 + 2 | Cytarabine, Daunorubicin, Mitoxantrone |
| 7 + 3 | Cytarabine with/, Daunorubicin or Idarubicin or |
| | Mitoxantrone |
| “8 in 1” | Methylprednisolone, Vincristine, Lomustine, Procarbazine, |
| | Hydroxyurea, Cisplatin, Cytarabine, Dacarbazine |

In certain embodiments, the conjoint therapies of the disclosure comprise conjoint administration with other types of therapeutic agents, such as immuno-oncology agents. Cancer cells often have specific cell surface antigens that can be recognized by the immune system. Thus, immuno-oncology agents, such as monoclonal antibodies, can selectively bind to tumor antigens and effect cell death. Other immuno-oncology agents can suppress tumor-mediated inhibition of the native immune response or otherwise activate the immune response and thus facilitate recognition of the tumor by the immune system. Exemplary antibody immuno-oncology agents, include, but are not limited to, abagovomab, adecatumumab, afutuzumab, alemtuzumab, anatumomab mafenatox, apolizumab, blinatumomab, BMS-936559, catumaxomab, durvalumab, epacadostat, epratuzumab, indoximod, inotuzumab ozogamicin, intetumumab, ipilimumab, isatuximab, lambrolizumab, MED14736, MPDL3280A, nivolumab, obinutuzumab, ocaratuzumab, ofatumumab, olaratumab, pembrolizumab, pidilizumab, rituximab, ticilimumab, samalizumab, and tremelimumab. In some embodiments, the antibody immuno-oncology agents are selected from anti-CD73 monoclonal antibody (mAb), anti-CD39 mAb, anti-PD-1 mAb, anti-PD-L1 mAb, and anti-CTLA4 mAb. Thus, in some embodiments, the methods of the disclosure comprise conjoint administration of one or more immuno-oncology agents, such as the agents mentioned above.

In some embodiments, the combination therapy comprises conjoint administration of a compound of the disclosure, such as Compound 1 (e.g., Form 2 of Compound 1), with SH2 inhibitors, such as CGP78850, CPG85793, C90, C126, G7-18NATE, G7-B1, and NSC642056.

Acquired ALK fusion confers EGFR inhibitor resistance. See von Buttlar, X. et al JTO Clin Res Rep. 2021 June; 2(6):100179; Schrock, A. B. et al J Thorac Oncol. 2018 September; 13(9):1312-1323. In some embodiments, the combination therapy comprises conjoint administration of a compound of the disclosure, such as Compound 1 (e.g., Form 2 of Compound 1), with an EGFR inhibitor, such as erlotinib, osimertinib, cetuximab, gefitinib, necitumumab, lapatinib, neratinib, panitumumab, vandetanib, and necitumumab. In one embodiment, the EGFR inhibitor is erlotinib. In one embodiment, the EGFR inhibitor is osimertinib. In one embodiment, the EGFR inhibitor is gefitinib.

In some embodiments, provided herein is a method of treating an epidermal growth factor receptor (EGFR)-associated cancer, comprising administering to a subject in need thereof a combination comprising a therapeutically effective amount of Compound 1 (e.g., Form 2 of Compound 1), or a pharmaceutically acceptable salt, or stereoisomer thereof, and therapeutically effective amount of a second agent, wherein the second agent is an EGFR inhibitor. In some embodiments, the EGFR inhibitor is selected from the group consisting of erlotinib, osimertinib, cetuximab, gefitinib, necitumumab, lapatinib, neratinib, panitumumab, vandetanib, and necitumumab. In one embodiment, the EGFR inhibitor is erlotinib. In one embodiment, the EGFR inhibitor is osimertinib. In one embodiment, the EGFR inhibitor is gefitinib. In some embodiments, the subject has at least one EGFR mutation. In some embodiments, the at least one EGFR mutations is in one or more exons of 18, 19, 20, and 21 and/or TM. In some embodiments, the subject has at least one receptor tyrosine kinase (RTK) mutation. In some embodiments, the RTK mutation is an ALK mutation. In some embodiments, the EGFR-associated cancer is NSCLC, pancreatic cancer, breast cancer, or colon cancer. In some embodiments, the EGFR-associated cancer is mediated by EGFR.

In some embodiments, the combination therapy comprises conjoint administration of a compound of the disclosure, such as Compound 1 (e.g., Form 2 of Compound 1), with a Raf (e.g. A-Raf, B-Raf, or C-Raf also known as Raf-1) inhibitor. In certain embodiments, the Raf inhibitor inhibits a Raf mutant. In certain embodiments, the Raf inhibitor is a B-Raf inhibitor. In certain embodiments, the B-Raf inhibitor inhibits a B-Raf mutant. In certain embodiments, the B-Raf mutant is a V600 mutant. In certain embodiments, the B-Raf mutant is a V600E mutant. In certain embodiments, the Raf inhibitor is dabrafenib, Y3009120, MLN2480/TAK-580 (tovorafenib), CCT196969, CCT241161, BGB659, PLX7904, naporafenib, PLX-8394, or lifirafenib.

In some embodiments, the combination therapy comprises conjoint administration of a compound of the disclosure, such as Compound 1 (e.g., Form 2 of Compound 1), with a PIK3CA inhibitor, such as 5-(2,6-di-morpholin-4-yl-pyrimidin-4-yl)-4-trifluoromethyl-pyridin-2-ylamine, inavolisib, gedatolisib, buparlisib, zandelisib, miransertib mesylate, izorlisib, or panulisib. In some embodiments, the PIK3CA inhibitor is a PI3K inhibitor, such as dactolisib, apitolisib, gedatolisib, SF1126, omipalisib, samotolisib, bimiralisib, paxalisib, voxtalisib buparlisib, CH5132799, pilaralisib, ZSTK474, sonolisib, pictilisib, copanlisib, B591, TG-100-115, RIDR-PI-103 alpelisib (α) or serabelisib (α).

ALK tyrosine kinase inhibitors are used in the treatment of cancer (e.g. a solid tumor) that are driven by constitutive ALK activity. In some embodiments, the combination therapy comprises conjoint administration of a compound of the disclosure, such as Compound 1 (e.g., Form 2 of Compound 1), with a CDK4 inhibitor or a CDK6 inhibitor, such as ribociclib, palboclib, trilaciclib, abemaciclib, or dalpiciclib.

Signal transducer and activator of transcription 3 (STAT3) is activated in many cancers, including those with constitutively active tyrosine kinases such as ALK. See Taniguchi, K et al Sci. Rep. 2021 Mar. 23; 11(1):6685, which is incorporated herein by reference in its entirety. In some embodiments, the combination therapy comprises conjoint administration of a compound of the disclosure, such as Compound 1 (e.g., Form 2 of Compound 1), with a STAT3 inhibitor, such as HL-237, OPB-111077, danvatirsen, C-188-9, KT-333, DUET-01, ENMD-1198, or WP-1066.

T-LAK cell-oriented protein kinase (TOPK, also referred to as PDZ binding kinase, gene symbol PBK) was identified as a substrate of ALK. Treatment of ALK fusion-driven human cancer cells with an ALK inhibitor and a TOPK inhibitor has shown anti-tumor activity. See Xiao, J. et al Cell Death Dis. 2022 Sep. 27; 13 (9): 828, which is incorporated herein by reference in its entirety. In some embodiments, the combination therapy comprises conjoint administration of a compound of the disclosure, such as Compound 1 (e.g., Form 2 of Compound 1), with a STAT3 inhibitor, such as SKLB-C05, ADA-07, OTS-964, or HI-032 (HITOPK-032).

In ALK fusion-driven NSCLC, EML4 is the major fusion partner. Wild-type EML4 is a microtubule-associated protein that is reported to promote microtubule stabilization in interphase. More than 10 distinct EML4-ALK variants have been identified, with variants 1 and 3 (V1 and V3) being the most prevalent. EML4-ALK V3 localizes to interphase microtubules and the mitotic spindle. Accordingly, treatment of EML3-ALK V3-expressing cells with a combination of ALK inhibitor and a microtubule modulator (e.g. stabilizer or destabilizer) results in synergistic antiproliferative activity in vitro. See Lucken, K. et al Mol. Cancer Res. 2022 Jun. 3; 20(6):854, which is incorporated herein by reference in its entirety. In some embodiments, the combination therapy comprises conjoint administration of a compound of the disclosure, such as Compound 1 (e.g., Form 2 of Compound 1), with taxanes (e.g. paclitaxel, nab-paclitaxel, docetaxel, and cabazitaxel), epothilones (e.g. epothilone A epothilone D, ixabepilone, and sagopilone) tirbanibulin, eribulin mesilate, vinflunine, vinorelbine, narcosine, vincristine sulfate, or vinblastine sulfatec colchicine.

HER2-directed therapies in combination with ALK inhibitor therapy may be beneficial for patients with HER2 amplification and ALK fusions. See Tsui, D. C. C. et al Clin. Lung Cancer 2022 March; 23(2):e99. In some embodiments, the combination therapy comprises conjoint administration of a compound of the disclosure, such as Compound 1 (e.g., Form 2 of Compound 1), with a HER2-directed therapy, such as trastuzumab, pertuzumab, margetuximab, ado-trastuzumab emtansine (T-DM1), trastuzumab deruxtecan (fam-trastuzumab deruxtecan), tucatinib, neratinib, lapatinib, disitamab vedotin, mobocertinib, trastuzumab hyaluronidase, pyrotinib maleate, dacomitinib, or afatinib.

Combinations of ALK TKI and anti-angiogenic agents may provide patient benefit, for example delaying progression of ALK fusion-positive cancers such as NSCLC. See Lin, J. J. et al ESMO Open 2022 February; 7(1): 100342. In some embodiments, the combination therapy comprises conjoint administration of a compound of the disclosure, such as Compound 1 (e.g., Form 2 of Compound 1), with an anti-angiogenic agent, such as axitinib, bevacizumab, cabozantinib, everolimus, lenalidomide (Revlimid®), lenvatinib mesylate, pazopanib, ramucirumab, regorafenib, sorafenib, sunitinib, thalidomide, vandetanib, ziv-aflibercept, sulfatinib, aplidine, anlotinib hydrochloride, fruquintinib, tivozanib, conbercept, apatinib mesylate, ponatinib, cabozantinib S-malate, aflibercept, sunitinib malate, or anecortave acetate.

In vitro, the JNK pathway was determined to be active upon extended high-dose treatment of ALK fusion-driven cells with ALK tyrosine kinase inhibitors (TKI), and may enable resistance to ALK TKIs to emerge (Cancer Lett, 2021 Dec. 1; 522:119-128). Accordingly, JNK inhibition may sensitize ALK fusion-driven cells to ALK TKIs in vitro and provide increased durability of antitumor activity. In some embodiments, the combination therapy comprises conjoint administration of a compound of the disclosure, such as Compound 1 (e.g., Form 2 of Compound 1), with a JNK inhibitor, such as SP600125, BMS-986360, or JNK-401.

Patients with ALK fusion-positive and TP53-mutated NSCLC may have a worse progression-free survival compared to those patients with ALK fusion-positive and wild-type TP53 NSCLC. In preclinical studies, p53-mutated ALK fusion-positive cells were resistant to apoptosis induction by ALK tyrosine kinase inhibition, but combination treatment with a proteosome inhibitor appeared to induce apoptosis and provide increased antitumor activity in ALK/TP53 co-mutated NSCLC tumors compared to single agent activity. See Tanimoto, A. et al Clin. Cancer Res. 2021 Mar. 1; 27(5):1410. In some embodiments, the combination therapy comprises conjoint administration of a compound of the disclosure, such as Compound 1 (e.g., Form 2 of Compound 1), with a proteosome inhibitor, such as ixazomib, carfilzomib, or bortezomib.

In ALK fusion-driven cells, ALK signaling may include activation of the mammalian target of rapamycin (mTOR). The combination of an ALK inhibitor and mTOR inhibitor may induce increased cell cycle arrest in ALK fusion-positive anaplastic large cell lymphoma (ALCL) cells. See Kim, D. et al Anticancer Res. 2020 March; 40(3):1395. In some embodiments, the combination therapy comprises conjoint administration of a compound of the disclosure, such as Compound 1 (e.g., Form 2 of Compound 1), with a mTOR inhibitor, such as everolimus, ABI-009, temsirolimus, zotarolimus, rapamycin, or umirolimus.

The transcriptional regulator YAP1 may be activated following treatment of ALK+ cells with an ALK inhibitor. This effect can be attenuated by combinatorial inhibition of ALK and YAP1. See Tsuji, T. et al Nat. Commun. 2020; 11:74. In some embodiments, the combination therapy comprises conjoint administration of a compound of the disclosure, such as Compound 1 (e.g., Form 2 of Compound 1), with a YAP1 inhibitor, such as narciclasine, MYF-03-69, verteporfin, IAG-933, ION-537, IK-930, or VT-3989.

In some embodiments, the combination therapy comprises conjoint administration of a compound of the disclosure, such as Compound 1 (e.g., Form 2 of Compound 1), with MEK inhibitors, such as trametinib, cobimetinib, binimetinib, selumetinib, PD-325901, CI-1040, and TAK-733. See “A Phase IB/II Study of Alectinib Combined With Cobimetinib in Advanced ALK-Rearranged (ALK+) NSCLC”, Clinical Trial Identifier: NCT03202940, Last Updated on Mar. 9, 2021, which is incorporated herein by reference in its entirety.

In some embodiments, the combination therapy comprises conjoint administration of a compound of the disclosure, such as Compound 1 (e.g., Form 2 of Compound 1), with a MET inhibitor selected from JNJ-38877605, PF-04217903, foretinib, AMG 458, tivantinib, cabozantinib, crizotinib, capmatinib hydrochloride, tepotinib hydrochloride, and savolitinib. See Tanizaki J. et al Br J Cancer, 2012 Feb. 14; 106(4):763-7, which is incorporated herein by reference in its entirety.

In some embodiments, the combination therapy comprises conjoint administration of a compound of the disclosure, such as Compound 1 (e.g., Form 2 of Compound 1), with a SHP2 inhibitor, such as TNO-155, RMC-4630, JAB-3068, or RLY-1971.

In some embodiments, the combination therapy comprises conjoint administration of a compound of the disclosure, such as Compound 1 (e.g., Form 2 of Compound 1), with a RAS inhibitor, such as adagrasib and sotorasib.

In some embodiments, the combination therapy comprises conjoint administration of a compound of the disclosure, such as Compound 1 (e.g., Form 2 of Compound 1), with a Trophoblast cell-surface antigen 2 (TROP2) antibody-drug conjugate (ADC). TROP2 is a cell surface transmembrane protein that is highly expressed in >50% non-small cell lung cancer, including lung adenocarcinoma with ALK rearrangement (Inamura, K. et al. Oncotarget. 2017; 8(17):28725-28735). Combination of compound 1 and TROP2-ADC may be able to provide additional therapeutic benefit than each monotherapy, especially to patients with ALK-independent resistance mechanisms. In some embodiments, the TROP2 ADC is DS-1062.

In some embodiments, the combination therapy comprises conjoint administration of a compound of the disclosure, such as Compound 1 (e.g., Form 2 of Compound 1), with an AXL ADC (e.g. enapotamab vedotin). AXL is a receptor tyrosine kinase, with increased expression associated with acquired ALK TKI resistance and epithelial-to-mesenchymal transition (Ref: Debruyne et al, Oncogene 2016). Combination of compound 1 and AXL-ADC may provide additional therapeutic benefit than each monotherapy, especially in ALK TKI resistant patients with increased AXL expression.

In some embodiments, the combination therapy comprises administration of a compound provided herein, e.g., Compound 1 (e.g., Form 2 of Compound 1), in combination with a TKI. In one embodiment, the TKI is an ALK inhibitor. In one embodiment, the ALK TKI is crizotinib, ceritinib, alectinib, brigatinib, lorlatinib, entrectinib, repotrectinib, cabozantinib, foretinib, merestinib, taletrectinib, masitinib, or ensartinib. In one embodiment, the ALK TKI is crizotinib, ceritinib, alectinib, brigatinib, or lorlatinib. In one embodiment, the ALK TKI is crizotinib. In one embodiment, the ALK TKI is ceritinib. In one embodiment, the ALK TKI is entrectinib. In one embodiment, the ALK TKI is alectinib. In one embodiment, the ALK TKI is brigatinib. In one embodiment, the ALK TKI is lorlatinib.

In some embodiments, the combination therapy comprises conjoint administration of a compound of the disclosure, such as Compound 1 (e.g., Form 2 of Compound 1), with anti-PD-1 therapy. In certain embodiments, the combination therapy comprises conjoint administration of a compound of the disclosure, such as Compound 1, with oxaliplatin. In other embodiments, the combination therapy comprises conjoint administration of a compound of the disclosure, such as Compound 1, with doxorubicin.

In certain embodiments, concomitant use (within 12 days of enrollment) of drugs that are known strong CYP3A4 inducers (such as phenobarbital, rifampin, phenytoin, carbamazepine, rifabutin, rifapentine, clevidipine, St. John's Wort) are prohibited. In certain embodiments, concomitant use (within 12 days of enrollment) of drugs that are known strong CYP3A4 inducers (such as phenobarbital, rifampin, phenytoin, carbamazepine, rifabutin, rifapentine, clevidipine, St. John's Wort) are permitted. In certain embodiments, concomitant use (within 12 days of enrollment) of drugs that are known strong CYP3A4 inducers (such as phenobarbital, rifampin, phenytoin, carbamazepine, rifabutin, rifapentine, clevidipine, St. John's Wort) are used with caution. In certain embodiments, the subject is not taking any one of strong inducers of CYP3A4, strong inhibitors of CYP3A4, sensitive substrates of CYP3A4 and/or CYP2C8, substrates of P-gp/multidrug resistance protein (MDR1), substrates of BCRP/breast cancer resistance protein (ABCG2), substrates of OATP1B1, substrates of OATP1B3, substrates of MATE1, or gastric acid reducing agents.

In certain embodiments, concomitant use (within 12 days of enrollment) of drugs that are strong inhibitors of CYP3A4, sensitive substrates of CYP3A4 and/or CYP2C8, substrates of P-glycoprotein (P-gp)/multidrug resistance protein 1 (MDR1), substrates of BCRP/breast cancer resistance protein (ABCG2), substrates of OATP1B1, substrates of OATP1B3, or substrates of MATE1 are used with caution. In certain embodiments, concomitant use (within 12 days of enrollment) or co-administration of Compound 1 and any one or more drugs that are strong inhibitors of CYP3A4, sensitive substrates of CYP3A4 and/or CYP2C8, substrates of P-glycoprotein (P-gp)/multidrug resistance protein 1 (MDR1), substrates of BCRP/breast cancer resistance protein (ABCG2), substrates of OATP1B1, substrates of OATP1B3, or substrates of MATE1 are permitted. In certain embodiments, concomitant use (within 12 days of enrollment) or co-administration of drugs that are strong inhibitors of CYP3A4, sensitive substrates of CYP3A4 and/or CYP2C8, substrates of P-glycoprotein (P-gp)/multidrug resistance protein 1 (MDR1), substrates of BCRP/breast cancer resistance protein (ABCG2), substrates of OATP1B1, substrates of OATP1B3, or substrates of MATE1 are prohibited. In certain embodiments, the subject is taking any one of strong inducers of CYP3A4, strong inhibitors of CYP3A4, sensitive substrates of CYP3A4 and/or CYP2C8, substrates of P-gp/multidrug resistance protein (MDR1), substrates of BCRP/breast cancer resistance protein (ABCG2), substrates of OATP1B1, substrates of OATP1B3, substrates of MATE1, or gastric acid reducing agents. In certain embodiments, the subject is taking any one of strong inducers of CYP3A4 or strong inhibitors of CYP3A4.

In certain embodiments, concomitant use (within 12 days of enrollment) or co-administration of gastric acid reducing agents with Compound 1 should be avoided. In certain embodiments, concomitant use (within 12 days of enrollment) or co-administration of gastric acid reducing agents with Compound 1 should be used with caution. In certain embodiments, concomitant use (within 12 days of enrollment) or co-administration of gastric acid reducing agents with Compound 1 is permitted.

In certain embodiments, a compound of the disclosure may be conjointly administered with non-chemical methods of cancer treatment. In certain embodiments, a compound of the disclosure may be conjointly administered with radiation therapy. In certain embodiments, a compound of the disclosure may be conjointly administered with surgery, with thermoablation, with focused ultrasound therapy, with cryotherapy, or with any combination of these.

In certain embodiments, compounds of the disclosure may be conjointly administered with one or more other compounds of the disclosure. Moreover, such combinations may be conjointly administered with other therapeutic agents, such as other agents suitable for the treatment of cancer, immunological or neurological diseases, such as the agents identified above. In certain embodiments, conjointly administering one or more additional chemotherapeutic agents with a compound of the disclosure provides a synergistic effect. In certain embodiments, conjointly administering one or more additional chemotherapeutic agents provides an additive effect. In certain embodiments, the chemotherapeutic agents are alkylating agents, antimetabolites, anti-tumor antibiotics, topoisomerase inhibitors, platinum drugs, mitotic inhibitors, hormone therapies, or corticosteroids. In some embodiments, Compound 1 (e.g. Form 2) may be conjointly administered with a pain therapy. In some embodiments, the pain therapy is an anti-inflammatory such as a Non-Steroidal Anti-Inflammatory Drug (NSAID). In some embodiments, the NSAID is ibuprofen, naproxen, diclofenac, celecoxib, mefenamic acid, etoricoxib, indomethacin, aspirin.

### Pharmaceutical Compositions

In certain embodiments, provided herein is a pharmaceutical preparation suitable for use in a human subject, comprising a compound provided herein (e.g., Compound 1, and one or more pharmaceutically acceptable excipients. In certain embodiments, the pharmaceutical preparations may be for use in treating or preventing a condition or disease as described herein. A compound provided herein may be used in the manufacture of medicaments for the treatment of any diseases or conditions disclosed herein.

The compositions and methods provided herein may be utilized to treat a subject in need thereof. In certain embodiments, the subject is a mammal such as a human, or a non-human mammal. When administered to subject, such as a human, the composition or the compound is preferably administered as a pharmaceutical composition comprising, for example, a compound provided herein and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound provided herein. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound provided herein. Liposomes, for example, which comprise phospholipids or other lipids, are nontoxic, physiologically acceptable and metabolizable carriers that are relatively simple to make and administer.

In one embodiment, provided herein is a pharmaceutical composition comprising

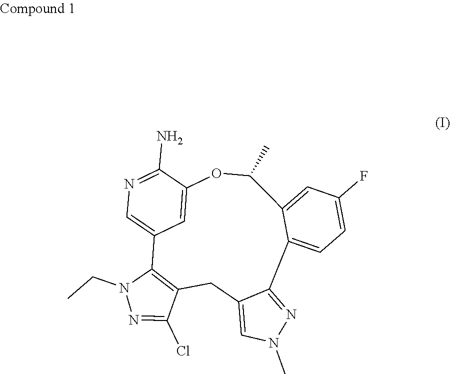 [SMILES: CCn1nc(Cl)c2c1-c1cnc(N)c(c1)O[C@H](C)c1cc(F)ccc1-c1nn(C)cc1C2]

or a stereoisomer, or a mixture of stereoisomers thereof, or a pharmaceutically acceptable salt thereof, a diluent, a disintegrant, a glidant, a binder, and a lubricant.

In one embodiment, Compound 1, or a stereoisomer, or a mixture of stereoisomers thereof, or a pharmaceutically acceptable salt thereof, is free base of Compound 1. In one embodiment, the free base of Compound 1 is amorphous. In one embodiment, the free base of Compound 1 is a crystalline free base of Compound 1. In one embodiment, the free base of Compound 1 is one of the solid forms of free base of Compound 1 provided herein. In one embodiment, the free base of Compound 1 is Form 2 of the free base of Compound 1. In one embodiment, the free base of Compound 1 is characterized by an XRPD pattern comprising peaks at approximately 12.4, 18.9, and 21.1° 2θ (±0.2°).

In one embodiment, Compound 1, or a stereoisomer, or a mixture of stereoisomers thereof, or a pharmaceutically acceptable salt thereof, in the pharmaceutical composition is a pharmaceutically acceptable salt of Compound 1. In one embodiment, the salt is amorphous.

In one embodiment, the diluent is microcrystalline cellulose. In one embodiment, the disintegrant is croscarmellose sodium. In one embodiment, the glidant is colloidal silica dioxide. In one embodiment, the binder is hydroxypropyl cellulose (HPC). In one embodiment, the lubricant is magnesium stearate.

The pharmaceutical compositions may conveniently be presented in unit dosage form. In one embodiment, the pharmaceutical composition is an oral dosage form. In one embodiment, the oral dosage form is a tablet. In certain embodiments, the unit dosage form is a tablet of 5 mg (by weight of free base Compound 1) dose strength. In certain embodiments, the unit dosage form is a tablet of 25 mg (by weight of free base Compound 1) dose strength. In certain embodiments, the unit dosage form is a tablet of 50 mg (by weight of free base Compound 1) dose strength. In certain embodiments, the unit dosage form is a tablet of 75 mg (by weight of free base Compound 1) dose strength. In certain embodiments, the unit dosage form is a tablet of 100 mg (by weight of free base Compound 1) dose strength. In certain embodiments, the unit dosage form is a tablet of 125 mg (by weight of free base Compound 1) dose strength. In certain embodiments, the unit dosage form is a tablet of 150 mg (by weight of free base Compound 1) dose strength. The amount of active ingredient which can be combined with a carrier material to produce a single dosage form will vary depending upon the subject being treated, the particular mode of administration. In one embodiment, the oral dosage form is an immediate release tablet. In one embodiment, the pharmaceutical composition is film-coated.

In certain embodiments, the present disclosure provides a pharmaceutical preparation suitable for use in a human patient, comprising any of the compounds shown above (e.g., a compound of the disclosure, such as Compound 1, and one or more pharmaceutically acceptable excipients. In certain embodiments, the pharmaceutical preparations may be for use in treating or preventing a condition or disease as described herein. Any of the disclosed compounds may be used in the manufacture of medicaments for the treatment of any diseases or conditions disclosed herein.

In certain embodiments, provided is a pharmaceutical composition comprising Form 2 and a pharmaceutically acceptable carrier. In certain embodiments, provided is a pharmaceutical composition comprising Form 2 substantially free (e.g., less than about 0.5 wt %, about 0.4 wt %, about 0.3 wt %, about 0.2 wt %, about 0.1 wt %, about 0.05 wt %, or about 0.01 wt %) of impurities. In certain embodiments, the pharmaceutical composition comprising Form 2 is substantially free of other crystal forms of Compound 1.

The phrase “pharmaceutically acceptable” is employed herein to refer to those compounds, materials, compositions, and/or dosage forms which are, within the scope of sound medical judgment, suitable for use in contact with the tissues of a subject without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4, 172,896, as well as in patents cited therein.

The formulations may conveniently be presented in unit dosage form and may be prepared by any methods well known in the art of pharmacy. In certain embodiments, the unit dosage form is a tablet. In certain embodiments, the unit dosage form is a tablet of 5 mg (by weight of free base Compound 1) dose strength. In certain embodiments, the unit dosage form is a tablet of 50 mg (by weight of free base Compound 1) dose strength. The amount of active ingredient which can be combined with a carrier material to produce a single dosage form will vary depending upon the subject being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound provided herein, with the carrier and, optionally, one or more accessory ingredients. In general, the formulations are prepared by uniformly and intimately bringing into association a compound provided herein with liquid carriers, or finely divided solid carriers, or both, and then, if necessary, shaping the product.

Formulations provided herein suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound provided herein as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound provided herein to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this disclosure. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatible with such fluids. A preferred route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions provided herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods provided herein, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular subject, composition, and mode of administration, without being toxic to the subject.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the subject being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the subject's condition, the disorder being treated, the stability of the compound, and, if desired, another type of therapeutic agent being administered with the compound provided herein. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

If desired, the effective daily dose of the active compound may be administered as one, two, three, four, five, six or more sub-doses administered separately at appropriate intervals throughout the day, optionally, in unit dosage forms. In certain embodiments provided herein, the active compound may be administered two or three times daily. In preferred embodiments, the active compound will be administered once daily.

In certain embodiments, compounds provided herein may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the subject, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, a subject who receives such treatment can benefit from a combined effect of different therapeutic compounds.

In certain embodiments, conjoint administration of compounds provided herein with one or more additional therapeutic agent(s) (e.g., one or more additional chemotherapeutic agent(s)) provides improved efficacy relative to each individual administration of the compound provided herein or the one or more additional therapeutic agent(s). In certain such embodiments, the conjoint administration provides an additive effect, wherein an additive effect refers to the sum of each of the effects of individual administration of the compound provided herein and the one or more additional therapeutic agent(s).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

The disclosure now being generally described, it will be more readily understood by reference to the following examples which are included merely for purposes of illustration of certain aspects and embodiments of the present disclosure, and are not intended to limit the disclosure.

### EXAMPLES

### Example 1: Phase 1/2 Clinical Study

A FIH, Phase 1/2, multicenter, open-label, dose escalation and expansion study is conducted to evaluate the safety and tolerability of Compound 1, to determine the RP2D, and, if applicable, the MTD, and to evaluate the antitumor activity in patients with advanced ALK-positive NSCLC and other advanced ALK-positive solid tumors.

### Phase 1 Objectives:

Primary Objective: to determine the RP2D and/or maximum tolerated dose (MTD) of Compound 1 in patients with advanced ALK-positive solid tumors.
Secondary Objectives: to evaluate the overall safety and tolerability of Compound 1; to characterize the PK profile of Compound 1; to evaluate preliminary antitumor activity of Compound 1 in patients with advanced ALK positive solid tumors.
Exploratory Objectives: to evaluate additional tumor and blood-based molecular markers of response and resistance to Compound 1; to evaluate the metabolite profile of Compound 1; to evaluate extracranial activity.

### Phase 2 Objectives:

Primary Objective: to evaluate the efficacy of Compound 1 at the RP2D in patients with advanced ALK-positive NSCLC, including those with ALK resistance mutations, and other solid tumors.
Secondary Objectives: to assess additional measures of clinical efficacy in patients with ALK-positive NSCLC, including those with ALK resistance mutations, and other solid tumors; to evaluate the intracranial antitumor activity of Compound 1 at the RP2D in patients with advanced ALK-positive NSCLC and other solid tumors; to characterize the safety and tolerability of Compound 1 at the RP2D; to confirm the PK profile of Compound 1 at the RP2D.
Exploratory Objectives: to evaluate additional tumor and blood-based molecular markers of response and resistance to Compound 1; to assess progression-free survival on subsequent therapy; to evaluate extracranial activity.

### Phase 1 Endpoints:

Primary Endpoint: RP2D and, if applicable, the MTD as determined by incidence of dose-limiting toxicities (DLTs) during Cycle 1, overall safety profile, PK, PD, and preliminary efficacy
Secondary Endpoints:
   Incidence and severity of treatment-emergent adverse events (TEAEs) and changes in clinically relevant laboratory parameters
   Pharmacokinetic parameters of Compound 1: Maximum plasma concentration (Cₘₐₓ); Cₘₐₓ—dose normalized, plasma concentration at the end of the dosing interval (Cₜₐᵤ); average plasma concentration (C_{avg}); time of maximum concentration (Tₘₐₓ); area under the curve at the end of the dosing interval (AUCₜₐᵤ); AUCₜₐᵤ—dose normalized, area under the curve from time 0 to 24 (AUC₀₋₂₄); AUC₀₋₂₄—dose normalized, area under the curve from time 0 to infinity (AUC_{inf}); AUC_{inf}—dose normalized; oral clearance (CL/F); volume of distribution (V_{z}/F); and half-life (t_{1/2})
   Objective response rate (ORR) per Response Evaluation Criteria in Solid Tumors Version 1.1 (RECIST 1.1)—Defined as the percent of patients with a complete response (CR) or partial response (PR) according to RECIST 1.1
   Duration of response (DOR) per RECIST 1.1—In responders, defined as the time from first response per RECIST 1.1 to radiographic disease progression or death
   Intracranial ORR (IC-ORR) per RECIST 1.1—In patients with measurable metastatic CNS disease up to 5 intracranial target lesions are accessed for response, according to RECIST 1.1
   Intracranial DOR (IC-DOR) per RECIST 1.1—In responders with metastatic CNS disease, defined as the time from first IC-response per RECIST 1.1 to radiographic IC-disease progression or death
   Clinical benefit rate (CBR) per RECIST 1.1—Defined as the percent of patients with a confirmed CR or PR, or stable disease (SD) of at least 24 weeks duration according to RECIST 1.1
   Time to response per RECIST 1.1—Defined as the time from first dose to first confirmed radiographic response according to RECIST 1.1
   Progression-free survival (PFS) per RECIST 1.1—Defined as the time from first dose to radiographic disease progression per RECIST 1.1 or death
Exploratory Endpoints:
   Baseline levels and/or post-treatment changes in tumor and blood biomarker levels including but not limited to expression of proteins, phospho-proteins, genes, variant allele frequency, and genetic variations
   Compound 1 metabolite identification and quantification in post-treatment blood samples
   Extracranial-ORR: In patients with extracranial metastatic disease, defined as the percent of patients with an extracranial response according to RECIST 1.1 per Investigator assessment
Phase 2 Endpoints:
   Primary Endpoint: ORR per RECIST 1.1—Defined as the proportion of patients with a confirmed CR or PR according to RECIST 1.1 per Blinded Independent Central Review (BICR) of the patients confirmed to have measurable disease at baseline per BICR.
Secondary Endpoints
   DOR per RECIST 1.1—In responders, defined as the time from first BICR-assessed confirmed response per RECIST 1.1 to radiographic disease progression or death
   CBR per RECIST 1.1—Defined as the percent of patients with a confirmed CR or PR, or SD of at least 24 weeks duration according to RECIST 1.1 per BICR
   Time to response per RECIST 1.1—Defined as the time from first dose to first confirmed radiographic response according to RECIST 1.1 per BICR
   PFS per RECIST 1.1—Defined as the time from first dose to radiographic disease progression per RECIST 1.1 based on BICR assessment or death
   Overall survival (OS)—Defined as the time from first dose to death due to any cause
   IC-ORR per RECIST 1.1—In patients with measurable metastatic CNS disease up to 5 intracranial target lesions are assessed for response according to RECIST 1.1 per BICR of the patients confirmed to have measurable intracranial disease at baseline per BICR
   IC-DOR per RECIST 1.1—In responders with metastatic CNS disease, defined as the time from first BICR-assessed confirmed response per RECIST 1.1 to radiographic disease progression or death.
   Time to IC-response per RECIST 1.1—In patients with metastatic CNS disease, defined as the time from first dose to first confirmed radiographic IC-response according to RECIST 1.1 per BICR
   Intracranial PFS (IC-PFS) per RECIST 1.1—In patients with metastatic CNS disease, defined as the time from first dose to radiographic disease progression per RECIST 1.1 based on BICR assessment or death
   Incidence and severity of TEAEs and changes in clinically relevant laboratory parameters
   Pharmacokinetic parameters of Compound 1—Cₘₐₓ, Cₘₐₓ—dose normalized, Cₜₐᵤ, C_{avg}, Tₘₐₓ, AUCₜₐᵤ, AUCₜₐᵤ—dose normalized, AUC₀₋₂₄, AUC₀₋₂₄—dose normalized, AUC_{inf}, AUC_{inf}—dose normalized, CL/F, V_{z}/F, t_{1/2}
Exploratory Endpoints:
   Baseline levels and/or post-treatment changes in tumor and blood biomarker levels including but not limited to expression of proteins, phospho-proteins, genes, variant allele frequency, and genetic variations
   PFS2: Defined as the time from beginning of treatment with Compound 1 until disease progression on subsequent therapy (based on local assessment or medical record report of progression on subsequent therapy) or death due to any cause
   EC-ORR: In patients with extracranial metastatic disease, defined as the percent of patients with an extracranial response according to RECIST 1.1 per Investigator assessment

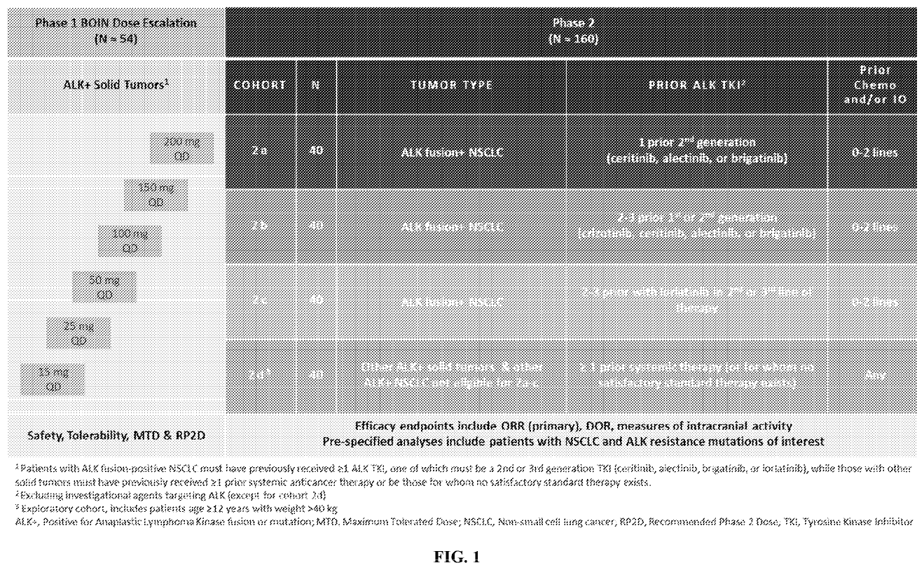

Study Design: The study is conducted in 2 phases (FIG. 1).

The Phase 1 dose-escalation includes patients with locally advanced or metastatic solid tumors harboring an ALK rearrangement or activating ALK mutation. Patients with ALK fusion-positive NSCLC must have previously received ≥1 ALK TKI, one of which must be a 2nd or 3^{rd} generation TKI (ceritinib, alectinib, brigatinib, or lorlatinib), while those with other solid tumors must have previously received ≥1 prior systemic anticancer therapy or be those for whom no satisfactory standard therapy exists. The Phase 1 portion of the study is designed to determine the RP2D and, if applicable, the MTD and to assess the safety of Compound 1.

The Phase 1 portion of the study employs a BOIN design with a 3+3 run-in to evaluate dose escalation. The DLT observation period is defined from first dose on C1D1 through to the end of the first cycle (21-day cycle period). After each group of patients are treated at a given dose level and monitored for 21 days, available data (to include, but not limited to, the incidence of DLT/other AEs and preliminary PK) are reviewed. Initiation of the next dosing group depends on the occurrence of DLT, outcome of the BOIN analysis, and the recommendation with consideration to the overall safety profile. The BOIN dose-escalation is declared complete when the number of evaluable patients treated at the current dose reaches 12 and the decision is to stay at the current dose, or when the maximum sample size is reached. However, if an optimal biologic dose or maximal feasible dose is reached prior to the completion of the BOIN dose-escalation, the RP2D may be declared. Patients with a DLT are not replaced. Patients who are unevaluable for DLT (do not receive ≥16 days of intended dosing and/or do not complete the 21-day observation period for reasons other than DLT during the first cycle) may be replaced.

After a DLT has occurred, if continued treatment with Compound 1 is determined to be in the patient's interest, treatment with Compound 1 may resume at a reduced dose. If the AE resulting in dose interruption meets the definition of a DLT, then study drug is resumed at the previous safe dose level or reduced dose level (see Table A for recommended reduced dose levels). Upon resolution of the AE and dose interruption, the patient may resume therapy with a reduction of 1 dose level. If it is determined to be in the interest of the patient, study treatment my resume without dose reduction. If the AE recurs with same or worsening intensity, treatment is interrupted, and the dose is reduced should study treatment continue. A maximum of 3 dose reductions are permitted for any patient.

TABLE A
Recommended Dose Reduction Levels
(After DLT Observation Period)

| | | |
|---|---|---|
| Initial dose level | Dose level -1 | Dose level -2** |

| 15 | mg QD | 10 | mg QD | NA | |
|---|---|---|---|---|---|
| 25 | mg QD | 15 | mg QD | 10 | mg QD |
| 50 | mg QD | 25 | mg QD | 15 | mg QD |
| 100 | mg QD | 50 | mg QD | 25 | mg QD |
| 150 | mg QD | 100 | mg QD | 50 | mg QD |
| 200 | mg QD* | 150 | mg QD | 100 | mg QD |

Abbreviations: NA, not applicable; QD: once daily.
*If dose reductions are needed, the dose should be reduced to the lower dose level.
**If treatment is not tolerated after 2 dose reductions, a third dose reduction may be implemented if it is in the best interest of the patient and upon mutual agreement between the Investigator and Sponsor, or treatment may be discontinued.

During dose escalation, when available data support the safety, PK, and clinical activity of a given dose level, additional patients (up to a total of 12 patients, including patients already included in BOIN dose escalation) may be enrolled at that dose level. These patients are monitored for DLT and included in the evaluation for the conduct of the BOIN dose-escalation. A total of up to approximately 54 patients may be treated in the Phase 1 dose-escalation.

The selection of the RP2D occurs after a sufficient number of patients have enrolled and completed the first imaging/efficacy assessment and the data have been reviewed. The RP2D is expected to be an MTD or a lower optimal biological dose, or a maximum feasible dose, and is the dose level deemed as suitable for testing in the Phase 2 portion of the study. The selection of the RP2D is made using the totality of the data obtained from Phase 1, and includes the evaluation of clinical PK, pharmacodynamics, safety, and antitumor activity.

### Phase 2 Includes 4 Cohorts:

Cohort 2a: Patients with locally advanced or metastatic NSCLC harboring an ALK rearrangement who have received 1 prior 2^{nd} generation ALK TKI (ceritinib, alectinib, or brigatinib).
Cohort 2b: Patients with locally advanced or metastatic NSCLC harboring an ALK rearrangement, who have received 2-3 prior 1^{st} or 2^{nd} generation ALK TKIs (crizotinib, ceritinib, alectinib, or brigatinib).
Cohort 2c: Patients with locally advanced or metastatic NSCLC harboring an ALK rearrangement, who have received 2-3 prior 1^{st} or 2^{nd} generation ALK TKIs (crizotinib, ceritinib, alectinib, or brigatinib).
Cohort 2d: Patients with other solid tumors harboring an ALK rearrangement or activating ALK mutation, including patients with NSCLC not eligible for cohorts 2a-c, who have received ≥1 prior systemic anticancer therapy, or for whom no satisfactory standard therapy exists.

The Phase 2 portion of the study is designed to estimate the ORR and the corresponding 95% CI for the following populations of patients: all treated patients with ALK-positive NSCLC pooled across cohorts 2a, 2b and 2c; each individual cohort 2a, 2b and 2c; and patients with ALK resistance mutations of interest (i.e., any ALK resistance mutation; the ALK G1202R mutation, whether occurring with or without other ALK mutations) pooled across cohorts 2a, 2b and 2c.

Phase 1 Dose Levels: Phase 1 evaluates ascending doses of Compound 1 monotherapy administered orally once daily (QD) continuously until disease progression, unacceptable toxicity, or voluntary withdrawal of consent. Under the BOIN design, up to approximately 54 patients are enrolled and treated at the dose levels presented in the following table.

| Dose level | Compound 1 Dose | | Frequency* |
|---|---|---|---|
| DL1 | 15 | mg | QD |
| DL2 | 25 | mg | QD |
| DL3 | 50 | mg | QD |
| DL4 | 100 | mg | QD |
| DL5 | 150 | mg | QD |
| DL6** | 200 | mg | QD |

*BID dosing may be explored at any dose level (“DL”).
**Higher DLs may be explored.

Intermediate dose levels may be explored during dose escalation; a lower dose level (e.g., 10 mg QD) may be explored based on results of the BOIN.

Recommended Phase 2 Dose: The recommended Phase 2 dose (RP2D) is expected to be an MTD or a lower optimal biological dose, or a maximum feasible dose, and is the dose level deemed as suitable for testing in the Phase 2 portion of the study. The selection of the RP2D is made using the totality of the data obtained from Phase 1, and includes the evaluation of clinical PK, pharmacodynamics, safety, and antitumor activity.

Duration of Treatment: Across both phases, patients receive study drug continuously from first dose until independent radiological confirmation of disease progression, unacceptable toxicity, withdrawal by patient, termination the study, or commercial availability. Patients may continue to receive Compound 1 following progression in the body or brain suitable for local ablation.

End of Study Definition: The end of the study is defined as the date of the last visit of the last patient in the study or termination of the study.

Inclusion Criteria: Patients must meet all of the following criteria to be eligible to enroll in the study:
1. Age ≥18 years. (Phase 2 Cohort 2d only: Age ≥12 years and weighing >40 kg.)
2. Disease criteria
   a. Phase 1: Histologically or cytologically confirmed locally advanced or metastatic solid tumor with a documented ALK rearrangement or activating ALK mutation detected by certified assay (i.e. CLIA in the US).
   b. Cohorts 2a, 2b, and 2c: Histologically or cytologically confirmed locally advanced or metastatic NSCLC with a documented ALK rearrangement detected by certified assay (i.e. CLIA in the US).
   c. Cohort 2d: Any other histologically or cytologically confirmed locally advanced or metastatic solid tumor with a documented ALK rearrangement or activating ALK mutation detected by certified assay (i.e. CLIA in the US), including but not limited to anaplastic large cell lymphoma, inflammatory myofibroblastic tumors, diffuse large B-cell lymphoma, esophageal squamous cell carcinoma, renal medullary carcinoma, renal cell carcinoma, breast cancer, colorectal cancer, ovarian cancer, papillary thyroid carcinoma, cholangiocarcinoma, spitzoid tumors, neuroblastoma, anaplastic thyroid cancer, and patients with NSCLC not eligible for Cohorts 2a-c.
3. Prior anticancer treatment
   a. Phase 1: Patients with ALK fusion-positive NSCLC must have previously received ≥1 ALK TKI, one of which must be a 2^{nd} or 3^{rd} generation TKI (ceritinib, alectinib, brigatinib, or lorlatinib). Patients with other solid tumors must have previously received ≥1 prior systemic anticancer therapy or be those for whom no satisfactory standard therapy exists.
   b. Cohort 2a: 1 prior 2^{nd} generation ALK TKI (ceritinib, alectinib, or brigatinib).
   c. Cohort 2b: 2-3 prior 1^{st} or 2^{nd} generation ALK TKIs (crizotinib, ceritinib, alectinib, or brigatinib).
   d. Cohort 2c: 2-3 prior ALK TKIs, with lorlatinib received in the 2^{nd} or 3^{rd} line of therapy.
   e. All patients except Phase 2 Cohort 2d: ≤2 prior lines of chemotherapy and/or immunotherapy in the locally advanced or metastatic setting. Patients who have received >2 prior lines of chemotherapy and/or immunotherapy in the locally advanced or metastatic setting may be enrolled in Phase 2 Cohort 2d.
   f. Phase 2 Cohorts 2a, 2b, and 2c: No prior investigational agents targeting ALK. Patients who previously received investigational agents targeting ALK may be enrolled in Phase 1 and Phase 2 Cohort 2d.
4. Phase 1: Must have evaluable disease (target or nontarget) according to RECIST 1.1. Phase 2: Must have measurable disease, defined as ≥1 radiologically measurable target lesion according to RECIST 1.1 (Oken et al., Am. J. Clin. Oncol. 1982, 5(6):649-655).
5. Pre-treatment tumor tissue (archived, if available, or a fresh biopsy) submitted for central analysis. It is preferable that submitted tumor tissue be obtained during or after the most recent disease progression. If appropriate tissue is not available, and if biopsy is not considered safe and medically feasible by the Investigator, the patient may be approved for enrollment after consultation with the Sponsor's Medical Monitor.
6. Eastern Cooperative Oncology Group (ECOG) performance status (PS) of 0, 1 or 2 (Eisenhauer et al., Eur. J. Cancer 2009, 45(2):228-247).
7. Adequate organ function and bone marrow reserve as indicated by the following laboratory assessments performed prior to the first dose of study drug:
   a. Bone marrow function: absolute neutrophil count (ANC)≥1500/μL; platelet count >75,000/μL; hemoglobin ≥8 g/dL.
   b. Renal function: estimated creatinine clearance ≥60 mL/min Hepatic function: bilirubin <1.5×ULN, unless evidence of Gilbert Syndrome, in which the patient must have total bilirubin ≤3.0 mg/dL; aspartate aminotransferase and alanine aminotransferase≤3.0×ULN (≤5.0×ULN if liver metastases involvement).
8. All clinically relevant toxicities related to prior anticancer therapy must have recovered to Grade ≤1 or baseline (except alopecia or ototoxicity).
9. Women of childbearing potential (WOCBP) and male patients must be willing to abstain from sexual activity or use an effective contraceptive method or be surgically sterile from the time of signing the informed consent form (ICF) through the duration of the study and for 30 days for women and 90 days for men after the last administration of study drug. Effective contraception for WOCBP includes 1 “highly effective method” or 2 “effective” methods based on World Health Organization (WHO) criteria.
10. Provide written informed consent and willing and able to comply with requirements of the study protocol. Assent must be obtained for patients <18 years old (Phase 2 Cohort 2d only) and a parent/guardian must provide written consent.

Exclusion Criteria: Patients meeting any of the following criteria are excluded from the study:
1. Patient's cancer has a known primary driver alteration other than ALK.
2. Known allergy/hypersensitivity to excipients of Compound 1.
3. Major surgery within 4 weeks of study entry. Minor surgical procedures (e.g., port insertion) are permitted, but with sufficient time for wound healing as deemed clinically appropriate.
4. Ongoing or recent anticancer therapy within the following timeframe prior to first dose of study drug (Compound 1 may be started within limits for prior TKI or chemotherapy if considered to be safe and within the best interest of the patient, with prior approval):
   a. TKI or other non-chemotherapy/non-immunotherapy anticancer agents <5 half-lives or <7 days, whichever is longer
   b. Chemotherapy <21 days
   c. Immunotherapy or cellular therapy <28 days
5. Ongoing or recent radiation therapy within the following timeframe prior to first dose of study drug:
   a. Radiation therapy (except palliative radiation to relieve bone pain)<14 days
   b. Palliative radiation to relieve bone pain <48 hours
   c. Stereotactic or small field brain irradiation <7 days
   d. Whole brain radiation <14 days
6. Prior high-dose chemotherapy requiring stem cell rescue.
7. Uncontrolled clinically relevant bacterial or fungal infection requiring systemic therapy.
8. Has known active tuberculosis or active Hepatitis B or C. Active Hepatitis B is defined as a known positive HBsAg result and known quantitative HBV DNA results greater than the lower limits of detection of the assay. Active Hepatitis C is defined by a known positive Hep C Ab result and known quantitative HCV RNA results greater than the lower limits of detection of the assay.
9. Patient has a QTcF consistently >450 msec. Patient has a history of prolonged QT syndrome or Torsades de pointes.
10. Patients with clinically significant cardiovascular disease as follows:
   a. Within 3 months of enrollment: cerebral vascular accident/stroke; myocardial infarction; unstable angina; uncontrolled atrial fibrillation of any grade
   b. History of congestive heart failure (New York Heart Association Classification Class ≥II); second-degree or third-degree atrioventricular block (unless paced) or any atrioventricular block with PR consistently >220 msec; or ongoing cardiac dysrhythmias of NCI-CTCAE Grade ≥2.
11. Patient has central nervous system (CNS) metastases or a primary CNS tumor that is associated with progressive neurological symptoms or requires increasing doses of corticosteroids to control the CNS disease. If a patient requires corticosteroids for management of CNS disease, the dose must have been stable for the 2 weeks preceding C1D1. Asymptomatic leptomeningeal carcinomatosis is allowed.
12. Symptomatic spinal cord compression.
13. Patients with moderate to severe cognitive impairment or psychiatric disturbances that would compromise the patient's ability to comply with study requirements.
14. Evidence of active malignancy (other than current ALK-positive solid malignancy) requiring systemic therapy within the prior 2 years.
   a. Exceptions: nonmelanoma skin cancer, in situ melanoma, in situ cervical cancer, papillary thyroid cancer, ductal carcinoma in situ of the breast, or localized and presumed cured prostate cancer. Patients on long-term anti-hormonal therapy for a prior malignancy are allowed as long as the malignancy has not been active within the prior 2 years.
15. Concomitant use (within 12 days of enrollment) of strong CYP3A4 inducers or strong CYP3A4 inhibitors.
16. Manifestation of malabsorption due to prior gastrointestinal surgery, disease, or other illness that could affect oral absorption, distribution, metabolism, or excretion of the study drug.
17. Patient is pregnant or breast-feeding. WOCBP must have a negative serum pregnancy test at Screening and negative serum or urine tests prior to first dose of study drug.
18. Actively receiving systemic treatment or direct medical intervention on another therapeutic clinical study.
19. Any evidence of current ILD or pneumonitis or a prior history of ILD or non-infectious pneumonitis.
20. Any medical condition or laboratory abnormality that would pose a risk to study patient or confound the ability to interpret study results.

Study Drug, Dosage, and Route of Administration: Compound 1 is supplied as tablets for oral administration in 2 strengths: 5 mg and 50 mg. Compound 1 should be taken on an empty stomach, at least 1 hour before and no sooner than 2 hours after ingestion of food and/or beverages other than water.

### Prior and Concomitant Medications and Therapies:

In certain embodiments, concomitant use (within 12 days of enrollment) of drugs that are known strong CYP3A4 inducers (such as phenobarbital, rifampin, phenytoin, carbamazepine, rifabutin, rifapentine, clevidipine, St. John's Wort) are prohibited. In certain embodiments, concomitant use (within 12 days of enrollment) of drugs that are known strong CYP3A4 inducers (such as phenobarbital, rifampin, phenytoin, carbamazepine, rifabutin, rifapentine, clevidipine, St. John's Wort) are permitted. In certain embodiments, concomitant use (within 12 days of enrollment) of drugs that are known strong CYP3A4 inducers (such as phenobarbital, rifampin, phenytoin, carbamazepine, rifabutin, rifapentine, clevidipine, St. John's Wort) are used with caution.

In certain embodiments, concomitant use (within 12 days of enrollment) of drugs that are known strong CYP3A4 inhibitors are prohibited. In certain embodiments, concomitant use (within 12 days of enrollment) of drugs that are known strong CYP3A4 inhibitors are permitted. In certain embodiments, concomitant use (within 12 days of enrollment) of drugs that are known strong CYP3A4 inhibitors are used with caution. In certain embodiments, the strong CYP3A4 inhibitors are clarithromycin, erythromycin, diltiazem, itraconazole, ketoconazole, ritonavir, verapamil, Goldenseala, and Grapefruitb (including juices).

In certain embodiments, concomitant use (within 12 days of enrollment) of drugs that are strong inhibitors of CYP3A4, sensitive substrates of CYP3A4 and/or CYP2C8, substrates of P-glycoprotein (P-gp)/multidrug resistance protein 1 (MDR1), substrates of BCRP/breast cancer resistance protein (ABCG2), substrates of OATP1B1, substrates of OATP1B3, or substrates of MATE1 are used with caution. In certain embodiments, concomitant use (within 12 days of enrollment) or co-administration of Compound 1 and any one or more drugs that are strong inhibitors of CYP3A4, sensitive substrates of CYP3A4 and/or CYP2C8, substrates of P-glycoprotein (P-gp)/multidrug resistance protein 1 (MDR1), substrates of BCRP/breast cancer resistance protein (ABCG2), substrates of OATP1B1, substrates of OATP1B3, or substrates of MATE1 are permitted. In certain embodiments, concomitant use (within 12 days of enrollment) or co-administration of drugs that are strong inhibitors of CYP3A4, sensitive substrates of CYP3A4 and/or CYP2C8, substrates of P-glycoprotein (P-gp)/multidrug resistance protein 1 (MDR1), substrates of BCRP/breast cancer resistance protein (ABCG2), substrates of OATP1B1, substrates of OATP1B3, or substrates of MATE1 are prohibited.

In certain embodiments, concomitant use (within 12 days of enrollment) or co-administration of gastric acid reducing agents with Compound 1 should be avoided. In certain embodiments, concomitant use (within 12 days of enrollment) or co-administration of gastric acid reducing agents with Compound 1 should be used with caution. In certain embodiments, concomitant use (within 12 days of enrollment) or co-administration of gastric acid reducing agents with Compound 1 is permitted.

### Exemplified Agents Provided Herein Include, but are not Limited to:

CYP3A4 Inducers: phenobarbital, rifampin, phenytoin, carbamazepine, rifabutin, rifapentine, clevidipine, St. John's Wort
CYP3A4 Inhibitors: clarithromycin, erythromycin, diltiazem, itraconazole, ketoconazole, ritonavir, verapamil, Goldenseal, Grapefruit (including juices)
CYP3A4 Substrates (sensitive): buspirone, everolimus, lovastatin, midazolam, simvastatin, triazolam, maraviroc, conivaptan, darifenacin
CYP2C8 Substrates (sensitive): repaglinide, montelukast, pioglitazone, rosiglitazone
P-gp Substrates: digoxin, fexofenadine, loperamide, quinidine, talinolol, vinblastine
BCRP Substrates: daidzein, dantrolene, estrone-3-sulfate, prazosin, sulfasalazine
OATP1B1/OATP1B3 Substrates: antiviral protease inhibitors (e.g. ritonavir), clarithromycin, cyclosporine, gemfibrozil, rifampin, simeprevir
MATE1 Substrates: metformin, tetra-ethylammonium (TEA), cimetidine, procainamide

### Example 2: Cholangiocarcinoma MR619 Model

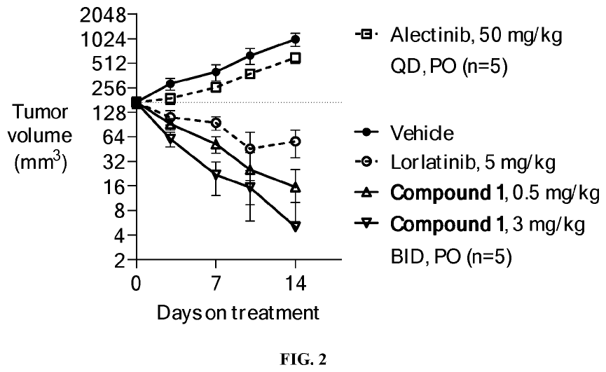

The MR619 model was derived from a cholangiocarcinoma patient with STRN-ALK fusion who progressed on alectinib treatment with acquisition of the G1202R resistance mutation. Tumors were implanted subcutaneously in 8-week-old male NOD scid gamma (NSG) mice obtained from Charles River Laboratories (France). Animals were randomized into 5 groups of 5 animals, with an average initial tumor volume of ˜170 mm³, and treated with vehicle (BID PO), alectinib (50 mg/kg QD PO), lorlatinib (5 mg/kg BID PO), Compound 1 (0.5 mg/kg BID PO), or Compound 1 (3 mg/kg BID PO) for 14 days. Vehicle is 20% HP-β-CD in water and was used to formulate Compound 1. Lorlatinib was formulated with 2 eq. HCl+20% HP-β-CD in water. Alectinib was formulated in 0.02N HCl, 10% DMSO, 10% Cremophor EL, 15% PEG-400, and 15% HP-β-CD. Tumor volume was measured twice per week, and body weight was measured about 5 times per week. Results confirmed that the model is resistant to alectinib, consistent with the clinical observation. Lorlatinib was able to induce regression, but Compound 1 at both doses induced stronger regression than lorlatinib (FIG. 2). Abbreviations: QD=once per day dosing; BID=twice per day dosing; PO=administered orally; HP-β-CD=hydroxypropyl-β-cyclodextrin; DMSO=dimethylsulfoxide; PEG=polyethyleneglycol.

### Example 3: Neuroblastoma Assays

Biochemical assay. Several kinase inhibitors including Compound 1 were evaluated for their inhibitory activity in the biochemical PhosphoSens assay (AssayQuant Technologies, MA, USA). These compounds were premixed with AT, DTT, and the fluorogenic substrate AQT0101 (AssayQuant Technologies) for 30 minutes at 30° C. Reaction was initiated by addition of one of the following enzymes: 2 nM ALK F1174L (SignalChem #A19-12EG); 0.5 nM ALK F1174S (SignalChem #A19-12FG); 1 nM ALK R1275Q (SignalChem #A19-12LG); or 0.5 nM ALK T1151M (SignalChem #A19-12BG) in reaction buffer. The final reaction conditions include 54 mM HEPES pH 7.5, 1 mM ATP, 1.2 mM DTT, 0.012% Brij-35, 0.52 mM EGTA, 1% glycerol, 0.2 mg/mL BSA, 10 mM MgCl₂, 15 μM AQT0101, and the specified concentrations of the enzymes. The plate was read by BioTek Synergy Neo2 at λ_{ex/em}=360/485 nm every 1-2 minutes for 240 minutes at 30° C. Initial rates of reaction (v) were calculated from the change in fluorescence intensity over time during the initial, linear portion of the reaction. Half-maximal inhibitory concentration (IC₅₀) was determined from percent inhibition of initial rates of reaction and inhibitor concentration using four-parameter logistic regression.

Cell viability assay. Kelly (ALK F1174L), SH-SY5Y (ALK F1174L), or NB-1 (ALK Ex2-3del) cells were plated at 1,000 cells/well (40 μL) in a 384-well plate for 1 day. Test compounds (40 nL) were then added in a 3-fold dilution series using the TECAN EVO200 liquid handler and incubated for 72 hours. Plates were equilibrated at room temperature for 15 minutes followed by addition of 40 μL CellTiter-Glo reagent (Promega). Luminescence was measured on a plate reader. Half-maximal inhibitory concentration (IC₅₀) was calculated from percent inhibition and inhibitor concentration using four-parameter logistic regression.

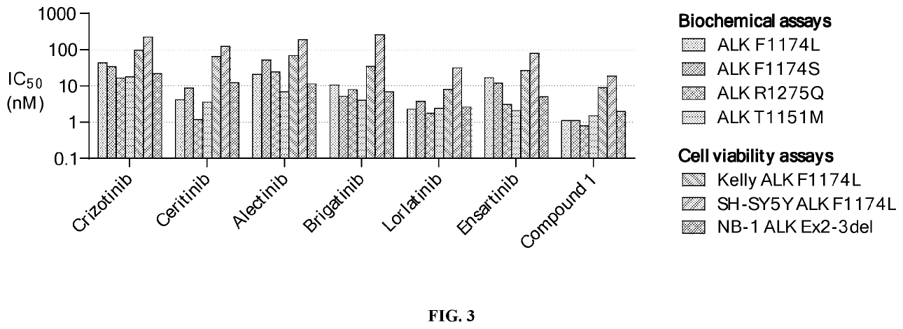

The summary of results from the biochemical assay and cell viability assay is shown in FIG. 3.

### Example 4: Anaplastic Large-Cell Lymphoma Assays

Cell viability assay. Karpas299 cells were plated at 1,000 cells/well (40 μL) in a 384-well plate for 1 day. Test compounds (each 40 nL) were then added in a 3-fold dilution series using the TECAN EVO200 liquid handler and incubated for 72 hours. Plates were equilibrated at room temperature for 15 minutes followed by addition of 40 μL CellTiter-Glo reagent (Promega). Luminescence was measured on a plate reader. Half-maximal inhibitory concentration (IC₅₀) was calculated from percent inhibition and inhibitor concentration using four-parameter logistic regression.

Cell phosphorylation assay. Test compounds (each 100 nL) were added to a 96-well plate in a 3-fold dilution series using the TECAN EVO200 liquid handler. Karpas299 cells were added at 60,000 cells/well and incubated for 6 hours. ALK phosphorylation was quantified using the PathScan Phospho-ALK (Tyr1604) Chemiluminescent Sandwich ELISA Kit (Cell Signaling Technology #7020) according to the manufacturer's instructions. Half-maximal inhibitory concentration (IC₅₀) was calculated from percent inhibition and inhibitor concentration using four-parameter logistic regression.

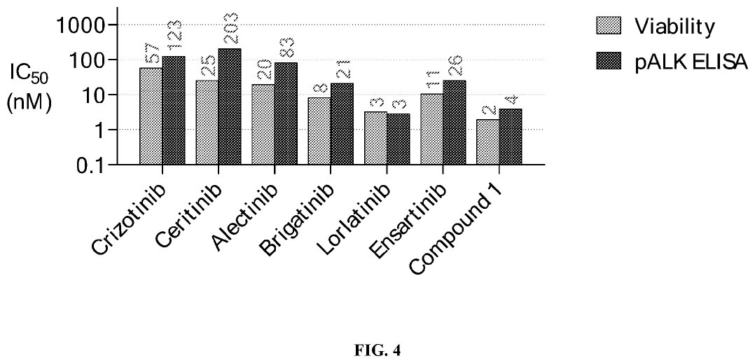

The summary of results from the cell viability assay and cell phosphorylation assay is shown in FIG. 4.

### Example 5: Soft-Tissue Sarcoma (SS) Assays

Cell viability assay. Aska-SS cells bearing ALK deletion around exons 2-17 were plated at 1,000 cells/well (40 μL) in a 384-well plate for 1 day. Test compounds (each 40 nL) were then added in a 3-fold dilution series using the TECAN EVO200 liquid handler and incubated for 72 hours. Plates were equilibrated at room temperature for 15 minutes followed by addition of 40 μL CellTiter-Glo reagent (Promega). Luminescence was measured on a plate reader. Half-maximal inhibitory concentration (IC₅₀) was calculated from percent inhibition and inhibitor concentration using four-parameter logistic regression.

Cell phosphorylation assay. Test compounds (each 100 nL) were added to a 96-well plate in a 3-fold dilution series using the TECAN EVO200 liquid handler. Aska-SS cells were added at 60,000 cells/well and incubated for 6 hours. ALK phosphorylation was quantified using the PathScan Phospho-ALK (Tyr1604) Chemiluminescent Sandwich ELISA Kit (Cell Signaling Technology #7020) according to the manufacturer's instructions. Half-maximal inhibitory concentration (IC₅₀) was calculated from percent inhibition and inhibitor concentration using four-parameter logistic regression.

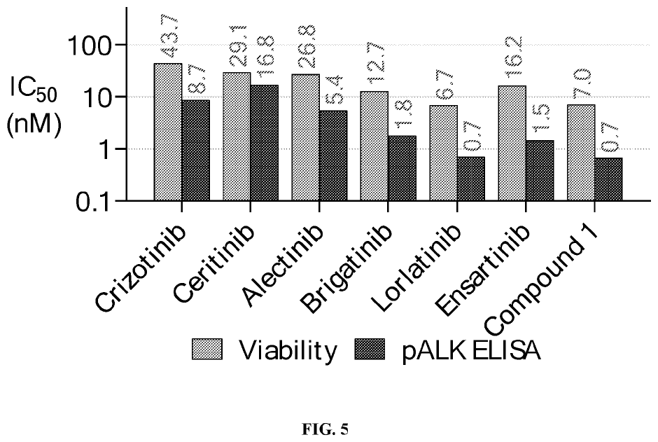

The summary of results from the cell viability assay and cell phosphorylation assay is shown in FIG. 5.

### Example 6: MR448Re Cell Line Bearing EML4-ALK v3 G1202R/T1151M Mutation

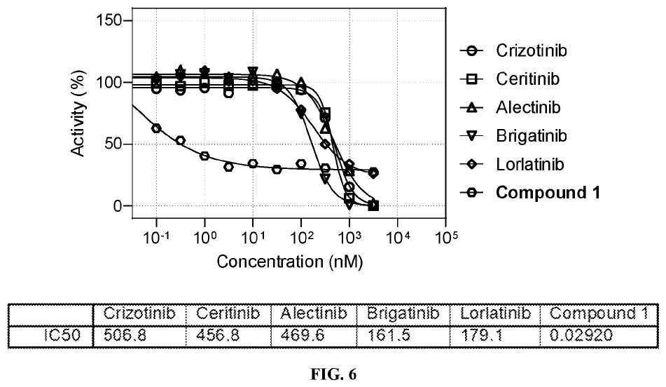

MR448re cell line was established from ascites (effusion in the peritoneal cavity) of a non-small cell lung cancer patient who harbored EML4-ALK v3 fusion and had progressed on lorlatinib treatment with acquisition of the G1202R/T1151M mutation. Ascites mononuclear cells were isolated by Ficoll centrifugation and cultured in media (DMEM F12 Glutamax, 10% antibiotics/antimycotics, 10% FBS, hydrocortisone, adenine, RockInhibitor, and 1/10 cholera toxin). After stable cancer cells were obtained, the presence of EML4-ALK fusion and G1202R/T1151M compound mutation was confirmed by PCR and sequencing. MR448re cells were treated with test compounds in a semi-log dilution series (10 μM, 3 μM, 1 μM, 300 nM, 100 nM, 30 nM, 10 nM, 3 nM, 1 nM, 0.3 nM, and 0.1 nM) for 72 hours, and viability was measured using CellTiter-Glo reagent (Promega). Half-maximal inhibitory concentration (IC₅₀) was calculated from percent inhibition and inhibitor concentration using four-parameter logistic regression. Representative data are shown in FIG. 6.

### Example 7: Mutational Evaluation

Using the following generic procedure, the inhibitory activity of ALK inhibitors was evaluated in Ba/F3 cells bearing EML4-ALK fusion with diverse resistance mutations (G1202R, G1202R/T1151M, G1202R/L1196M, G1202R/L1198F, G1202R/G1269A, I1171N, I1171S, I1171T, L1196M, or D1203N), human cell lines NCI-H2228 and NCI-H3122 which bear EML4-ALK fusion, and Ba/F3 cells bearing ETV6-TRKB or TPM3-TRKA fusion.

Cells were plated in a 384-well plate. Test compounds were added in a 3-fold dilution series using the TECAN EVO200 liquid handler and incubated for 72 hours. Plates were equilibrated at room temperature for 15 minutes followed by addition of CellTiter-Glo reagent (Promega). Luminescence was measured on a plate reader. Half-maximal inhibitory concentration (IC₅₀) was calculated from percent inhibition and inhibitor concentration using four-parameter logistic regression. Representative data are shown in FIG. 7.

### Example 8: MR448Re PDX Study

MR448re cell line was established from ascites (effusion in the peritoneal cavity) of a NSCLC patient progressing on lorlatinib. Ascites mononuclear cells were isolated by Ficoll centrifugation and cultured in media (DMEM F12 Glutamax, 10% antibiotics/antimycotics, 10% FBS, hydrocortisone, adenine, RockInhibitor, and 1/10 cholera toxin). After obtaining stable cancer cells, five million cells were injected into Swiss nude mice to derive a xenograft model. After inoculation, mice were randomized and grouped according to the tumor size. Test compounds were administered twice a day orally over a period of days.

### Example 9: Western Blot Analysis of PD Markers

Summary ALK gene rearrangements can induce constitutive activation of ALK kinase activity, leading to ALK autophosphorylation (pALK) and subsequent activation of the MAP kinase (MAPK), PI3K/AKT, and JAK/STAT pathways to drive tumor cell proliferation and survival (Shaw A T and Engelman J A. ALK in lung cancer: past, present, and future. J Clin Oncol 2013; 31 (8): 1105-1111). pALK was evaluated as a proximal PD marker, along with phospho-p44/42 MAPK ERK1/2 (PERK) as a downstream MAPK pathway marker, phospho-AKT (pAKT) and phospho-S6 as markers of the PI3K/AKT pathway, and cleaved PARP as a marker of apoptosis.

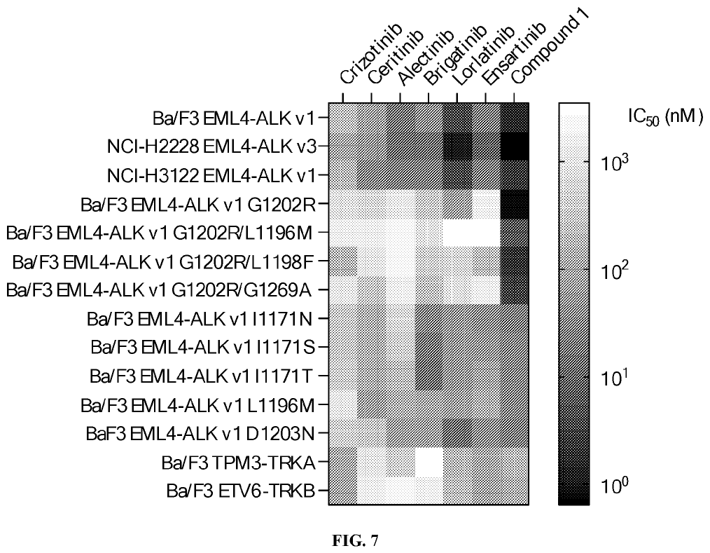

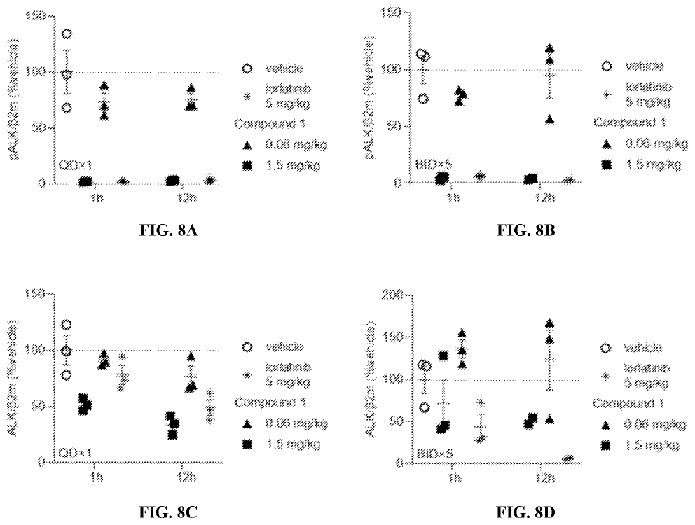

Results. Compound 1 dose-dependently reduced pALK levels upon QD×1 day treatment at the 1- and 12-hour time points, with a robust reduction observed at the dose of 1.5 mg/kg (a dose that induced regression, also referred to as a “regression dose”, data not shown). Lorlatinib 5 mg/kg QD×1 day reduced levels of pALK after 1 and 12 hours of treatment. Over this time period, 1.5 mg/kg Compound 1 and 5 mg/kg lorlatinib moderately reduced total ALK levels (FIG. 8A and FIG. 8C). Repeat dosing (BID×5 days) of Compound 1 and lorlatinib provided similar results, with Compound 1 dose-dependently reducing pALK levels and lorlatinib reducing pALK levels. Over the same time period, Compound 1 (1.5 mg/kg) and lorlatinib (5 mg/kg) moderately reduced total ALK levels, although ALK levels in vehicle-treated tumors were variable (FIG. 8B and FIG. 8D). The reductions in pALK are consistent with direct inhibition of ALK kinase activity by Compound 1 and lorlatinib.

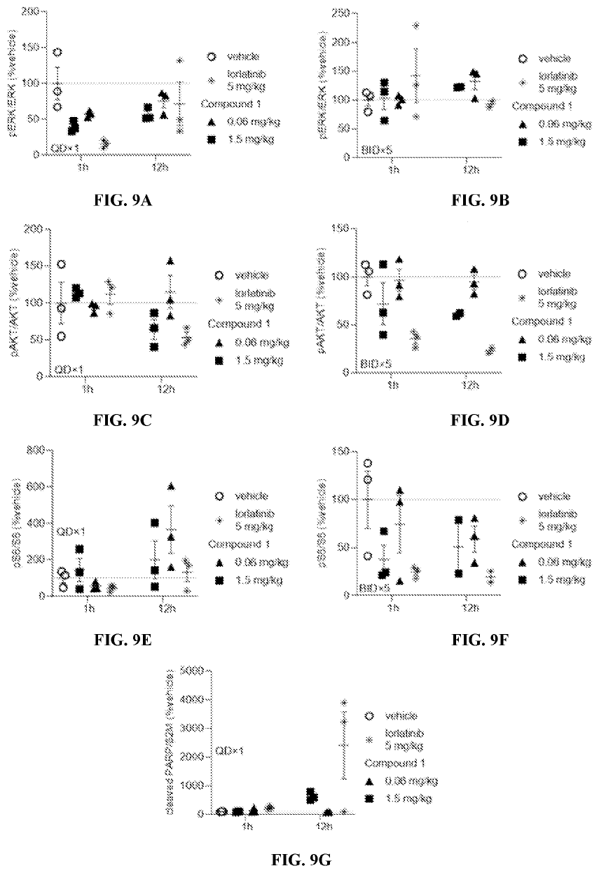

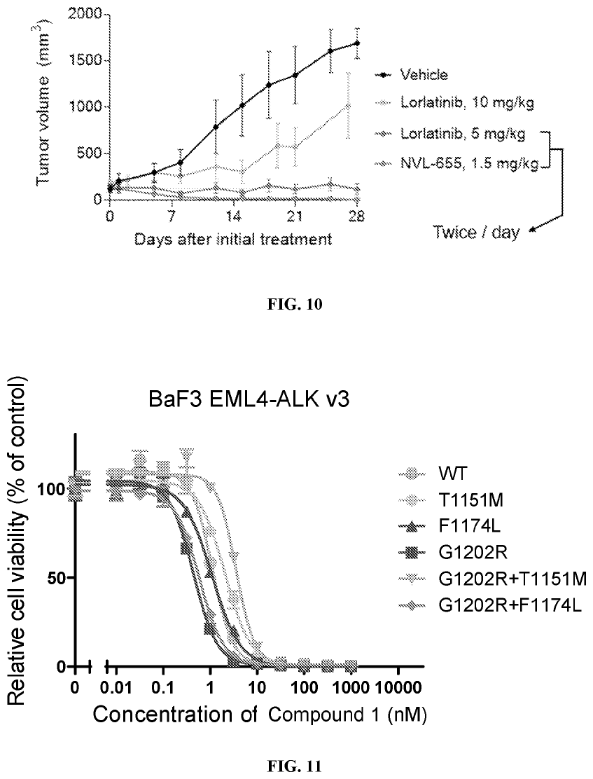

To evaluate the effect of treatment with Compound 1 on downstream signaling pathways, levels of pERK, a phospho-marker of the MAPK pathway, and pAKT and pS6, phospho-markers of the PI3K/AKT pathway, were monitored. QD×1 day Compound 1 dose-dependently reduced pERK levels, with lower levels observed at 1 hour compared with 12 hours post-treatment. QD×1 day lorlatinib 5 mg/kg behaved similarly (FIG. 9A). By contrast, Compound 1 and lorlatinib reduced levels of pAKT/AKT 12 hours but not 1 hour after QD×1 day treatment, albeit with a variable response in the vehicle group (FIG. 9C). For the pS6/S6 readout, lorlatinib modestly reduced pS6/S6 levels at 1 but not 12 hours after QD×1 day treatment, whereas Compound 1 did not dose-dependently reduce pS6/S6 levels (FIG. 9E).

The observation of tumor regression with Compound 1 prompted evaluation of a marker of apoptosis, cleaved PARP. Treatment with a single dose of Compound 1 (1.5 mg/kg) markedly increased levels of cleaved PARP at the 12-hour time point. This was not observed with Compound 1 at 0.06 mg/kg, a dose that did not induce regression. Treatment with a single dose of lorlatinib 5 mg/kg also increased cleaved PARP at the 12-hour time point (FIG. 9G).

The effect of repeated dosing (BID×5 days) of Compound 1 and lorlatinib on signaling through the MAPK and AKT pathways was also evaluated. Repeat dosing with Compound 1 or lorlatinib did not result in a continued reduction in pERK levels (FIG. 9B). By contrast, levels of pAKT/AKT and pS6/S6 were reduced upon repeat dosing (FIG. 9D and FIG. 9F).

### Example 10: Biochemical Assay

### Generation of Ba F3 Stable Cell Lines

Genes encoding CLIP1-LTK were synthesized, cloned into the retroviral construct pMSCV-puro (Biovector), and packaged into retroviral particles. The virus was used to infect Ba/F3 cells (RIKEN) at multiplicity of infection=1 or 10 for 1 day. Infected cells were rescued in media (RPMI-1640 with 10% fetal bovine serum and 1% streptomycin and penicillin) supplemented with mouse IL-3 (10 ng/mL) for 2 days, and stable cell lines were selected by IL-3 withdrawal and puromycin (0.8 μg/mL) for 7 days. Transformation of desired genes was confirmed by Sanger sequencing and western blot.

### Cell Proliferation Assay

Stable cells were plated at 1,000 cells/well (40 μL) in a 384-well plate for 1 day. Test compounds (40 nL) were then added in a 3-fold dilution series using the TECAN EVO200 liquid handler and incubated for 72 hours. Plates were equilibrated at room temperature for 15 minutes followed by addition of 40 μL CellTiter-Glo reagent (Promega). Luminescence was measured on a plate reader. Half-maximal inhibitory concentration (IC₅₀) was calculated from percent inhibition and inhibitor concentration using four-parameter logistic regression. The IC₅₀ data of several tested compounds are shown in the following table.

| Compound | Ba/F3 CLIP1-LTK cell viability IC₅₀ (nM) |
|---|---|
| Compound 1 | 2.7 |
| Alectinib | >971 |
| Brigatinib | 65 |
| Ceritinib | 219 |
| Crizotinib | 96 |
| Entrectinib | 164 |

### Example 11: a Phase 1, Open-Label Study the Food Effect, Potential Impact of pH Modification, and Drug-Drug Interaction Potential of Compound 1 in Healthy Subjects

Part A is a Phase 1, open-label, crossover study to investigate the effect of food and co-administration of lansoprazole on the PK of Compound 1 in healthy male and female subjects. Fourteen subjects are enrolled in Part A of the study.

Part B is a Phase 1, open-label, fixed sequence study to investigate the effect of multiple doses of Compound 1 on a single oral dose of midazolam in healthy male and female subjects. Fourteen subjects will be enrolled in this part of the study.

In CYP reaction phenotyping study performed in human liver microsomes (HLM) and in human recombinant CYP enzyme assays, Compound 1 was a substrate of CYP3A4 with potential contributions from CYP2C8 and CYP2C9. Therefore, the possibility exists for drug-drug interactions (DDIs) between Compound 1 and strong CYP3A4 inhibitors. In a study conducted in HLM with midazolam, Compound 1 was considered a moderate inhibitor of CYP3A4. Given the theoretical potential for Compound 1 to inhibit CYP3A4 mediated metabolism, a clinical drug interaction study is warranted to determine the effect of Compound 1 on the PK of midazolam, a commonly used substrate to evaluate CYP3A4 inhibition.

### Objectives:

Part A: Primary Objectives: To determine the effect of food on the single oral dose pharmacokinetics of Compound 1 in healthy subjects; to determine the impact of multiple oral doses of proton pump inhibitor (PPI) lansoprazole (perpetrator molecule), on the single oral dose pharmacokinetics of Compound 1 (victim molecule) in healthy subjects. Secondary Objective: to assess the safety and tolerability of Compound 1 when coadministered with lansoprazole in healthy subjects.
Part B: Primary Objective: to determine the impact of multiple oral doses of Compound 1 (perpetrator molecule) on the single oral dose pharmacokinetics of midazolam (victim molecule) in healthy subjects. Secondary Objective: to assess the safety and tolerability of Compound 1 when coadministered with midazolam in healthy subjects.

### Endpoints:

Part A: Primary Endpoint: primary pharmacokinetic parameters of Compound 1 determined using non-compartmental analysis: area under the concentration-time curve from time 0 extrapolated to infinity (AUC_{0-∞}), area under the concentration-time curve from time 0 to the time of the last quantifiable concentration (AUC₀₋ₜₗₐₛₜ), area under the concentration-time curve from time 0 to 24 hours (AUC₀₋₂₄), apparent terminal elimination half-life (t_{1/2}), apparent total clearance (CL/F), time of the maximum observed concentration (tₘₐₓ), and maximum observed concentration (Cₘₐₓ).
Part B: Primary Endpoint: primary pharmacokinetic parameters of midazolam determined using non-compartmental analysis: area under the concentration-time curve from time 0 extrapolated to infinity (AUC_{0-∞}), area under the concentration-time curve from time 0 to the time of the last quantifiable concentration (AUC₀₋ₜₗₐₛₜ), area under the concentration-time curve from time 0 to 24 hours (AUC₀₋₂₄), apparent terminal elimination half-life (t_{1/2}), apparent total clearance (CL/F), time of the maximum observed concentration (tₘₐₓ), and maximum observed concentration (Cₘₐₓ).

For Parts A & B: Secondary Endpoints: Incidence and severity of adverse events (AEs); incidence of laboratory abnormalities based on hematology, clinical chemistry, and urinalysis test results; 12-lead electrocardiogram (ECG) parameters; vital signs measurements; and physical examinations.

### Study Design:

### Part A:

In the food effect portion of the study, 14 subjects receive single oral doses of Compound 1 (15 mg) in either fed or fasted state. Half of the subjects (n=7) are randomized to receive Compound 1 under fed (high fat breakfast) conditions on Day 1 then receive Compound 1 under fasted conditions on Day 12, and half (n=7) are randomized to receive Compound 1 under fasted conditions on Day 1 then receive Compound 1 under fed conditions on Day 12.

In summary, 14 subjects receive the following treatment during the food effect portion of the study:
Day 1: single oral dose of 15 mg Compound 1 is administered either after an overnight fast (at least 10 hours) or 30 minutes after starting a standard high-fat breakfast.
Day 12: single oral dose of 15 mg Compound 1 is administered either after an overnight fast (at least 10 hours) or 30 minutes after starting a standard high-fat breakfast.

In the drug interaction portion of the study, 14 subjects receive the following treatments:
Day 24-29: once daily (QD) oral dose of 30 mg lansoprazole is administered after an overnight fast (at least 10 hours)
Day 30:30 mg lansoprazole coadministered with single oral dose of 15 mg Compound 1 after an overnight fast (at least 10 hours)

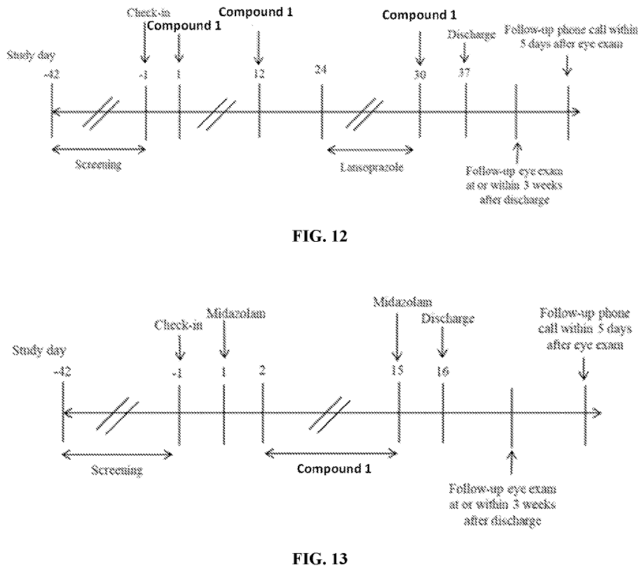

An overview of the study design is shown in FIG. 12.

Potential subjects are screened to assess their eligibility to enter the study within 42 days prior to the first dose administration. Subjects are admitted into the study site on Day 1 and be confined to the study site until discharge on Day 37. Subjects have an eye and optical coherence tomography (OCT) examination at or within 3 weeks after discharge, and a phone call follow-up within 5 days after the eye exam.

The total duration of study participation for each subject (from screening through phone follow up) is anticipated to be up to approximately 15 weeks.

### Part B:

### Fourteen Subjects Receive Each of the Following Treatments:

Day 1: single oral dose of 2 mg midazolam is administered after an overnight fast (at least 10 hours)
Days 2 to 14: oral doses of 15 mg Compound 1 QD is administered after an overnight fast (at least 10 hours)
Day 15: oral dose of 15 mg Compound 1 is coadministered with a single oral dose of 2 mg midazolam after an overnight fast (at least 10 hours)

An overview of the study design is shown in FIG. 13.

Potential subjects are screened to assess their eligibility to enter the study within 42 days prior to the first dose administration. Subjects are admitted into the study site on Day-1 and be confined to the study site until discharge on Day 16. Subjects have an eye and OCT examination at or within 3 weeks after discharge, and a phone call follow-up within 5 days after the eye exam.

The total duration of study participation for each subject (from screening through follow-up visit) is anticipated to be up to approximately 12 weeks.

Inclusion Criteria: Patients must meet all of the following criteria to be eligible to enroll in the study:
1. Males or females, of any race, between 18 and 65 years of age, inclusive.
2. Body mass index between 18.0 and 32.0 kg/m2, inclusive.
3. Estimated creatinine clearance ≥90 mL/min per Cockcroft-Gault formula at screening and check-in.
4. Aspartate aminotransferase, alanine aminotransferase and total bilirubin within normal limits at screening and check-in.
5. QTcF≤450 msec for men, ≤470 msec for women at screening and check-in.
6. In good health, determined by no clinically significant findings from medical history, 12-lead electrocardiogram (ECG), clinical laboratory evaluations (congenital nonhemolytic hyperbilirubinemia [eg, suspicion of Gilbert's syndrome based on total and direct bilirubin] is not acceptable) at screening and check-in, from vital signs measurements, eye and OCT examination during the screening period, and from the physical examination at screening or check-in, as assessed by the investigator (or designee).
7. Females will not be pregnant or lactating, and females of childbearing potential and males will agree to use contraception.
8. Able to comprehend and willing to sign an ICF and to abide by the study restrictions.

Exclusion Criteria: Patients meeting any of the following criteria are excluded from the study:
1. Medical Conditions
   a. Significant history or clinical manifestation of any metabolic, allergic, dermatological, hepatic, renal, hematological, pulmonary, cardiovascular, gastrointestinal, neurological, respiratory, endocrine, or psychiatric disorder, as determined by the investigator (or designee).
   b. History of significant hypersensitivity, intolerance, or allergy to any drug compound, food, or other substance, as determined by the investigator (or designee).
   c. History of stomach or intestinal surgery or resection that would potentially alter absorption or excretion of orally administered drugs (uncomplicated appendectomy and hernia repair will be allowed).
   d. Confirmed systolic blood pressure >140 or <90 mmHg, diastolic blood pressure >90 or <50 mmHg, and pulse rate >100 or <40 beats per minute (may repeat for confirmation if out of range per Investigator discretion).
   e. Positive hepatitis panel or positive human immunodeficiency virus test. Subjects whose results are compatible with prior immunization may be included at the discretion of the investigator.
2. Prior Concomitant Therapy
   a. Administration of a coronavirus disease 2019 (COVID-19) vaccine in the past 30 days prior to dosing.
   b. Use or intend to use any medications or products known to alter drug absorption, metabolism, or elimination processes, including St. John's wort, within 30 days prior to dosing, considered to potentially impact subject safety or the objectives of the study, as determined by the investigator (or designee).
   c. Use or intend to use any prescription medications or products other than hormone replacement therapy, oral, implantable, transdermal, injectable, or intrauterine contraceptives within 14 days prior to dosing, considered to potentially impact subject safety or the objectives of the study, as determined by the investigator (or designee).
   d. Use or intend to use slow release medications or products considered to still be active within 14 days prior to check in, considered to potentially impact subject safety or the objectives of the study, as determined by the investigator (or designee).
   e. Use or intend to use any nonprescription medications or products including vitamins, minerals, and phytotherapeutic, herbal, or plant derived preparations within 7 days prior to check in, considered to potentially impact subject safety or the objectives of the study, as determined by the investigator (or designee).
3. Prior/Concurrent Clinical Study Experience
   a. Dosing in a clinical study with an investigational drug (new chemical entity) in the past 30 days or 5 half-lives of that drug prior to dosing, whichever is longer.
   b. Have previously completed or withdrawn from this study or any other study investigating Compound 1, and have previously received Compound 1.
4. Diet and Lifestyle
   a. Alcohol consumption of >21 units per week for males and >14 units for females. One unit of alcohol equals 12 oz (360 mL) beer, 1.5 oz (45 mL) liquor, or 5 oz (150 mL) wine.
   b. Positive urine drug screen at screening or check-in or positive alcohol test result at check in.
   c. History of alcoholism or drug or chemical abuse within 2 years prior to check in.
   d. Use of tobacco or nicotine containing products within 3 months prior to check in, or positive cotinine at screening or check-in.
   e. Ingestion of Seville orange, or grapefruit containing foods or beverages within 7 days prior to check-in. Ingestion of poppy seed containing foods or beverages within 3 days prior to check-in.
5. Other
   a. Receipt of blood products within 2 months prior to check-in.
   b. Donation of blood from 3 months prior to screening, plasma from 2 weeks prior to screening, or platelets from 6 weeks prior to screening.
   c. Poor peripheral venous access.
   d. Subjects who, in the opinion of the investigator (or designee), should not participate in this study.

### Study Drug, Dosage, and Route of Administration

Compound 1 is supplied as tablets in 5 mg strength for oral administration. Lansoprazole is supplied as a capsules in 30 mg strength for oral administration. Midazoam is supplied as a solution at a concentration of 2 mg/mL.

Each dose of Compound 1, lansoprazole, and midazolam is administered orally with approximately 240 mL of room temperature water. When Compound 1 and lansoprazole, or Compound 1 and midazolam are administered concurrently, only 240 mL of room temperature water is administered for both drugs.

Subjects refrain from consuming water for 1 hour prior to dosing until 1 hour postdose, excluding the amount of water consumed at dosing and with breakfast (for food effect portion). At all other times during the study, subjects may consume water ad libitum. Subjects fast until after collection of the 4-hour PK sample collection on PK assessment days and 2 hours after drug administration on all other days. A standardized meal is provided after the 4-hour postdose blood sample collection.

Subjects are dosed seated and are not be permitted to lie supine for 2 hours after dosing, except as necessitated by the occurrence of an AE(s) or study procedures.

### Prior and Concomitant Medications and Therapies

Subjects refrain from use of any prescription or nonprescription medications or products during the study until the follow-up visit, unless the investigator (or designee), or sponsor have given their prior consent.

Paracetamol or acetaminophen (up to 2 g/day for up to 3 consecutive days), hormone replacement therapy, oral, implantable, transdermal, injectable, or intrauterine contraceptives are acceptable concomitant medications. The administration of any other concomitant medications during the study is prohibited without prior approval of the investigator (or designee), unless its use is deemed necessary for treatment of an AE. Any medication taken by a subject during the course of the study and the reason for its use will be documented in the source data.

### Example 12: Preparation of Tablets of Compound 1

Compound 1 tablets were manufactured for oral administration at 5 mg, 25 mg, 50 mg, 75 mg, 100 mg, 125 mg, and 150 mg strengths. Tablets at all dosage strengths were manufactured as immediate release, film-coated tablets. The quantitative composition of the tablets is provided in the following table.

TABLE 2
Composition of Compound 1 Tablets, 5 mg, 25
mg, 50 mg, 75 mg, 100 mg, 125 mg, and 150 mg

| | | Target Quantity (mg/tablet) | | | | | | |
|---|---|---|---|---|---|---|---|---|
| Component | Function | 5 mg | 25 mg | 50 mg | 75 mg | 100 mg | 125 mg | 150 mg |
| Compound 1 | Active | 5.00 | 25.00 | 50.00 | 75.00 | 100.00 | 125.00 | 150.00 |
| Drug | | | | | | | | |
| Substance^{a} | | | | | | | | |
| Microcrystalline | Diluent | 83.50 | 85.00 | 170.00 | 255.0 | 340.00 | 425.00 | 510.00 |
| Cellulose^{a} | | | | | | | | |
| Croscarmellose | Disintegrant | 5.00 | 6.25 | 12.50 | 18.75 | 25.00 | 31.25 | 37.50 |
| Sodium | | | | | | | | |
| Colloidal Silica | Glidant | 2.50 | 3.125 | 6.25 | 9.375 | 12.50 | 15.625 | 18.75 |
| Dioxide | | | | | | | | |
| Hydroxypropyl | Binder | 2.50 | 3.125 | 6.25 | 9.375 | 12.50 | 15.625 | 18.75 |
| Cellulose | | | | | | | | |
| Magnesium | Lubricant | 1.50 | 2.50 | 5.00 | 7.50 | 10.00 | 12.5 | 15.00 |
| Stearate | | | | | | | | |
| Opadry II ^{b} | Cosmetic | 3.00 | 3.125 | 6.25 | 9.375 | 12.50 | 15.625 | 18.75 |
| | film-coat | | | | | | | |
| Purified Water^{c} | Coating | qs | qs | qs | qs | qs | qs | qs |
| | Agent | | | | | | | |

| | | | | | | | |
|---|---|---|---|---|---|---|---|
| Total | 103.00 | 128.125 | 256.25 | 384.375 | 512.5 | 640.625 | 768.75 |

^{a}The amount of drug substance and microcrystalline cellulose may be adjusted depending on the potency of the drug substance
^{b} Non-functional, cosmetic film coating, added to target theoretical weight gain of 2.5-3%.
^{c}Removed during processing.
qs: quantity sufficient

### Example 13: Biochemical and Cell Assay to Test Mutations

Biochemical Kinase Assay: Recombinant ALK kinase domains containing the wild-type sequence or containing one or more amino acid substitutions are generated. The inhibitory activity of compounds against recombinant ALK kinase domains is measured using a biochemical in vitro phosphorylation assay. Kinase reaction is initiated by mixing test compounds (dilution series ranging from 10 μM to 0 nM), ATP (1 mM), the fluorogenic phosphorylation substrate AQT0101 (15 μM, AssayQuant), and ALK kinase domains in buffer (54 mM HEPES pH 7.5, 0.012% Brij-35, 0.52 mM EGTA, 1.2 mM DTT, 1% glycerol, 0.2 mg/mL BSA, 10 mM MgCl₂). The plate is sealed and read by SpectraMax Paradigm at λ=485 nm every 2 minutes for 120 minutes at 30° C. Initial rates of reaction (v) are calculated from the change in fluorescence intensity over time during the initial, linear portion of the reaction. Half-maximal inhibitory concentration (IC₅₀) is determined from regression of v and log [I] (with “I” denoting inhibitor concentration) to the four-parameter logistic equation. Alternatively, apparent inhibitory constant (Kᵢ^{app}) is determined from regression of v and I to Morrison Equation (“E” denotes enzyme concentration):

$$v = 1 - \frac{( E + I + {K}_{i}^{a p p} ) - \sqrt{{( E + I + {K}_{i}^{a p p} )}^{2} - 4 EI}}{2 E}$$

Generation of Ba/F3 Stable Cell Lines: Genes encoding EML4-ALK v1 containing the wild-type sequence or containing one or more point mutations are synthesized, cloned into a retroviral construct, and packaged into retroviral particles. The virus is used to infect Ba/F3 cells (RIKEN) at multiplicity of infection in the range of 1 to 10 for 1 day. Infected cells are rescued in media (RPMI-1640 with 10% fetal bovine serum and 1% streptomycin and penicillin) supplemented with mouse IL-3 (10 ng/mL) for 2 days, and stable cell lines are selected by IL-3 withdrawal and puromycin (0.8 μg/mL) for 7 days. Transformation of the desired gene is confirmed by Sanger sequencing and western blot using the ALK antibody.

Cell Proliferation Assay: Stable cells are plated at 1,000 cells/well (40 μL) in a 384-well plate for 1 day. Test compounds (40 nL) are then added in a 3-fold dilution series using the TECAN EVO200 liquid handler and incubated for 72 hours. Plates are equilibrated at room temperature for 15 minutes followed by addition of 40 μL CellTiter-Glo reagent (Promega). Luminescence is measured on a plate reader. Half-maximal inhibitory concentration (IC₅₀) is calculated from percent inhibition and inhibitor concentration using four-parameter logistic regression.

### Example 14: Evaluation of ctDNA

Method: Whole blood samples are collected at clinical trial sites in STRECK cell-free DNA BCT® tubes and shipped at ambient temperature to central lab for plasma generation and storage at −80° C. Afterwards, the plasma samples are analyzed to determine the genomic alternations in the circulating tumor DNA (ctDNA) via a hybrid capture based next generation sequencing method.

Results: The ctDNA analysis is conducted to evaluate the impact of Compound 1 on ALK variant allele frequencies (VAF), including both ALK fusion alleles and ALK mutant alleles. The correlation between the dose levels of Compound 1 and the suppression on ALK VAFs are evaluated. These data demonstrate the activity of Compound 1 on ALK fusion and mutations and help support the on-target activity of the compound in patients.

### Example 15: Phase 1/2 Clinical Study

This example is an updated version of Example 1. A FIH, Phase 1/2, multicenter, open-label, dose escalation and expansion study is conducted to evaluate the safety and tolerability of Compound 1, to determine the RP2D, and, if applicable, the MTD, and to evaluate the antitumor activity in patients with advanced ALK-positive NSCLC and other advanced ALK-positive solid tumors.

### Phase 1 Objectives:

Primary Objective: to determine the RP2D and/or maximum tolerated dose (MTD) of Compound 1 in patients with advanced ALK-positive solid tumors.

Secondary Objectives: to evaluate the overall safety and tolerability of Compound 1; to characterize the PK profile of Compound 1; to evaluate preliminary antitumor activity of Compound 1 in patients with advanced ALK positive solid tumors.

Exploratory Objectives: to evaluate additional tumor and blood-based molecular markers of response and resistance to Compound 1; to evaluate the metabolite profile of Compound 1; to evaluate extracranial activity.

### Phase 2 Objectives:

Primary Objective: to evaluate the efficacy of Compound 1 at the RP2D in patients with advanced ALK-positive NSCLC, including those with ALK resistance mutations, and other solid tumors.

Secondary Objectives: to assess additional measures of clinical efficacy in patients with ALK-positive NSCLC, including those with ALK resistance mutations, and other solid tumors; to evaluate the intracranial antitumor activity of Compound 1 at the RP2D in patients with advanced ALK-positive NSCLC and other solid tumors; to characterize the safety and tolerability of Compound 1 at the RP2D; to confirm the PK profile of Compound 1 at the RP2D.

Exploratory Objectives: to evaluate additional tumor and blood-based molecular markers of response and resistance to Compound 1; to assess progression-free survival on subsequent therapy; to evaluate extracranial activity.

### Phase 1 Endpoints:

Primary Endpoint: RP2D and, if applicable, the MTD as determined by incidence of dose-limiting toxicities (DLTs) during Cycle 1, overall safety profile, PK, PD, and preliminary efficacy

### Secondary Endpoints:

Incidence and severity of treatment-emergent adverse events (TEAEs) and changes in clinically relevant laboratory parameters
Pharmacokinetic parameters of Compound 1: Maximum plasma concentration (Cₘₐₓ); Cₘₐₓ—dose normalized, plasma concentration at the end of the dosing interval (Cₜₐᵤ); average plasma concentration (C_{avg}); time of maximum concentration (Tₘₐₓ); area under the curve at the end of the dosing interval (AUCₜₐᵤ); AUCₜₐᵤ—dose normalized, area under the curve from time 0 to 24 (AUC₀₋₂₄); AUC₀₋₂₄—dose normalized, area under the curve from time 0 to infinity (AUC_{inf}); AUC_{inf}—dose normalized; oral clearance (CL/F); volume of distribution (V_{z}/F); and half-life (t_{1/2})
Objective response rate (ORR) per Response Evaluation Criteria in Solid Tumors Version 1.1 (RECIST 1.1)—Defined as the percent of patients with a complete response (CR) or partial response (PR) according to RECIST 1.1.
Duration of response (DOR) per RECIST 1.1—In responders, defined as the time from first response per RECIST 1.1 to radiographic disease progression or death
Intracranial ORR (IC-ORR) per RECIST 1.1—In patients with measurable metastatic CNS disease up to 5 intracranial target lesions are accessed for response, according to RECIST 1.1
Intracranial DOR (IC-DOR) per RECIST 1.1—In patients with intracranial response, defined as the time from first IC-response per RECIST 1.1 to radiographic IC-disease progression or death
Clinical benefit rate (CBR) per RECIST 1.1—Defined as the percent of patients with a confirmed CR or PR, or stable disease (SD) of at least 24 weeks duration according to RECIST 1.1
Time to response per RECIST 1.1—Defined as the time from first dose to first confirmed radiographic response according to RECIST 1.1
Progression-free survival (PFS) per RECIST 1.1—Defined as the time from first dose to radiographic disease progression per RECIST 1.1 or death

### Exploratory Endpoints:

Baseline levels and/or post-treatment changes in tumor and blood biomarker levels including but not limited to expression of proteins, phospho-proteins, genes, variant allele frequency, and genetic variations
Compound 1 metabolite identification and quantification in post-treatment blood samples
Extracranial-ORR: In patients with extracranial metastatic disease, defined as the percent of patients with an extracranial response according to RECIST 1.1 per Investigator assessment

### Phase 2 Endpoints:

Primary Endpoint: ORR per RECIST 1.1—Defined as the proportion of patients with a confirmed CR or PR according to RECIST 1.1 per Blinded Independent Central Review (BICR) of the patients confirmed to have measurable disease at baseline per BICR.

### Secondary Endpoints

DOR per RECIST 1.1—In responders, defined as the time from first BICR-assessed confirmed response per RECIST 1.1 to radiographic disease progression or death
CBR per RECIST 1.1—Defined as the percent of patients with a confirmed CR or PR, or SD of at least 24 weeks duration according to RECIST 1.1 per BICR
Time to response per RECIST 1.1—Defined as the time from first dose to first confirmed radiographic response according to RECIST 1.1 per BICR
PFS per RECIST 1.1—Defined as the time from first dose to radiographic disease progression per RECIST 1.1 based on BICR assessment or death
Overall survival (OS)—Defined as the time from first dose to death due to any cause
IC-ORR per RECIST 1.1—In patients with measurable metastatic CNS disease up to 5 intracranial target lesions are assessed for response according to RECIST 1.1 per BICR of the patients confirmed to have measurable intracranial disease at baseline per BICR
IC-DOR per RECIST 1.1—In patients with intracranial response, defined as the time from first BICR-assessed confirmed response per RECIST 1.1 to radiographic disease progression or death.
Time to IC-response per RECIST 1.1—In patients with metastatic CNS disease, defined as the time from first dose to first confirmed radiographic IC-response according to RECIST 1.1 per BICR
Intracranial PFS (IC-PFS) per RECIST 1.1—In patients with metastatic CNS disease, defined as the time from first dose to radiographic disease progression per RECIST 1.1 based on BICR assessment or death
Incidence and severity of TEAEs and changes in clinically relevant laboratory parameters
Pharmacokinetic parameters of Compound 1—Cₘₐₓ, Cₘₐₓ—dose normalized, Cₜₐᵤ, C_{avg}, Tₘₐₓ, AUCₜₐᵤ, AUCₜₐᵤ—dose normalized, AUC₀₋₂₄, AUC₀₋₂₄—dose normalized, AUC_{inf}, AUC_{inf}—dose normalized, CL/F, V_{z}/F, t_{1/2}

### Exploratory Endpoints:

Baseline levels and/or post-treatment changes in tumor and blood biomarker levels including but not limited to expression of proteins, phospho-proteins, genes, variant allele frequency, and genetic variations
PFS2: Defined as the time from beginning of treatment with Compound 1 until disease progression on subsequent therapy (based on local assessment or medical record report of progression on subsequent therapy) or death due to any cause
EC-ORR: In patients with extracranial metastatic disease, defined as the percent of patients with an extracranial response according to RECIST 1.1 per Investigator assessment

Study Design: The study is conducted in 2 phases (FIG. 1).

The Phase 1 dose-escalation includes patients with locally advanced or metastatic solid tumors harboring an ALK rearrangement or activating ALK mutation. Patients with ALK fusion-positive NSCLC must have previously received ≥1 ALK TKI, one of which must be a 2nd or 3^{rd} generation TKI (ceritinib, alectinib, brigatinib, or lorlatinib), while those with other solid tumors must have previously received ≥1 prior systemic anticancer therapy or be those for whom no satisfactory standard therapy exists. The Phase 1 portion of the study is designed to determine the RP2D and, if applicable, the MTD and to assess the safety of Compound 1.

The Phase 1 portion of the study employs a BOIN design with a 3+3 run-in to evaluate dose escalation. The DLT observation period is defined from first dose on CID1 through to the end of the first cycle (21-day cycle period). After each group of patients are treated at a given dose level and monitored for 21 days, available data (to include, but not limited to, the incidence of DLT/other AEs and preliminary PK) are reviewed. Initiation of the next dosing group depends on the occurrence of DLT, outcome of the BOIN analysis, and the recommendation with consideration to the overall safety profile. The BOIN dose-escalation is declared complete when the number of evaluable patients treated at the current dose reaches 12 and the decision is to stay at the current dose, or when the maximum sample size is reached. However, if an optimal biologic dose or maximal feasible dose is reached prior to the completion of the BOIN dose-escalation, the RP2D may be declared. Patients with a DLT are not replaced. Patients who are unevaluable for DLT (do not receive ≥16 days of intended dosing and/or do not complete the 21-day observation period for reasons other than DLT during the first cycle) may be replaced.

After a DLT has occurred, if continued treatment with Compound 1 is determined to be in the patient's best interest, treatment with Compound 1 may resume at the same or a reduced dose.

During dose escalation, when available data support the safety, PK, and clinical activity of a given dose level, additional patients (up to a total of 12 patients, including patients already included in BOIN dose escalation) may be enrolled at that dose level. These patients are monitored for DLT and included in the evaluation for the conduct of the BOIN dose-escalation. A total of up to approximately 54 patients may be treated in the Phase 1 dose-escalation.

In order to further evaluate safety and efficacy and to confirm the RP2D, up to 20 additional patients may be treated at the RP2D, or at each of one or more candidate RP2Ds, prior to initiation of the Phase 2 portion of the study.

The selection of the RP2D occurs after a sufficient number of patients have enrolled and completed the first imaging/efficacy assessment and the data have been reviewed. The RP2D is expected to be an MTD or a lower optimal biological dose, or a maximum feasible dose, and is the dose level deemed as suitable for testing in the Phase 2 portion of the study. The selection of the RP2D is made using the totality of the data obtained from Phase 1, and includes the evaluation of clinical PK, pharmacodynamics, safety, and antitumor activity.

### Phase 2 Includes 4 Cohorts:

Cohort 2a: Patients with locally advanced or metastatic NSCLC harboring an ALK rearrangement who have received 1 prior 2nd-generation ALK TKI (ceritinib, alectinib, or brigatinib).
Cohort 2b: Patients with locally advanced or metastatic NSCLC harboring an ALK rearrangement, who have received 2-3 prior 1^{st} or 2^{nd} generation ALK TKIs (crizotinib, ceritinib, alectinib, or brigatinib)
Cohort 2c: Patients with locally advanced or metastatic NSCLC harboring an ALK rearrangement, who have received 2-3 prior 1^{st} or 2^{nd}-generation ALK TKIs (crizotinib, ceritinib, alectinib, or brigatinib), with lorlatinib in the 2^{nd} or 3^{rd} line.
Cohort 2d: Patients with other solid tumors harboring an ALK rearrangement or activating ALK mutation, including patients with NSCLC not eligible for cohorts 2a-c, who have received ≥1 prior systemic anticancer therapy, or for whom no satisfactory standard therapy exists.

The Phase 2 portion of the study is designed to estimate the ORR and the corresponding 95% CI for the following populations of patients: all treated patients with ALK-positive NSCLC pooled across cohorts 2a, 2b and 2c; each individual cohort 2a, 2b and 2c; and patients with ALK resistance mutations of interest (i.e., any ALK resistance mutation; the ALK G1202R mutation, whether occurring with or without other ALK mutations) pooled across cohorts 2a, 2b and 2c.

Phase 1 Dose Levels: Phase 1 evaluates ascending doses of Compound 1 monotherapy administered orally once daily (QD) continuously until disease progression, unacceptable toxicity, or voluntary withdrawal of consent. Under the BOIN design, up to approximately 54 patients are enrolled and treated at the dose levels presented in the following table.

| Dose level | Compound 1 Dose | | Frequency* |
|---|---|---|---|
| DL1 | 15 | mg | QD |
| DL2 | 25 | mg | QD |
| DL3 | 50 | mg | QD |
| DL4 | 100 | mg | QD |
| DL5 | 150 | mg | QD |
| DL6** | 200 | mg | QD |

*BID dosing may be explored at any DL.
**Higher DLs may be explored.

Intermediate dose levels may be explored during dose escalation; a lower dose level (e.g., 10 mg QD) may be explored based on results of the BOIN.

Recommended Phase 2 Dose: The recommended Phase 2 dose (RP2D) is expected to be an MTD or a lower optimal biological dose, or a maximum feasible dose, and is the dose level deemed as suitable for testing in the Phase 2 portion of the study. The selection of the RP2D is made using the totality of the data obtained from Phase 1, and includes the evaluation of clinical PK, pharmacodynamics, safety, and antitumor activity.

Duration of Treatment: Across both phases, patients receive study drug continuously from first dose until independent radiological confirmation of disease progression, unacceptable toxicity, withdrawal by patient, termination the study, or commercial availability. Patients who are deriving clinical benefit may continue to receive Compound 1 following disease progression.

End of Study Definition: The end of the study is defined as the date of the last visit of the last patient in the study or termination of the study.

Inclusion Criteria: Patients must meet all of the following criteria to be eligible to enroll in the study:
1. Age ≥18 years. (Phase 2 Cohort 2d only: Age ≥12 years and weighing >40 kg.)
2. Disease criteria
   a. Phase 1: Histologically or cytologically confirmed locally advanced or metastatic solid tumor with a documented ALK rearrangement or activating ALK mutation detected by certified assay (i.e. CLIA in the US).
   b. Cohorts 2a, 2b, and 2c: Histologically or cytologically confirmed locally advanced or metastatic NSCLC (excluding patients with documented transformation to non-NSCLC histology) with a documented ALK rearrangement detected by certified assay (i.e. CLIA in the US).
   c. Cohort 2d: Any other histologically or cytologically confirmed locally advanced or metastatic solid tumor with a documented ALK rearrangement or activating ALK mutation detected by certified assay (i.e. CLIA in the US), including but not limited to inflammatory myofibroblastic tumors, esophageal squamous cell carcinoma, renal medullary carcinoma, renal cell carcinoma, breast cancer, colorectal cancer, ovarian cancer, papillary thyroid carcinoma, cholangiocarcinoma, spitzoid tumors, neuroblastoma, anaplastic thyroid cancer, and patients with NSCLC not eligible for Cohorts 2a-c.
3. Prior anticancer treatment
   a. Phase 1: Patients with ALK fusion-positive NSCLC must have previously received ≥1 ALK TKI, one of which must be a 2^{nd} or 3^{rd} generation TKI (ceritinib, alectinib, brigatinib, or lorlatinib). Patients with other solid tumors must have previously received ≥1 prior systemic anticancer therapy or be those for whom no satisfactory standard therapy exists.
   b. Cohort 2a: 1 prior 2^{nd} generation ALK TKI (ceritinib, alectinib, or brigatinib).
   c. Cohort 2b: 2-3 prior 1^{st} or 2^{nd} generation ALK TKIs (crizotinib, ceritinib, alectinib, or brigatinib).
   d. Cohort 2c: 2-3 prior ALK TKIs, with lorlatinib received in the 2^{nd} or 3^{rd} line of therapy.
   e. All patients except Phase 2 Cohort 2d: ≤2 prior lines of chemotherapy and/or immunotherapy in the locally advanced or metastatic setting. Patients who have received >2 prior lines of chemotherapy and/or immunotherapy in the locally advanced or metastatic setting may be enrolled in Phase 2 Cohort 2d.
   f. Phase 2 Cohorts 2a, 2b, and 2c: No prior investigational agents targeting ALK. Patients who previously received investigational agents targeting ALK may be enrolled in Phase 1 and Phase 2 Cohort 2d.
4. Phase 1: Must have evaluable disease (target or nontarget) according to RECIST 1.1. Phase 2: Must have measurable disease, defined as ≥1 radiologically measurable target lesion according to RECIST 1.1 (Oken et al., Am. J. Clin. Oncol. 1982, 5 (6): 649-655).
5. Pre-treatment tumor tissue (archived, if available, or a fresh biopsy) submitted for central analysis. It is preferable that submitted tumor tissue be obtained during or after the most recent disease progression. If appropriate tissue is not available, and if biopsy is not considered safe and medically feasible by the Investigator, the patient may be approved for enrollment after consultation with the Sponsor's Medical Monitor.
6. Eastern Cooperative Oncology Group (ECOG) performance status (PS) of 0, 1 or 2 (Eisenhauer et al., Eur. J. Cancer 2009, 45 (2): 228-247).
7. Adequate organ function and bone marrow reserve as indicated by the following laboratory assessments performed prior to the first dose of study drug:
   a. Bone marrow function: absolute neutrophil count (ANC)≥1500/μL; platelet count >75,000/μL; hemoglobin ≥8 g/dL.
   b. Renal function: estimated creatinine clearance ≥60 mL/min Hepatic function: bilirubin <1.5×ULN, unless evidence of Gilbert Syndrome, in which the patient must have total bilirubin ≤3.0 mg/dL; aspartate aminotransferase and alanine aminotransferase≤3.0×ULN (≤5.0×ULN if liver metastases involvement).
8. All clinically relevant toxicities related to prior anticancer therapy must have recovered to Grade ≤1 or baseline (except alopecia or ototoxicity).
9. Women of childbearing potential (WOCBP) and male patients must be surgically sterile or be willing to abstain from sexual activity or use a highly effective contraceptive method from the time of signing the ICF through at least 6 months after the last administration of study drug for women, and at least 4 months after the last administration of study drug for men (or longer, if required by local or country-specific guidance). “Highly effective contraceptive method” is defined consistent with World Health Organization (WHO) criteria in CTFG 2020, which is incorporated herein by reference.
10. Provide written informed consent and willing and able to comply with requirements of the study protocol. Assent must be obtained for patients <18 years old (Phase 2 Cohort 2d only) and a parent/guardian must provide written consent.

Exclusion Criteria: Patients meeting any of the following criteria are excluded from the study:
1. Patient's cancer has a known primary driver alteration other than ALK.
2. Known allergy/hypersensitivity to excipients of Compound 1.
3. Major surgery within 4 weeks of study entry. Minor surgical procedures (e.g., port insertion) are permitted, but with sufficient time for wound healing as deemed clinically appropriate.
4. Ongoing or recent anticancer therapy within the following timeframe prior to first dose of study drug (Compound 1 may be started within limits for prior TKI or chemotherapy if considered to be safe and within the best interest of the patient, with prior approval):
   a. TKI or other non-chemotherapy/non-immunotherapy anticancer agents <5 half-lives or <7 days, whichever is longer
   b. Chemotherapy <21 days
   c. Immunotherapy or cellular therapy <28 days
5. Ongoing or recent radiation therapy within the following timeframe prior to first dose of study drug:
   a. Radiation therapy (except palliative radiation to relieve bone pain)<14 days
   b. Palliative radiation to relieve bone pain <48 hours
   c. Stereotactic or small field brain irradiation <7 days
   d. Whole brain radiation <14 days
6. Prior high-dose chemotherapy requiring stem cell rescue.
7. Uncontrolled clinically relevant bacterial or fungal infection requiring systemic therapy.
8. Has known active tuberculosis or active Hepatitis B or C. Active Hepatitis B is defined as a known positive HBsAg result and known quantitative HBV DNA results greater than the lower limits of detection of the assay. Active Hepatitis C is defined by a known positive Hep C Ab result and known quantitative HCV RNA results greater than the lower limits of detection of the assay.
9. Patient has a QTcF consistently >450 msec. Patient has a history of prolonged QT syndrome or Torsades de pointes.
10. Patients with clinically significant cardiovascular disease as follows:
   a. Within 3 months of enrollment: cerebral vascular accident/stroke; myocardial infarction; unstable angina; uncontrolled atrial fibrillation of any grade
   b. History of congestive heart failure (New York Heart Association Classification Class ≥II); second-degree or third-degree atrioventricular block (unless paced) or any atrioventricular block with PR consistently >220 msec; or ongoing cardiac dysrhythmias of NCI-CTCAE Grade ≥2.
11. Patient has central nervous system (CNS) metastases or a primary CNS tumor that is associated with progressive neurological symptoms or requires increasing doses of corticosteroids to control the CNS disease. If a patient requires corticosteroids for management of CNS disease, the dose must have been stable for the 2 weeks preceding C1D1. Asymptomatic leptomeningeal carcinomatosis is allowed.
12. Symptomatic spinal cord compression.
13. Patients with moderate to severe cognitive impairment or psychiatric disturbances that would compromise the patient's ability to comply with study requirements.
14. Evidence of active malignancy (other than current ALK-positive solid malignancy) requiring systemic therapy within the prior 2 years.
   a. Exceptions: nonmelanoma skin cancer, in situ melanoma, in situ cervical cancer, papillary thyroid cancer, ductal carcinoma in situ of the breast, or localized and presumed cured prostate cancer. Patients on long-term anti-hormonal therapy for a prior malignancy are allowed as long as the malignancy has not been active within the prior 2 years.
15. Concomitant use (within 12 days of enrollment) of strong CYP3A4 inducers or strong CYP3A4 inhibitors.
16. Manifestation of malabsorption due to prior gastrointestinal surgery, disease, or other illness that could affect oral absorption, distribution, metabolism, or excretion of the study drug.
17. Patient is pregnant or breast-feeding. WOCBP must have a negative serum pregnancy test at Screening and negative serum or urine tests prior to first dose of study drug.
18. Actively receiving systemic treatment or direct medical intervention on another therapeutic clinical study.
19. Any evidence of current ILD or pneumonitis or a prior history of ILD or non-infectious pneumonitis.
20. Any medical condition or laboratory abnormality that would pose a risk to study patient or confound the ability to interpret study results.

Study Drug, Dosage, and Route of Administration: Compound 1 is supplied as tablets for oral administration in 2 strengths: 5 mg and 50 mg. Compound 1 should be taken on an empty stomach, at least 1 hour before and no sooner than 2 hours after ingestion of food and/or beverages other than water.

### Prior and Concomitant Medications and Therapies:

In certain embodiments, concomitant use (within 12 days of enrollment) of drugs that are known strong CYP3A4 inducers (such as phenobarbital, rifampin, phenytoin, carbamazepine, rifabutin, rifapentine, clevidipine, St. John's Wort) are prohibited. In certain embodiments, concomitant use (within 12 days of enrollment) of drugs that are known strong CYP3A4 inducers (such as phenobarbital, rifampin, phenytoin, carbamazepine, rifabutin, rifapentine, clevidipine, St. John's Wort) are permitted. In certain embodiments, concomitant use (within 12 days of enrollment) of drugs that are known strong CYP3A4 inducers (such as phenobarbital, rifampin, phenytoin, carbamazepine, rifabutin, rifapentine, clevidipine, St. John's Wort) are used with caution.

In certain embodiments, concomitant use (within 12 days of enrollment) of drugs that are known strong CYP3A4 inhibitors are prohibited. In certain embodiments, concomitant use (within 12 days of enrollment) of drugs that are known strong CYP3A4 inhibitors are permitted. In certain embodiments, concomitant use (within 12 days of enrollment) of drugs that are known strong CYP3A4 inhibitors are used with caution. In certain embodiments, the strong CYP3A4 inhibitors are clarithromycin, erythromycin, diltiazem, itraconazole, ketoconazole, ritonavir, verapamil, Goldenseala, and Grapefruitb (including juices).

In certain embodiments, concomitant use (within 12 days of enrollment) of drugs that are strong inhibitors of CYP3A4, sensitive substrates of CYP3A4 and/or CYP2C8, substrates of P-glycoprotein (P-gp)/multidrug resistance protein 1 (MDR1), substrates of BCRP/breast cancer resistance protein (ABCG2), substrates of OATP1B1, substrates of OATP1B3, or substrates of MATE1 are used with caution. In certain embodiments, concomitant use (within 12 days of enrollment) or co-administration of Compound 1 and any one or more drugs that are strong inhibitors of CYP3A4, sensitive substrates of CYP3A4 and/or CYP2C8, substrates of P-glycoprotein (P-gp)/multidrug resistance protein 1 (MDR1), substrates of BCRP/breast cancer resistance protein (ABCG2), substrates of OATP1B1, substrates of OATP1B3, or substrates of MATE1 are permitted. In certain embodiments, concomitant use (within 12 days of enrollment) or co-administration of drugs that are strong inhibitors of CYP3A4, sensitive substrates of CYP3A4 and/or CYP2C8, substrates of P-glycoprotein (P-gp)/multidrug resistance protein 1 (MDR1), substrates of BCRP/breast cancer resistance protein (ABCG2), substrates of OATP1B1, substrates of OATP1B3, or substrates of MATE1 are prohibited.

In certain embodiments, concomitant use (within 12 days of enrollment) or co-administration of gastric acid reducing agents with Compound 1 should be avoided. In certain embodiments, concomitant use (within 12 days of enrollment) or co-administration of gastric acid reducing agents with Compound 1 should be used with caution. In certain embodiments, concomitant use (within 12 days of enrollment) or co-administration of gastric acid reducing agents with Compound 1 is permitted.

### Exemplified Agents Provided Herein Include, but are not Limited to:

CYP3A4 Inducers: phenobarbital, rifampin, phenytoin, carbamazepine, rifabutin, rifapentine, clevidipine, St. John's Wort
CYP3A4 Inhibitors: clarithromycin, erythromycin, diltiazem, itraconazole, ketoconazole, ritonavir, verapamil, Goldenseal, Grapefruit (including juices)
CYP3A4 Substrates (sensitive): buspirone, everolimus, lovastatin, midazolam, simvastatin, triazolam, maraviroc, conivaptan, darifenacin
CYP2C8 Substrates (sensitive): repaglinide, montelukast, pioglitazone, rosiglitazone
P-gp Substrates: digoxin, fexofenadine, loperamide, quinidine, talinolol, vinblastine
BCRP Substrates: daidzein, dantrolene, estrone-3-sulfate, prazosin, sulfasalazine
OATP1B1/OATP1B3 Substrates: antiviral protease inhibitors (e.g. ritonavir), clarithromycin, cyclosporine, gemfibrozil, rifampin, simeprevir
MATE1 Substrates: metformin, tetra-ethylammonium (TEA), cimetidine, procainamide

The following medications and therapies are excluded during the study:
Anticancer therapies, including investigational therapy, or concurrent radiation therapy (except palliative radiotherapy as noted below)
   Palliative radiation during study treatment must be approved prior to implementation. Palliative radiation to a RECIST 1.1 target lesion prior to radiographic documentation of disease progression is strongly discouraged
   Hormonal therapy including octreotide or bone targeted therapy such as bisphosphonates or a receptor activator of nuclear factor-kappa B ligand inhibitor is not considered as anticancer therapy.
Any other investigational therapy
Medications that are strong inducers of CYP3A4

### Example 16: Preparation of Form 2 of Compound 1

Form 2 of Compound 1 was prepared as follows: to approximately 100 mg of amorphous Compound 1 in the 4 ml vial, 200 μl of 2-methyl-THF was added to form a clear solution. The sample was left uncapped to let the solvent evaporates overnight at room temperature and pressure. The resulting form was quickly dried by heating up to 200° C. under vacuum in a Buchi glass oven piston.

### Example 17: Combination Study with Osimertinib

To demonstrate the potential benefits of combination therapies, Compound 1 is tested in MR240 models in combination with an EGFR inhibitor osimertinib in the following four studies.

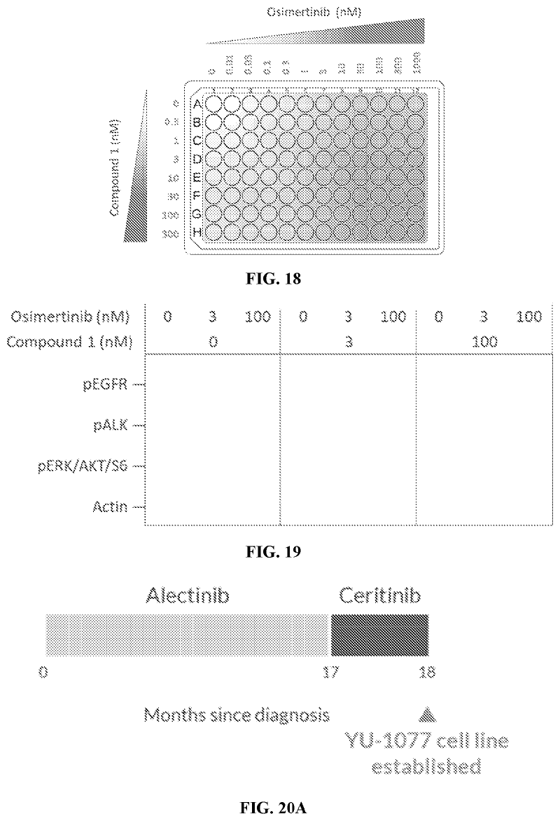

In vitro viability study: MR240 is a cell line derived from a patient with non-small cell lung cancer harboring EGFR ex19del/T790M and STRN-ALK fusion. See Chen J, et al Ann Oncol. 2022 April; 33 (4): 434-444, which is incorporated herein by referenced in its entirety. MR240 cells plated in a 96-well plate (12 columns×8 rows) are treated with increasing concentrations of osimertinib column-wise and Compound 1 row-wise in a semi-3-fold dilution series. The top concentration is 1 μM for osimertinib and 300 nM for Compound 1, with the lowest concentration being vehicle control (0 nM) for both axes. See FIG. 18 for the plate layout. Cells are treated for 3-5 days, after which viability is assessed using the CellTiter-Glo reagent. Effects of the combination treatment are evaluated using the Chou-Talalay index or another equivalent methodology. See Chou, T. C. Cancer Res. 2010 Jan. 15; 70(2):440-6, which is incorporated herein by reference in its entirety.

In vitro signaling pathway study: MR240 cells are treated with 0, 3, or 100 nM osimertinib in the presence of 0, 3, or 100 nM Compound 1 (for a total of 9 conditions, see FIG. 19) for 6 hours. Effects of drug treatment on cell signaling pathways are measured by western blot using antibodies specific to the activated phosphorylated forms of EGFR, ALK, AKT, ERK, and S6, as well as using antibodies specific to their total proteins. Actin is used as a loading control.

Tolerability/pharmacokinetics/pharmacodynamics (tolerability/PK/PD) study: MR240 is also a patient-derived xenograft (PDX) of non-small cell lung cancer harboring EGFR ex19del/T790M and STRN-ALK fusion. See Chen, J. et al Ann Oncol. 2022 April; 33(4):434-444. Balb/c nude mice are implanted subcutaneously in the flank with MR240 PDX. Mice are randomized into six groups based on tumor volume and body weight, and treatment is initiated. Mice are treated with osimertinib (0, 5, or 25 mg/kg QD) in the presence of Compound 1 (0 or 1.5 mg/kg BID), for a total of 6 conditions (see Table 3). Treatment is performed for 11 days, with treatments skipped on weekends (5 on/2 off/4 on). Tumor volume, body weight, and clinical signs are observed. Plasma samples at 1 hour after the first dose are collected to serve as a baseline for determining drug accumulation over time. Plasma samples at 1, 7, and 24 hours after the last dose are collected to determine the exposure of each compound. Tumor samples at 1 and 24 hours after the last dose are collected to measure signaling pathway activity using EGFR, ALK, ERK, AKT, and S6 (phospho or total) antibodies, with actin or GAPDH as a loading control.

TABLE 3
Design of the tolerability/PK/PD study

| | | Osimertinib | Compound 1 | | | |
|---|---|---|---|---|---|---|
| | | (QD, 5 | (BID, 5 | | | |
| Group | N= | ml/kg) | ml/kg) | Regimen | Observations | Sample collection |
| 1 | 5 | Vehicle | Vehicle | PO × | Tumor | PD analysis |
| 2 | 5 | Vehicle | 1.5 mg/kg | 5 days ON, | volume | Frozen and FFPE samples |
| 3 | 5 | 5 mg/kg | Vehicle | 2 days | Body | at 1 h (n = 2) and 24 h (n = 3) |
| 4 | 5 | 25 mg/kg | Vehicle | OFF, | weight | after morning dose on d 12 |
| 5 | 5 | 5 mg/kg | 1.5 mg/kg | 5 days ON | Clinical | PK analysis |
| 6 | 5 | 25 mg/kg | 1.5 mg/kg | | signs | +Plasma at 1 h (n = 5) after |
| | | | | | | morning |
| | | | | | | +Plasma at 1 h (n = 5), 12 h |
| | | | | | | (n = 3), and 24 h (n = 3) after |
| | | | | | | morning dose on d 12 |

Efficacy study: Balb/c nude mice are implanted subcutaneously in the flank with MR240 PDX. Mice are randomized into four groups based on tumor volume and body weight, and treatment is initiated. Mice are treated with osimertinib (0 or 25 mg/kg QD) in the presence of Compound 1 (0 or 1.5 mg/kg BID), for a total of 4 conditions (see Table 4). Treatment is performed for 28-35 days, with treatments skipped on weekends (5 on/2 off). Tumor volume, body weight, and clinical signs are observed. Plasma samples at 1, 7, and 24 hours after the last dose are collected to determine the exposure of each compound.

TABLE 4
Design of the efficacy study

| | | Osimertinib | Compound 1 | | | Sample |
|---|---|---|---|---|---|---|
| Group | N= | (QD, 5 ml/kg) | (BID, 5 ml/kg) | Regimen | Observations | collection |
| 1 | 5 | Vehicle | Vehicle | PO × 28-35 | Tumor | PK |
| 2 | 5 | Vehicle | 1.5 mg/kg | days | volume | analysis |
| 3 | 5 | 25 mg/kg | Vehicle | (5 days | Body | Plasma |
| 4 | 5 | 25 mg/kg | 1.5 mg/kg | ON, 2 | weight | from all |
| | | | | days OFF) | Clinical | mice at |
| | | | | | signs | 1 h, 7 h, |
| | | | | | | and 24 h |
| | | | | | | after final |
| | | | | | | dose |

### Example 18: Preclinical In Vitro and Intracranially Activity of Compound 1 Against YU-1077 Cell Line

Cell line description: YU-1077 is a patient-derived cell line (PDC). YU-1077 was developed from pleural effusion of a patient with NSCLC after progressive disease on alectinib and while on subsequent ceritinib treatment for 1 month. Sanger sequencing confirmed EML4-ALK fusion breakpoint variant 3 (v3) and the G1202R mutation, which is known to confer resistance to alectinib and ceritinib. YU-1077 characterization is shown in FIG. 20A and FIG. 20B.

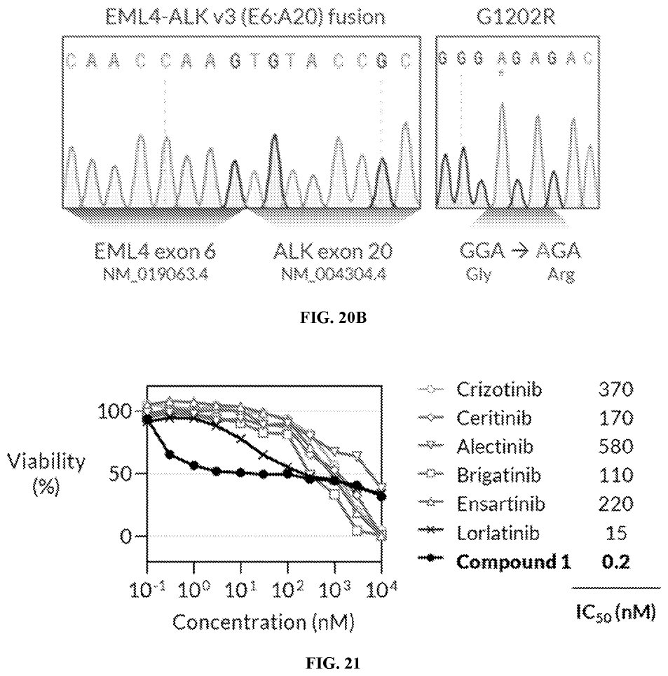

Suppression of cell viability: YU-1077 cells showed limited sensitivity to first- and second-generation ALK TKIs (IC₅₀ range=110-580 nM), consistent with prior relapse on alectinib and with the G1202R resistance mutation. Compound 1 potently inhibited YU-1077 (IC₅₀=0.2 nM), being active against ALK resistance mutations. Compound 1 was 75-fold more potent than lorlatinib (IC₅₀=15 nM) and >500-fold more potent than any other ALK TKIs tested. The results are shown in FIG. 21.

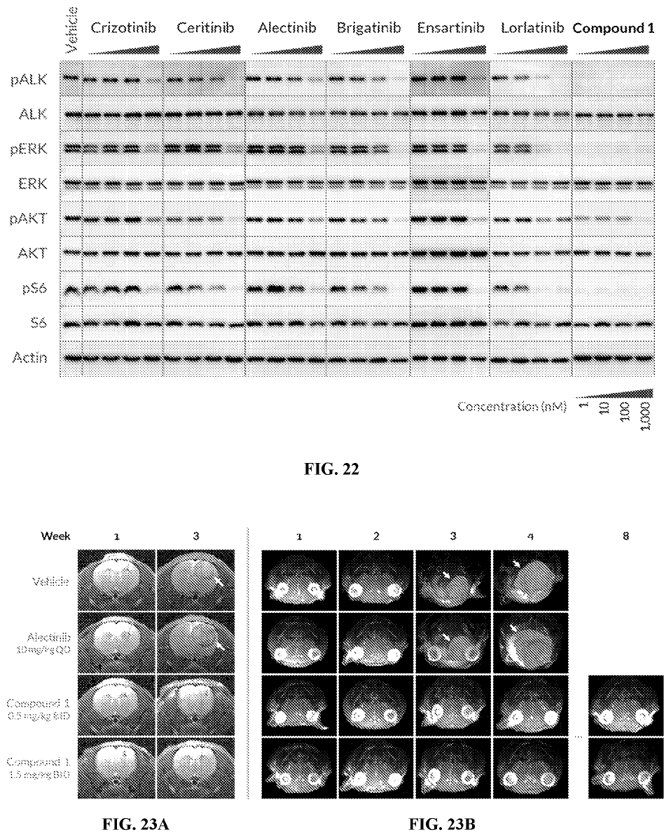

Cell signaling pathway inhibition: Pathway analysis was consistent with inhibition of cell viability and ALK targeting. Among ALK TKIs, only Compound 1 showed complete inhibition of ALK phosphorylation at all concentrations tested, even at the lowest concentration tested (1 nM). Compound 1 also potently inhibited downstream signaling through ERK, AKT, and S6. The results are shown in FIG. 22.

Intracranial model description: Ten thousand YU-1077 cells were stereotactically injected into the brain of Balb/c nude mice, and brain tumor was monitored using magnetic resonance imaging (MRI). This served as a model for drug-resistant lung cancer that has metastasized to the brain, which presented several layers of challenges: (1) The G1202R mutation reduces sensitivity to all approved ALK TKIs. (2) Intracranial tumor may be protected by a blood-brain barrier. (3) YU-1077 is a PDC, which is potentially more heterogeneous, complex, heavily pretreated, and clinically relevant than engineered systems.

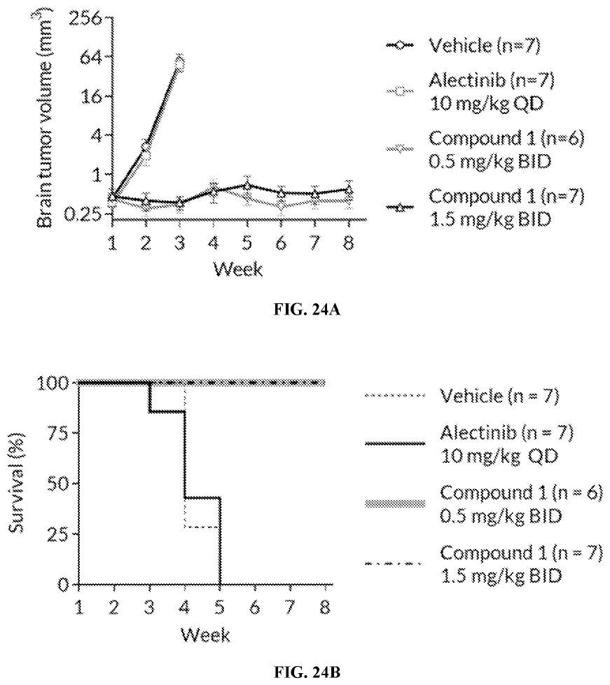

Suppression of intracranial tumor: Vehicle-treated mice showed rapid brain tumor enlargement, from 0.4 to 50 mm³ on average over 2 weeks. Alectinib (10 mg/kg QD) provided no benefit over vehicle on tumor volume or survival, consistent with the G1202R resistance mutation. By contrast, Compound 1 treatment (0.5 or 1.5 mg/kg BID) suppressed brain tumor growth. All mice on Compound 1 treatment survived throughout the study period. The results are shown in FIG. 23A, FIG. 23B, FIG. 24A, and FIG. 24B.

A number of references have been cited, the disclosures of which are incorporated herein by reference in their entirety.

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

What is claimed is:

1. A method of treating a subject with solid tumor, comprising administering to said subject a therapeutically effective amount of Compound 1: 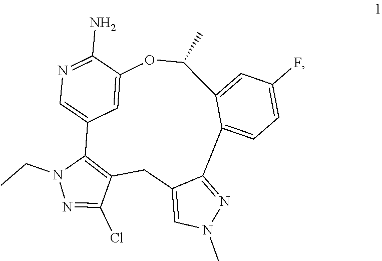 [SMILES: CCn1nc(Cl)c2c1-c1cnc(N)c(c1)O[C@H](C)c1cc(F)ccc1-c1nn(C)cc1C2]
or a stereoisomer, or a mixture of stereoisomers thereof, or a pharmaceutically acceptable salt thereof.

2. The method of claim 1, wherein the solid tumor is advanced solid tumor.

3. The method of claim 2, wherein the advanced solid tumor is relapsed after, refractory to, or resistant to the prior treatment by a tyrosine kinase inhibitor (TKI).

4. The method of any one of claims 1 to 3, wherein the solid tumor is non-small cell lung cancer (NSCLC).

5. The method of any one of claims 1 to 3, wherein the solid tumor is cholangiocarcinoma.

6. The method of any one of claims 1 to 3, wherein the solid tumor is neuroblastoma.

7. The method of any one of claims 1 to 3, wherein the solid tumor is soft-tissue sarcoma.

8. The method of any one of claims 1 to 7, wherein the solid tumor is metastatic.

9. The method of claim 8, wherein the solid tumor is CNS metastatic.

10. The method of any one of claims 1 to 9, wherein the solid tumor is ALK positive.

11. The method of claim 10, wherein the ALK positive solid tumor is characterized by the presence of a mutation in an ALK gene.

12. The method of claim 11, wherein the ALK mutation comprises one or more ALK rearrangement, one or more ALK point mutation, or a combination thereof.

13. The method of claim 11 or claim 12, wherein the ALK mutation comprises one or more ALK fusions.

14. The method of claim 13, wherein the ALK fusion is with one of the fusion partners selected from the group consisting of EML4, TMP1, WDCP, GTF2IRD1, TPM3, TPM4, CLTC, LMNA, PRKAR1A, RANBP2, TFG, FN1, KLC1, VCL, STRN, HIP1, NPM1, DCTN1, SQSTM1, TPR, CRIM1, PTPN3, FBXO36, ATIC, MSN, ALO17, MYH9 and KIF5B.

15. The method of claim 14, wherein the ALK fusion is with NPM1, STRN, or EML4.

16. The method of any one of claims 11 to 15, wherein the ALK mutation comprises G1202R, F1174C, F1174L, I1171N, I1171S, I1171T, L1196M, V1180L, C1156Y, G1202del, G1202K, G1269A, G1269V, F1174S, F1174I, S1206Y, E1210K, T1151M, T1151_L1152insT, D1203N, S1206C, L1152R, L1196Q, L1198P, L1198F, R1275Q, L1152P, C1156T, E1129K, S1206F, L1198H, F1245C, T1151K, I1268V, F1174V, L1198Q, S1206A or F1245V, or a combination thereof.

17. The method of claim 16, wherein the ALK mutation comprises G1202R.

18. The method of claim 16, wherein the ALK mutation comprises F1174S or F1174L.

19. The method of claim 16, wherein the ALK mutation comprises R1275Q.

20. The method of claim 16, wherein the ALK mutation comprises T1151M.

21. The method of any one of claims 11 to 16, wherein the ALK mutation comprises one or more compound mutations.

22. The method of claim 21, wherein the compound mutation is G1202R/F1174L.

23. The method of claim 21, wherein the compound mutation is G1202R/T1151M, G1202R/L1196M, G1202R/G1269A, G1202R/L1198F, or G1202R/F1174S.

24. The method of claim 21, wherein the compound mutation is C1156Y/L1256F, C1156Y/S1206F, C1156Y/F1174V, or C1156Y/F1174I.

25. The method of claim 21, wherein the compound mutation is L1196M/L1198H, L1196M/I1179V, or L1196M/L1256F.

26. The method of claim 10, wherein the ALK positive solid tumor is characterized by the presence of a partially deleted ALK protein.

27. The method of any one of claims 1 to 26, wherein the subject is naïve to tyrosine kinase inhibitor (TKI) therapy.

28. The method of any one of claims 1 to 26, wherein the subject has been treated with one prior TKI therapy.

29. The method of any one of claims 1 to 26, wherein the subject has been treated with at least one prior TKI therapy.

30. The method of any one of claims 1 to 26, wherein the subject has been treated with at least two prior TKI therapies.

31. The method of any one of claims 27 to 30, wherein the TKI is ALK TKI.

32. The method of claim 31, wherein the ALK TKI is crizotinib, ceritinib, alectinib, brigatinib, or lorlatinib.

33. The method of any one of claims 1 to 32, wherein the subject has been treated with one or more prior systemic anticancer therapies.

34. The method of any one of claims 1 to 33, wherein the subject has not been treated with prior chemotherapy.

35. The method of any one of claims 1 to 33, wherein the subject has been treated with prior chemotherapy.

36. The method of claim 35, wherein the subject has been treated with up to two lines of prior chemotherapy.

37. The method of any one of claims 1 to 36, wherein the subject has not been treated with prior immunotherapy.

38. The method of any one of claims 1 to 36, wherein the subject has been treated with prior immunotherapy.

39. The method of claim 38, wherein the subject has been treated with up to two lines of prior immunotherapy.

40. The method of claim 1, wherein the solid tumor is advanced or metastatic ALK positive NSCLC, and the subject has been treated with at least one prior ALK TKI therapy.

41. The method of claim 40, wherein at least one prior ALK TKI therapy is ceritinib, crizotinib, alectinib, brigatinib, or lorlatinib.

42. The method of claim 1, wherein the solid tumor is advanced or metastatic ALK positive NSCLC, and the subject has been treated with one prior ALK TKI therapy.

43. The method of claim 42, wherein the prior ALK TKI therapy is ceritinib, crizotinib, alectinib, brigatinib, or lorlatinib.

44. The method of claim 1, wherein the solid tumor is advanced or metastatic ALK positive NSCLC, and the subject has been treated with two or three prior ALK TKI therapies.

45. The method of claim 44, wherein the two or three prior ALK TKI therapies are crizotinib, ceritinib, alectinib, brigatinib, and lorlatinib.

46. The method of claim 44 or 45, wherein the second or third prior ALK TKI therapy is lorlatinib.

47. The method of any one of claims 40 to 46, wherein the subject has been treated with ≤2 prior lines of chemotherapy and/or immunotherapy.

48. The method of any one of claims 40 to 46, wherein the subject has been treated with more than two prior lines of chemotherapy and/or immunotherapy.

49. The method of claim 1, wherein the solid tumor is an advanced or metastatic ALK positive solid tumor, and the subject has been treated with one or more prior systemic anticancer therapies.

50. The method of claim 1, wherein the solid tumor is advanced or metastatic ALK positive NSCLC, and the subject has progressed on a prior therapy.

51. The method of any one of claims 1 to 50, wherein Compound 1 is administered to the patient for one or more days.

52. The method of any one of claims 1 to 51, wherein Compound 1 is administered to the patient for at least one treatment cycle.

53. The method of claim 52, wherein one treatment cycle is at least 7 days.

54. The method of claim 52, wherein one treatment cycle is at least 14 days.

55. The method of claim 52, wherein one treatment cycle is at least 21 days.

56. The method of any one of claims 1 to 55, wherein the patient does not experience a Grade 4 adverse event (e.g., TRAE) after the administration of Compound 1.

57. The method of any one of claims 1 to 56, wherein the patient does not experience a Grade 3 adverse event (e.g., TRAE) after the administration of Compound 1.

58. The method of any one of claims 1 to 57, wherein the patient does not experience a Grade 2 adverse event (e.g., TRAE) after the administration of Compound 1.

59. The method of any one of claims 1 to 58, wherein the patient does not experience a Grade 1 adverse event (e.g., TRAE) after the administration of Compound 1.

60. The method of any one of claims 1 to 58, wherein the patient experiences at most a Grade 1 adverse event (e.g., TRAE) after the administration of Compound 1.

61. The method of any one of claims 1 to 60, wherein the patient does not experience a CNS adverse event after the administration of Compound 1.

62. The method of claim 61, wherein the CNS adverse event is one or more selected from the group consisting of dizziness, ataxia, gait disturbance, paraesthesia, weight gain, hyperphagia, paresthesias, abnormal movement, cognitive changes, speech effects (e.g., dysarthria, slow speech, or speech disorder), mood disorder (e.g., irritability, anxiety, depression, affect lability, personality change, mood swings, affective disorder, aggression, agitation, mood altered, depressed mood, euphoric mood, or mania), and cognitive disorder (e.g., memory impairment, cognitive disorder, amnesia, confusion, disturbance in attention, delirium, mental impairment, attention deficit/hyperactivity disorder, dementia, sleep disturbance, or reading disorder).

63. The method of any one of claims 1 to 62, wherein the patient does not experience an adverse event of weight gain and/or glucose metabolism disorders.

64. The method of any one of claims 1 to 63, wherein the patient is a patient population.

65. The method of claim 64, wherein the patient population experiences no Grade 3 or Grade 4 TRAE after administration of Compound 1.

66. The method of any one of claims 1 to 65, wherein the patient has a complete response after one or more cycles of treatment.

67. The method of any one of claims 1 to 65, wherein the patient has a partial response after one or more cycles of treatment.

68. The method of any one of claims 1 to 67, wherein the patient has reached stable disease after one or more cycles of treatment.

69. The method of any one of claims 1 to 68, wherein the patient has brain metastases.

70. The method of any one of claims 1 to 69, wherein the patient has brain metastases and experiences no intracranial progression after at least one treatment cycle.

71. The method of any one of claims 1 to 70, wherein the patient has at least about 5% to about 100% reduction of ALK allele variant in circulating tumor DNA after at least one treatment cycle.

72. The method of any one of claims 1 to 71, wherein the patient has at least about 30% reduction of ALK allele variant in circulating tumor DNA after at least one treatment cycle.

73. The method of any one of claims 1 to 72, wherein the patient has at least about 50% reduction of ALK allele variant in circulating tumor DNA after at least one treatment cycle.

74. The method of any one of claims 1 to 73, wherein the patient has at least about 70% reduction of ALK allele variant in circulating tumor DNA after at least one treatment cycle.

75. The method of any one of claims 1 to 74, wherein the patient has at least about 100% reduction of ALK allele variant in circulating tumor DNA after at least one treatment cycle.

76. The method of any one of claims 1 to 75, wherein the patient has undetectable ALK allele variant in circulating tumor DNA after at least one treatment cycle.

77. The method of claim 76, wherein the ALK allele variant is G1202R.

78. The method of any one of claims 1 to 49, wherein the compound is administered at an amount of from about 5 mg to about 400 mg (by weight of Compound 1 free base) once daily.

79. The method of claim 78, wherein the compound is administered at an amount of from about 15 mg to about 200 mg (by weight of free base Compound 1) once daily.

80. The method of claim 79, wherein the compound is administered at an amount of from about 25 mg to about 200 mg (by weight of free base Compound 1) once daily.

81. The method of claim 80, wherein the compound is administered at an amount of from about 15 mg to about 150 mg (by weight of free base Compound 1) once daily.

82. The method of claim 81, wherein the compound is administered at an amount of from about 15 mg to about 100 mg (by weight of free base Compound 1) once daily.

83. The method of claim 78, wherein the compound is administered at an amount of about 10 mg, about 15 mg, about 25 mg, about 50 mg, about 100 mg, about 150 mg, about 200 mg, or about 250 mg (by weight of free base Compound 1) once daily.

84. The method of any one of claims 1 to 49, wherein the compound is administered at an amount of from about 5 mg to about 400 mg (by weight of Compound 1 free base) twice daily (BID).

85. The method of claim 84, wherein the compound is administered at an amount of from about 15 mg to about 200 mg (by weight of free base Compound 1) twice daily (BID).

86. The method of claim 85, wherein the compound is administered at an amount of from about 25 mg to about 200 mg (by weight of free base Compound 1) twice daily (BID).

87. The method of claim 86, wherein the compound is administered at an amount of from about 15 mg to about 150 mg (by weight of free base Compound 1) twice daily (BID).

88. The method of claim 87, wherein the compound is administered at an amount of from about 15 mg to about 100 mg (by weight of free base Compound 1) twice daily (BID).

89. The method of claim 84, wherein the compound is administered at an amount of about 10 mg, about 15 mg, about 25 mg, about 50 mg, about 100 mg, about 150 mg, about 200 mg, or about 250 mg (by weight of free base Compound 1) twice daily (BID).

90. The method of any one of claims 1 to 89, wherein the compound is administered orally.

91. The method of claim 90, wherein the compound is administered in the form of one or more tablets.

92. The method of claim 91, wherein the tablet has a unit dose strength of about 5 mg, about 50 mg, about 75 mg, about 100 mg, about 125 mg, or about 150 mg by weight of free base Compound 1.

93. The method of any one of claims 90 to 92, wherein the compound is administered to a patient with an empty stomach.

94. The method of any one of claims 90 to 92, wherein the compound is administered to a patient with a full stomach.

95. The method of any one of claims 1 to 94, wherein the subject is not taking any one of strong inducers of CYP3A4, strong inhibitors of CYP3A4, sensitive substrates of CYP3A4 and/or CYP2C8, substrates of P-gp/multidrug resistance protein (MDR1), substrates of BCRP/breast cancer resistance protein (ABCG2), substrates of OATP1B1, substrates of OATP1B3, substrates of MATE1, or gastric acid reducing agents.

96. The method of any one of claims 1 to 94, wherein the subject is taking any one of strong inducers of CYP3A4, strong inhibitors of CYP3A4, sensitive substrates of CYP3A4 and/or CYP2C8, substrates of P-gp/multidrug resistance protein (MDR1), substrates of BCRP/breast cancer resistance protein (ABCG2), substrates of OATP1B1, substrates of OATP1B3, substrates of MATE1, or gastric acid reducing agents.

97. The method of any one of claims 1 to 94, wherein the subject is taking any one of strong inducers of CYP3A4 or strong inhibitors of CYP3A4.

98. The method of any one of claims 1 to 94, wherein the subject is not taking any one of strong inducers of CYP3A4 or strong inhibitors of CYP3A4.

99. The method of any one of claims 1 to 94, wherein the subject is not taking any one of strong inducers of CYP3A4.

100. The method of any one of claims 1 to 99, wherein the subject experiences improvement in one or more symptoms selected from the group consisting of cognitive impairment, mood disorders, sleep disturbances, dizziness, ataxia, and weight gain, after the administration of the compound.

101. The method of any one of claims 1 to 99, wherein the subject does not experience one or more symptoms selected from the group consisting of cognitive impairment, mood disorders, sleep disturbances, dizziness, ataxia, and weight gain, after the administration of the compound.

102. The method of any one of claims 1 to 101, wherein the subject experiences reduced levels of one or more of pALK, pERK, pAKT, and phospho-S6, after the administration of the compound.

103. The method of any one of claims 1 to 102, wherein the subject experiences increased levels of cleaved PARP, after the administration of the compound.

104. The method of any one of claims 1 to 103, wherein the subject experiences reduced activity of MAP kinase pathway, PI3K/AKT pathway, or JAK/STAT pathway, or any combination thereof in tumor, after the administration of the compound.

105. The method of any one of claims 1 to 104, wherein the subject experiences reduced activity of MAP kinase pathway, PI3K/AKT pathway, or JAK/STAT pathway, or any combination thereof in solid tumor, after the administration of the compound.

106. The method of any one of claims 1 to 105, wherein the subject experiences increased expression level of one or more marker of apoptosis in tumor, after the administration of the compound.

107. The method of any one of claims 1 to 106, wherein the subject experiences decreased level of one or more marker of proliferation in tumor, after the administration of the compound.

108. The method of any one of claims 1 to 106, wherein the subject experiences increased expression level of one or more marker of apoptosis in solid tumor, after the administration of the compound.

109. The method of claim 1, wherein the solid tumor is leukocyte receptor tyrosine kinase (LTK) positive.

110. The method of claim 109, wherein the solid tumor is LTK positive lung cancer.

111. The method of claim 110, wherein the solid tumor is LTK positive NSCLC.

112. The method of any one of claims 1 to 111, wherein the compound is Compound 1 free base.

113. The method of any one of claims 1 to 112, wherein the compound is a solid form of Compound 1.

114. The method of claim 113, wherein the solid form is characterized by an XRPD pattern comprising peaks at approximately 12.4, 18.9, and 21.1° 2θ (±0.2°).

115. The method of any one of claims 1 to 114, wherein the subject is administered a pharmaceutical composition comprising the therapeutically effective amount of Compound 1, wherein the pharmaceutical composition further comprises a diluent, a disintegrant, a glidant, a binder, and a lubricant.

116. The method of claim 115, wherein the diluent is microcrystalline cellulose, the disintegrant is croscarmellose sodium, the glidant is colloidal silica dioxide, the binder is hydroxypropyl cellulose (HPC), and the lubricant is magnesium stearate.
